(19) United States
(12) Patent Application Publication
(10) Pub. No.: US 2013/0190399 A1
(43) Pub. Date: Jul. 25, 2013

(54) Synthesis and use of omega-3 and omega 6 very long chain polyunsaturated fatty acids (VLC-PUFA)

(71) Applicants: Krishna Raman, Wilmington, DE (US); Vinod Tarwade, Rockville, MD (US)

(72) Inventors: Krishna Raman, Wilmington, DE (US); Vinod Tarwade, Rockville, MD (US)

(73) Assignee: MARTEK BIOSCIENCES CORPORATION, Columbia, MD (US)

(21) Appl. No.: 13/460,536

(22) Filed: Apr. 30, 2012

Related U.S. Application Data
(63) Continuation-in-part of application No. PCT/US10/54901, filed on Oct. 31, 2010.
(60) Provisional application No. 61/481,598, filed on May 2, 2011.
(60) Provisional application No. 61/256,969, filed on Oct. 31, 2009.

(51) Int. Cl. C07C 67/343; A61K 31/202; C07C 51/377; C07D 309/12; C07C 51/09

(52) U.S. Cl. CPC C07C 67/343; C07D 309/12; C07C 51/09; C07C 51/377; A61K 31/202
USPC 514/560; 549/416; 554/153; 554/132; 554/224

(57) ABSTRACT

The invention provides methods of synthesizing omega-3 and omega-6 very long chain polyunsaturated fatty acids (VLC-PUFAs, C28-C42:4, 5 and 6), analogs and derivatives thereof, pharmaceutical compositions containing these isolated VLC-PUFA compounds and therapeutic uses therefor.

### CROSS REFERENCE TO RELATED APPLICATION

This application is a continuation-in-part of International Application No. PCT/US10/54901 filed on Oct. 31, 2010, which claims the benefit of priority under 35 U.S.C. §119(e) to U.S. Provisional Patent Application Ser. No. 61/256,969 filed Oct. 31, 2009. Each of these priority documents are incorporated herein by reference in their entirety.

### TECHNICAL FIELD

The invention relates to the synthesis of omega-3 and omega-6 very long chain polyunsaturated fatty acids (VLC-PUFAs, C28-C42:4, 5 and 6) and analogs and derivatives thereof, pharmaceutical compositions containing the same and therapeutic uses therefor.

### BACKGROUND OF INVENTION

Polyunsaturated fatty acids (“PUFAs”) are categorized according to the number and position of double bonds in the fatty acids according to an accepted nomenclature that is well known to those of ordinary skill in the art. There are two series or families of PUFAs, depending on the position of the double bond closest to the methyl end of the fatty acid: the n-3 series contains a double bond at the third carbon, while the n-6 series has no double bond until the sixth carbon. Thus, arachidonic acid (AA or ARA) has a chain length of 20 carbons and 4 double bonds beginning at the sixth carbon, and is referred to as “20:4 n-6.” Similarly, docosahexaenoic acid (DHA) has a chain length of 22 carbons with 6 double bonds beginning with the third carbon from the methyl end and is thus designated “22:6 n-3.” Another important LC-PUFA is eicosapentaenoic acid (EPA) which is designated (20:5 n-3). The terms “n-3” and “omega-3” are used interchangeably as are the terms “n-6” and “omega-6.” Other important PUFAs are the C-18 fatty acids, for example, linoleic (18:2 n-6), gamma-linolenic acid (18:3 n-6) in the n-6 pathway, and alpha-linolenic (18:3 n-3) and stearidonic (18:4 n-3) in the n-3 pathway. These shorter PUFAs serve as precursors to the longer PUFAs in biosynthetic pathways that share elongation and desaturation steps and are well understood to those of skill in the art.

Omega-3 fatty acids include synthetic or naturally occurring omega-3 fatty acids, such as those found in fish oil, e.g., marine mammal (e.g., seal) fat, cod liver oil, walnuts and walnut oil, wheat germ oil, rapeseed oil, soybean lecithin, soybeans, tofu, common beans, butternuts, seaweed and flax seed oil. An omega-3 fatty acid may also be derived from genetically engineered sources such as transgenic plants.

Since the detection of DHA in the retina nearly forty years ago, extensive research efforts have documented its vital role in retinal physiology. In particular, multiple studies have shown that a baby milk formula which was deficient in DHA and its n-3 precursors delayed development of vision in neonates. Other studies reported that low tissue levels of DHA were associated with Retinitis Pigmentosa, Usher's syndrome Type I and the severity of Stargardt disease-3. These findings have lead to DHA supplementation of commercial baby milk formulas and experimental treatments of retinal pathologies using DHA dietary supplementation.

In addition to DHA, it has also been discovered that the retina contains a unique group of polyunsaturated C28-C36 fatty acids. In fact, omega-3 and omega-6 very long chain polyunsaturated fatty acids (“VLC PUFAs”; C28-42:4, 5 and 6) have been found in mammalian retina, brain and sperm as part of phospholipids, ceramides and sphingomyelins. Preliminary reports suggest that Stargardt disease-3 is the result of deficiency of retinal polyunsaturated C28-C36 fatty acids and retinal health requires these fatty acids, in addition to DHA.

VLC-PUFAs are at most a minor component of the human diet and, in mammalian systems, are believed to be formed by elongation of fatty acids including both omega-3 and omega-6 PUFAs. Synthesis of fatty acids up to C16 is catalyzed by the cytoplasmic fatty acid synthase complex and these fatty acids can be further elongated by the four consecutive enzymatic reactions shown in FIG. 1. The first of these, condensation of a fatty acyl-CoA with malonyl-CoA, is catalyzed by one of seven known human elongases, each of which catalyzes substrate specific elongation of fatty acids of different lengths and degrees of saturation.

ELOVL4 is the elongase known to synthesize fatty acids with carbon chain lengths of 28 or longer. This enzyme has been shown to synthesize polyunsaturated C28-C36 fatty acids in the mouse retinal, and saturated and monounsaturated fatty acids in the mouse epidermis. The condensation of a fatty acyl-CoA with malonyl-CoA is the rate-limiting step of long chain fatty acid biosynthesis. ELOVL4 catalyzes this rate-limiting condensation reaction, and the heterozygous ELOVL4 mutation in Stargardt-3 mice results in reduced levels of retinal C32-C36 PUFAs. In addition, four mouse lines with various heterozygous ELOVL4 mutations all show a selective reduction of epidermal acylceramides containing C28-C36 PUFAs. Methods of using these enzyme reactions to produce C28-C40 PUFAs in a recombinantly engineered host organism have been disclosed U.S. Patent Publication No. 2009/0203787 A1.

Unfortunately, due to the limited enzymatic production rate and the limited amount of VLC-PUFAs found in the few known biological sources, study of these compounds and their therapeutic usefulness has been very limited. Therefore, there is a need for reliable and efficient chemical (i.e. non-biological) methods of producing VLC-PUFAs, analogs, and derivatives, as well as compositions containing these compounds for therapeutic administration.

### SUMMARY OF INVENTION

Conventional sources of VLC-PUFAs, such as retina, brain and sperm, have only extremely small amounts of these long chain fatty acids. The present inventors have realized that the provision of compositions enriched in VLC-PUFAs would be of great benefit to human nutrition and health and would provide an alternative to the natural production or accumulation of these PUFAs by an organism. The present inventors have therefore discovered alternative ways to produce these VLC-PUFAs for the preparation of compositions enriched in VLC-PUFAs that have significantly enhanced levels over naturally occurring tissue sources of VLC-PUFAs. Thus, the present invention is drawn to chemical methods of producing omega-3 and omega-6 very long chain polyunsaturated fatty acids (“VLC-PUFAs”; C28-42:4, 5 and 6), derivatives and compositions containing these VLC-PUFAs that are particularly useful in the administration of VLC PUFAs for therapeutic purposes.

One aspect of the invention provides a method of coupling a long chain hydrocarbon to an extender hydrocarbon, in at least one step, to form a very long chain hydrocarbon having at least 28 carbon atoms. In this method, a long chain hydrocarbon that has a nucleophilic displacement group on one end is contacted with an extending reagent, in at least one step, to form a very long chain hydrocarbon having at last 28 carbon atoms.

The long chain hydrocarbon starting material may be a C20 PUFA, such as ARA or EPA, or a C22 PUFA, such as DPA or DHA. In similar embodiments, DHASCO-T and DHASCO-S oils (Martek Biosciences Corporation, Columbia, Md.) may be used as a source of long chain hydrocarbon starting materials.

The extending reagent comprises an extender hydrocarbon chain with a nucleophillic attacking group on one end and a protecting functional group on the opposite end of the hydrocarbon chain. In particular embodiments, the extender hydrocarbon may be one of a C2, C4, C6, or C8 hydrocarbon. In some embodiments, the contacting of the long chain hydrocarbon with the extender hydrocarbon is conducted in the presence of an activating catalyst.

In a particular embodiment of this aspect of the invention, the protecting functional group is one of an ester or an ether.

In another embodiment of this aspect of the invention, the nucleophillic displacement group is a halogen.

In another embodiment of this aspect of the invention, the extending reagent is one of a Grignard extender reagent and a zinc extender reagent.

In another aspect of the invention, the protecting functional group is an ether, and the ether formed in the coupling reaction is converted to an alcohol. In another related aspect of the invention, the alcohol is converted to a free fatty acid. In yet another related aspect of the invention, the free fatty acid is converted to an ester. In a further related aspect, the free fatty acid is converted to an ethyl ester.

In another aspect of the invention, the protecting functional group is an ester, and the ester formed in the coupling reaction is converted to a free fatty acid. In a related aspect of the invention, the free fatty acid is converted to an ethyl ester.

Another aspect of the invention is a chemically-synthesized VLC-PUFA. In this aspect, the VLC-PUFA has a chain length of 28, 30, 32, 34, 36, 38, 40 or 42 carbons. In particular embodiments, the VLC-PUFA may be any one of the (C28-C42) n-3 fatty acids, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 fatty acids, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these chemically-synthesized VLC-PUFAs. In certain embodiments, this composition contains greater than about 80% of a specific VLC-PUFA. In certain embodiments, this composition contains greater than about 90% of a specific VLC-PUFA.

Another aspect of the invention is a VLC-PUFA alcohol. In this aspect, the VLC-PUFA alcohol has a chain length of 28, 30, 32, 34, 36, 38, 40 or 42 carbons. In particular embodiments, the VLC-PUFA alcohol may be any one of the (C28-C42) n-3 fatty acids, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 fatty acids, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these VLC-PUFA alcohols. In certain embodiments, this composition contains greater than about 80% of a specific VLC-PUFA alcohol. In certain embodiments, this composition contains greater than about 90% of a specific VLC-PUFA alcohol.

Another aspect of the invention is a chemically-synthesized VLC-PUFA alcohol. In this aspect, the VLC-PUFA alcohol has a chain length of 28, 30, 32, 34, 36, 38, 40 or 42 carbons. In particular embodiments, the VLC-PUFA alcohol may be any one of the (C28-C42) n-3 fatty alcohols, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 fatty alcohols, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these chemically-synthesized VLC-PUFA alcohols. In certain embodiments, this composition contains greater than about 80% of a specific VLC-PUFA alcohol. In certain embodiments, this composition contains greater than about 90% of a specific VLC-PUFA alcohol.

Another aspect of the invention is a VLC-PUFA free fatty acid. In this aspect, the VLC-PUFA free fatty acid has a chain length of 28, 30, 32, 34, 36, 38, 40 or 42 carbons. In particular embodiments, the VLC-PUFA free fatty acid may be any one of the (C28-C42) n-3 free fatty acids, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 free fatty acids, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these VLC-PUFA free fatty acid. In certain embodiments, this composition contains greater than about 80% of a specific VLC-PUFA free fatty acid. In certain embodiments, this composition contains greater than about 90% of a specific VLC-PUFA free fatty acid.

Another aspect of the invention is a chemically-synthesized VLC-PUFA free fatty acid. In this aspect, the VLC-PUFA free fatty acid has a chain length of 28, 30, 32, 34, 36, 38, 40 or 42 carbons. In particular embodiments, the VLC-PUFA free fatty acid may be any one of the (C28-C42) n-3 fatty acids, including but not limited to, 28:4n3, 28:5n3, 28:6n3, 30:4n3, 30:5n3, 30:6n3, 32:4n3, 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 free fatty acids, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these chemically-synthesized VLC-PUFA free fatty acid. In certain embodiments, this composition contains greater than about 80% of a specific VLC-PUFA free fatty acid. In certain embodiments, this composition contains greater than about 90% of a specific VLC-PUFA free fatty acid.

Another aspect of the invention is a VLC-PUFA ethyl ester. In this aspect, the VLC-PUFA ethyl ester has a chain length of 28, 30, 32, 34, 36, 38, 40 or 42 carbons. In particular embodiments, the VLC-PUFA ethyl ester may be any one of the (C28-C42) n-3 ethyl esters, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 ethyl esters, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these VLC-PUFA ethyl ester. In certain embodiments, this composition contains greater than about 80% of a specific VLC-PUFA ethyl ester. In certain embodiments, this composition contains greater than about 90% of a specific VLC-PUFA ethyl ester.

Another aspect of the invention is a chemically-synthesized VLC-PUFA ethyl ester. In this aspect, the VLC-PUFA ethyl ester has a chain length of 28, 30, 32, 34, 36, 38, 40 or 42 carbons. In particular embodiments, the VLC-PUFA ethyl ester may be any one of the (C28-C42) n-3 ethyl esters, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 ethyl esters, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these chemically-synthesized VLC-PUFA ethyl ester. In certain embodiments, this composition contains greater than about 80% of a specific VLC-PUFA ethyl ester. In certain embodiments, this composition contains greater than about 90% of a specific VLC-PUFA ethyl ester.

Another aspect of the invention is a VLC-PUFA trigylceride. In this aspect, the VLC-PUFA triglyceride has a chain length of 28, 30, 32, 34, 36, 38, 40 or 42 carbons. In particular embodiments, the VLC-PUFA triglyceride may be any one of the (C28-C42) n-3 triglycerides, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 triglycerides, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these VLC-PUFA triglyceride. In certain embodiments, this composition contains greater than about 80% of a specific VLC-PUFA triglyceride. In certain embodiments, this composition contains greater than about 90% of a specific VLC-PUFA triglyceride.

Another aspect of the invention is a chemically-synthesized VLC-PUFA triglyceride. In this aspect, the VLC-PUFA triglyceride has a chain length of 28, 30, 32, 34, 36, 38, 40 or 42 carbons. In particular embodiments, the VLC-PUFA triglycerides may be any one of the (C28-C42) n-3 triglycerides, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 triglycerides, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these chemically-synthesized VLC-PUFA triglycerides. In certain embodiments, this composition contains greater than about 80% of a specific VLC-PUFA triglyceride. In certain embodiments, this composition contains greater than about 90% of a specific VLC-PUFA triglyceride.

Another aspect of the invention is a VLC-PUFA phospholipid. In this aspect, the VLC-PUFA phospholipid has a chain length of 28, 30, 32, 34, 36, 38, 40 or 42 carbons. In particular embodiments, the VLC-PUFA phospholipid may be any one of the (C28-C42) n-3 phospholipids, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 phospholipids, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these VLC-PUFA phospholipid. In certain embodiments, this composition contains greater than about 80% of a specific VLC-PUFA phospholipid. In certain embodiments, this composition contains greater than about 90% of a specific VLC-PUFA ethyl ester.

Another aspect of the invention is a chemically-synthesized VLC-PUFA phospholipid. In this aspect, the VLC-PUFA ethyl ester has a chain length of 28, 30, 32, 34, 36, 38, 40 or 42 carbons. In particular embodiments, the VLC-PUFA phospholipids may be any one of the (C28-C42) n-3 phospholipids, including but not limited to, 28:4n3, 28:5n3, 28:6n3, 30:4n3, 30:5n3, 30:6n3, 32:4n3 32:5n3, 32:6n3, 34:4n3, 34:5n3, 34:6n3, 36:4n3, 36:5n3, 36:6n3, 38:4n3, 38:5n3, 38:6n3, 40:4n3, 40:5n3, 40:6n3, 42:4n3, 42:5n3, 42:6n3 and any one of the (C28-C40) n-6 fatty acids, including but not limited to, 28:3n6, 28:4n6, 28:5n6, 28:6n6, 30:4n6, 30:5n6, 30:6n6, 32:4n6, 32:5n6, 32:6n6, 34:4n6, 34:5n6, 34:6n6, 36:4n6, 36:5n6, 36:6n6, 38:4n6, 38:5n6, 38:6n6, 40:4n6, 40:5n6, 40:6n6, 42:4n6, 42:5n6, and 42:6n6. A related aspect is a composition containing at least one of these chemically-synthesized VLC-PUFA phospholipids. In certain embodiments, this composition contains greater than about 80% of a specific VLC-PUFA phospholipid. In certain embodiments, this composition contains greater than about 90% of a specific VLC-PUFA phospholipid.

In certain DHA derived VLC-PUFA, the composition is substantially free of at least one EPA and ARA. For example, some embodiments contain less than about 2%, less than about 1%, less than about 0.5%, less than about 0.1% or less than about 0.01% of at least one of ARA and EPA. In some DHA-derived embodiments there is no detectable EPA. In some DHA-derived embodiments there is no detectable ARA.

In certain embodiments, the VLC-PUFA is provided substantially free of any other VLC-PUFAs. For example, some embodiments contain less than about 2%, less than about 1%, less than about 0.5%, less than about 0.1% or less than about 0.01% of any other VLC-PUFA.

This Summary of the Invention is neither intended nor should it be construed as being representative of the full extent and scope of the present invention. Moreover, references made herein to “the present invention,” or aspects thereof, should be understood to mean certain embodiments of the present invention and should not necessarily be construed as limiting all embodiments to a particular description. The present invention is set forth in various levels of detail in this Summary as well as in the attached drawings and the Description of the Embodiments and no limitation as to the scope of the present invention is intended by either the inclusion or non-inclusion of elements, components, etc. in this Summary. Additional aspects of the present invention will become more readily apparent from the Description of Embodiments, particularly when taken together with the figures.

### BRIEF DESCRIPTION OF DRAWINGS

FIG. 1, shows the biosynthetic pathway leading to elongation of C16 fatty acids.

FIG. 2, shows the MS/MS Spectrum of the DHA derived compound formed in the synthesis scheme described in Example 1.

FIG. 3 shows the IR Spectrum of the DHA derived compound formed in the synthesis scheme described in Example 1.

FIG. 4 shows A) LC-MS data and B) NMR data, for the C28:6 alcohol formed by the coupling reaction of DHA bromide with 6-tetrahydropyranyloxyhexyl magnesium chloride catalyzed by Li₂CuCl₄ described in detail in Example 2.

FIG. 5 shows the NMR data of the C28:6 bromide formed by bromination of the C28:6 described in detail in Example 2.

FIG. 6 shows A) LC-MS data and B) NMR data, for the C34:6 alcohol formed in the coupling reaction of C28:6 bromide with 6-tetrahydropyranyloxyhexyl magnesium chloride catalyzed by Li₂CuCl₄ and described in detail in Example 2.

FIG. 7 shows the LC-MS data for C34:6 acid formed by oxidation of the C34:6 alcohol, as described in Example 2.

FIG. 8 shows A) LC-MS data and B) NMR data, for the C34:6 ethyl ester formed from the esterification of C34:6 acid as described in Example 2.

FIG. 9 shows A) LC-MS data and B) NMR data, for the C34:5 acid formed by the oxidation of C34:5 alcohol, as described in Example 3.

FIG. 10 shows the NMR spectrum for the C34:5 ethyl ester by the esterification of the C34:5 acid as described in Example 3.

FIG. 11 shows the HPLC-MS analysis of 34:6 and deuterated 3-34:5 (D3-C34:6), monitored selectively.

FIG. 12 shows the HPLC-MS analysis of 34:5 and D3-34:5, monitored selectively.

FIG. 13 shows the amount of 34:6 and 34:5 present in homogenates from rat livers taken from animals treated as described in Example 4. (1-2=control group, 3-4-5-6=rats treated with 34:5, 7-8-9-10=rats treated with 34:6). The height of the bars represents the amount of analyte present in each sample (expressed as the ratio of the area of the chromatographic peak of a specific VLC-PUFA and its corresponding deuterated reference standard).

FIG. 14 shows the amount of 34:6 and 34:5 present in homogenates from rat testis taken from animals treated as described in Example 4. The VLC-PUFA is detected only in the experimental group that received the treatment.

FIG. 15 shows the amount of 34:6 and 34:5 present in homogenates from rat eyes taken from animals treated as described in Example 4.

FIG. 16 shows the determination of 34:6 and 34:5 levels in heart liver homogenate (as seen for liver and testis, accumulation is evident only in treated animals).

FIG. 17 shows the amount of 34:6 and 34:5 in samples obtained from blood withdrawn from test animals at different days of treatment. The panel on the left shows data obtained from each of the four animals of the group treatment #3 (those who received 34:6 esters) at day 0, 1, 5 and 10 of the treatment. The panel on the right shown data obtained from each of the four animal of experimental group #2 (those receiving 34:5 esters).

FIG. 18 shows that very long-chain PUFA pretreatment inhibited cell death due to glutamate exposure in primary hippocampal cell cultures.

FIG. 19 shows that very long-chain PUFA pretreatment promoted healthy mitochondrial function when measured after glutamate-induced neurotoxicity in primary hippocampal cell culture.

FIG. 20 shows that very long-chain PUFA pretreatment generally inhibited cell death due to hypoxia in primary hippocampal cells, but these effects were not as robust as those observed after glutamate exposure.

FIG. 21 shows that very long-chain PUFA pretreatment generally promoted mitochondrial health after hypoxia in primary hippocampal cells, but these effects were not as robust as those observed after glutamate exposure.

FIG. 22 shows that pretreatment with the very long-chain PUFA 28:4n6 inhibited cell death due to either glutamate exposure or hypoxia in primary hippocampal cell cultures, but did not alter mitochondrial health overall.

### DESCRIPTION OF EMBODIMENTS

### Synthesis of VLC PUFAs

In one aspect, the invention provides chemical synthesis routes for the preparation of VLC-PUFAs starting with a source of docosahexaenoic acid (DHA) or docosapentaenoic acid (DPA). In one embodiment, DHASCO-T and DHASCO-S oils (Martek Biosciences Corporation, Columbia, Md.) are used as the source of the DHA and DPA starting materials. After generating ethyl esters of DHA and DPAn6, these ethyl esters are reduced to intermediate DHA and DPA alcohols that, on chemical oxidation, produce key intermediate aldehydes, such as docosahexaen-1-al. These aldehydes are coupled with saturated extender reagents to provide the VLC PUFAs. The intermediate DHA and DPA alcohols, on chemical halogenations produce intermediate halides such as docosahexaen-1-bromide. These bromides are coupled with saturated extender reagents to provide the VLC PUFAs.

### Transesterification

An initial step in the production of very long chain polyunsaturated fatty acids of the invention is obtaining an ethyl ester of a fatty acid starting material. Several fatty acid ethyl esters are available commercially, but specifically desired ethyl esters may optionally be obtained by transesterification of a suitable source of PUFAs. Transesterification methods of the present invention involve reacting a source of PUFA residues in the presence of an alcohol and a base to produce esters of the PUFAs.

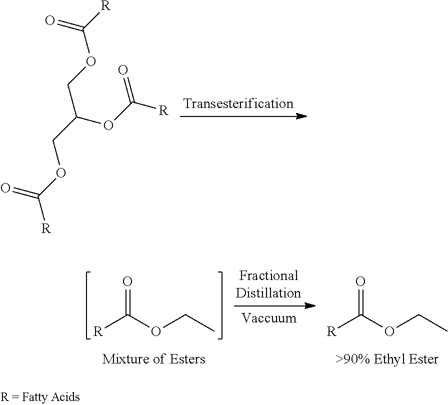

One source of PUFAs are compositions containing triglycerides having PUFA residues. Suitable triglycerides contain at least one PUFA. In some embodiments, the PUFA has a chain length of at least 18 carbons. In some embodiments, the PUFA can be docosahexaenoic acid C22:6 n-3 (DHA), omega-3 docosapentaenoic acid C22:5 n-3 (DPA), omega-6 docosapentaenoic acid C22:5 n-6 (DPA), arachidonic acid C20:4 n-6 (ARA), eicosapentaenoic acid C20:5 n-3 (EPA), stearidonic acid (SDA), linolenic acid (LLA), alpha linolenic acid (ALA), gamma linolenic acid (GLA), conjugated linolenic acid (CLA) or mixtures thereof. The PUFAs can also be present in any of the common forms found in natural lipids including but not limited to triacylglycerols, diacylglycerols, monoacylglycerols, phospholipids, free fatty acids, ceramides, sphingomyelins and other glycolipid forms as well as in natural or synthetic derivative forms of these fatty acids (e.g. calcium salts of fatty acids, and the like). Reference to an oil or other composition comprising triglycerides having PUFA residues, as used in the present invention, can refer to either a composition comprising triglycerides having only a single type of PUFA residue such as DHA or a composition comprising triglycerides having a mixture of more than one type of PUFA residues such as more than one of DHA, EPA and ARA. The following scheme shows the transesterification methods using a mixture of DHA and DPA PUFAs, such as DHASCO-S™ oil (Martek, Columbia, Md.).

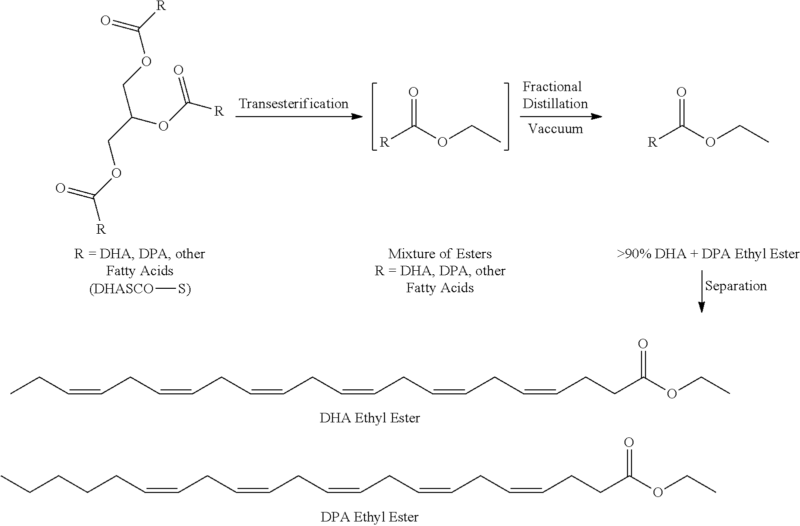

Compositions comprising triglycerides having PUFA residues can be obtained from, or derived from, any suitable source, such as a plant (including oilseeds), a microorganism, an animal, or mixtures of the foregoing. The microorganisms can be algae, bacteria, fungi or protists. Microbial sources and methods for growing microorganisms comprising nutrients and/or PUFAs are known in the art (Industrial Microbiology and Biotechnology, 2nd edition, 1999, American Society for Microbiology). For example, the microorganisms can be cultured in a fermentation medium in a fermentor. Oils produced by microorganisms can be used in the methods and compositions of the present invention. In some embodiments, organisms include those selected from the group consisting of golden algae (such as microorganisms of the kingdom Stramenopiles), green algae, diatoms, dinoflagellates (such as microorganisms of the order Dinophyceae including members of the genus Crypthecodinium such as, for example, Crypthecodinium cohnii), yeast, and fungi of the genera Mucor and Mortierella, including but not limited to Mortierella alpina and Mortierella sect. schmuckeri. Members of the microbial group Stramenopiles include microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Olliicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), Chlamydomyxa* (species include labyrinthuloides, montana). (*=there is no current general consensus on the exact taxonomic placement of these genera).

Suitable microorganisms include those capable of producing lipids comprising omega-3 and/or omega-6 polyunsaturated fatty acids, and in particular microorganisms that are capable of producing oils containing DHA, DPA, EPA or ARA will be described. More particularly, the microorganisms may be algae, such as Thraustochytrids of the order Thraustochytriales, including Thraustochytrium (including Ulkenia) and Schizochytrium and including Thraustochytriales which are disclosed in U.S. Pat. Nos. 5,340,594 and 5,340,742, both issued to Barclay, each of which are incorporated herein by reference in their entirety. More preferably, the microorganisms are selected from the group consisting of microorganisms having the identifying characteristics of ATCC number 20888, ATCC number 20889, ATCC number 20890, ATCC number 20891 and ATCC number 20892. Since there is some disagreement among experts as to whether Ulkenia is a separate genus from the genus Thraustochytrium, for the purposes of this application, the genus Thraustochytrium will include Ulkenia. Also preferred are strains of Mortierella schmuckeri (e.g., including ATCC 74371) and Mortierella alpina. Also preferred are strains of Crypthecodinium cohnii, including microorganisms having the identifying characteristics of ATCC Nos. 30021, 30334-30348, 30541-30543, 30555-30557, 30571, 30572, 30772-30775, 30812, 40750, 50050-50060, and 50297-50300. Oleaginous microorganisms are also useful. As used herein, “oleaginous microorganisms” are defined as microorganisms capable of accumulating greater than 20% of the dry weight of their cells in the form of lipids. Genetically modified microorganisms that produce PUFA-containing oils are also suitable for the present invention. These can include naturally PUFA-producing microorganisms that have been genetically modified as well as microorganisms that do not naturally produce PUFAs but that have been genetically modified to do so.

Suitable organisms can be obtained from a number of available sources, including by collection from the natural environment. For example, the American Type Culture Collection currently lists many publicly available strains of microorganisms identified above. As used herein, any organism, or any specific type of organism, includes wild strains, mutants, or recombinant types. Growth conditions in which to culture or grow these organisms are known in the art, and appropriate growth conditions for at least some of these organisms are disclosed in, for example, U.S. Pat. No. 5,130,242, U.S. Pat. No. No. 5,407,957, U.S. Pat. No. 5,397,591, U.S. Pat. No. 5,492,938, U.S. Pat. No. 5,711,983 and U.S. Pat. No. 6,607,900, all of which are incorporated herein by reference in their entirety. When microbial oils are used, the microorganisms are cultured in an effective medium, herein defined as any medium capable of promoting oil production. Preferably, the effective medium also promotes rapid microbial growth. The microorganisms can be cultured in conventional fermentation modes, which include, but are not limited to, batch, fed-batch, and continuous.

Another source of oils suitable for the transesterification methods of the present invention includes a plant source, such as oilseed plants. PUFA-producing plants, in alternate embodiments, can include those genetically engineered to express genes that produce PUFAs and those that produce PUFAs naturally. Such genes can include genes encoding proteins involved in the classical fatty acid synthase pathways, or genes encoding proteins involved in the PUFA polyketide synthase (PKS) pathway. The genes and proteins involved in the classical fatty acid synthase pathways, and genetically modified organisms, such as plants, transformed with such genes, are described, for example, in Napier and Sayanova, Proceedings of the Nutrition Society (2005), 64:387-393; Robert et al., Functional Plant Biology (2005) 32:473-479; or U.S. Patent Application Publication 2004/0172682. The PUFA PKS pathway, genes and proteins included in this pathway, and genetically modified microorganisms and plants transformed with such genes for the expression and production of PUFAs are described in detail in: U.S. Pat. No. 6,140,486, U.S. Pat. No. 6,566,583; U.S. Patent Application Publication No. 20020194641, U.S. Pat. No. 7,211,418, U.S. Patent Application Publication No. 20050100995A1, U.S. Patent Application Publication No. 20070089199, PCT Publication No. WO 05/097982, and U.S. Patent Application Publication No. 20050014231, each of which is incorporated herein by reference in its entirety.

Oilseed crops suitable for use in the present invention include soybeans, corn, rice, safflower, sunflower, canola, flax, peanut, mustard, rapeseed, chickpea, cotton, lentil, white clover, olive, palm oil, borage, evening primrose, linseed, and tobacco that have been genetically modified to produce a PUFA.

Alcohols suitable for use in the present invention include any lower alkyl alcohol containing from 1 to 6 carbon atoms (i.e., a C₁₋₆ alkyl alcohol). Use of lower alkyl alcohols in the methods of the present invention produces lower alkyl esters of the PUFAs. For example, the use of ethanol produces ethyl esters. In certain embodiments, the alcohol is methanol or ethanol. In these embodiments, the PUFA esters produced are a methyl ester and an ethyl ester of the PUFA, respectively. In processes of the present invention, the alcohol typically comprises between about 25 wt. % and about 50 wt. %; between about 30 wt. % and about 45 wt. %, or between about 35 wt. % and about 40 wt. % of the mixture of the composition, the alcohol and the base. In some embodiments, the alcohol comprises about 38 wt. % of the mixture of the composition, the alcohol and the base. In certain embodiments, the composition and the base can be added to either pure ethanol or pure methanol. In general, the amount of alcohol used may vary with the solubility of the oil or composition containing triglycerides having PUFA residues in the alcohol.

Any base known in the art to be suitable for use as a reactant may be used in the present invention. Bases of the formula RO-M, wherein M is a monovalent cation and RO is an alkoxide of a C₁₋₆ alkyl alcohol are particularly suited for the present invention. Examples of suitable bases include elemental sodium, sodium methoxide, sodium ethoxide, potassium methoxide, and potassium ethoxide. In some embodiments, the base is sodium ethoxide. In processes of the present invention, the base is typically added in an amount of between about 0.5 and about 1.5 molar equivalents of triglycerides, between about 0.7 and about 1.4 molar equivalents of triglycerides, between about 0.9 and about 1.3 molar equivalents of triglycerides, or between about 1.0 and about 1.2 molar equivalents of triglycerides to the reaction step with the composition and the alcohol. In certain embodiments, the base is typically added in an amount of about 0.1, 0.2, 0.3, 0.4, 0.5, 0.6, 0.7, 0.8, 0.9, 0.95, 1.0, 1.01, 1.02, 1.03, 1.04, 1.05, 1.10, 1.15, 1.2, 1.3, 1.4, or 1.5 molar equivalents of triglycerides to the reaction step with the composition and the alcohol. In some embodiments, the base is added in an amount of 1.04 molar equivalents of triglycerides to the reaction step with the composition and the alcohol.

The composition comprising triglycerides having polyunsaturated fatty acid residues, the alcohol and the base are reacted together at a temperature and for an amount of time that allows the production of an ester between the fatty acid residues and the alcohol. Suitable reaction times and temperatures may be determined by one of skill in the art to produce an ester. Without intending to be bound by theory, the PUFA residues are believed to be cleaved from the glycerol backbone of the triglyceride and esters of each PUFA residue are formed during the reacting step. In certain embodiments, the step of reacting the composition in the presence of an alcohol and a base is performed at a temperature from about 60° C. to about 120° C., from about 70° C. to about 110° C., from about 75° C. to about 100° C., or from about 80° C. to about 90° C. In further embodiments, the step of reacting the composition in the presence of an alcohol and a base is performed at a temperature of about 75° C., 80° C., 85° C., 90° C., or 95° C. In some embodiments, the step of reacting the composition in the presence of an alcohol and a base is performed for a time from about 2 hours to about 12 hours, from about 3 hours to about 11 hours, from about 4 hours to about 10 hours, from about 5 hours to about 9 hours, or from about 6 hours to about 8 hours. In certain embodiments, the step of reacting the composition in the presence of an alcohol and a base is performed for about 5.5, 6, 6.5, 7, 7.5, 8, or 8.5 hours

In one embodiment, the step of reacting the oil composition, alcohol and base may be conducted by refluxing the components to produce the PUFA esters. In additional embodiments, the step of reacting the oil composition may be carried out at a temperature that does not result in the refluxing of the reaction components. For example, carrying out the step of reacting the oil composition under pressures greater than atmospheric pressure can increase the boiling point of the solvents present in the reaction mixture. Under such conditions, the reaction can occur at a temperature at which the solvents would boil at atmospheric pressure, but would not result in the refluxing of the reaction components. In some embodiments, the reaction is conducted at a pressure from about 5 to about 20 pounds per square inch (psi); from about 7 to about 15 psi; or from about 9 to about 12 psi. In certain embodiments, the reaction is conducted at a pressure of about 7, 8, 9, 10, 11, or 12 psi. Reactions conducted under pressure may be carried out at the reaction temperatures listed above. In some embodiments, reactions conducted under pressure may be carried out at about 70° C., 75° C., 80° C., 85° C., or 90° C.

The reaction mixture comprising PUFA esters can be further processed to obtain the PUFA esters from the mixture. For example, the mixture may be cooled, diluted with water, and the aqueous solution extracted with a solvent such as hexane to produce a composition comprising PUFA esters. Techniques for washing and/or extracting crude reaction mixtures are known in the art.

In one embodiment of the present invention, PUFA esters are separated from the reaction mixture by distilling the composition to recover a fraction comprising the ester of the polyunsaturated fatty acid. In this manner, a targeted fraction of the reaction mixture including PUFA esters of interest can be separated from the reaction mixture and recovered.

In certain embodiments, the distillation is performed under vacuum. Without being bound by theory, distillation under vacuum allows the distillation to be accomplished at a lower temperature than in the absence of a vacuum and thus may prevent the degradation of the esters. Typical distillation temperatures range from about 120° C. to about 170° C. In some embodiments, the step of distilling is performed at a temperature of less than about 180° C., less than about 175° C., less than about 170° C., less than about 165° C., less than about 160° C., less than about 155° C., less than about 150° C., less than about 145° C., less than about 140° C., less than about 135° C., or less than about 130° C. Typical pressures for vacuum distillation range from about 0.1 mm Hg to about 10 mm Hg. In some embodiments, the pressure for vacuum distillation is about 0.1, 0.5, 1, 1.5, 2, 2, 5, 3, 3.5, or 4 mm Hg.

These methods may be used to produce compositions that contain a high percentage of PUFA esters. For example, such compositions can contain between about 50 wt. % and about 100 wt. % of an ester of a PUFA, and in other embodiments, the composition can comprise at least about 80 wt. %, at least about 85 wt. %, at least about 90 wt. %, at least about 95 wt. %, esters of a PUFA.

After the production of the ethyl esters is complete, any residual water may be removed from the esters by, for example, washing the solvent with brine and/or passing the solvent over an anhydrous salt (e.g., sodium sulfate). The solution then preferably is concentrated by, for example, evaporating a portion of the solvent.

The ethyl esters obtained commercially or produced by transesterification are reduced to produce intermediate alcohols. The reduction of the esters to alcohols may take place under any suitable reducing conditions known to those of skill in the art. In a particular embodiment, the reduction takes place at about 0° C. in the presence of lithium aluminum hydroxide (LAH) and tetrahydrofuran (THF). This reduction is shown in the following scheme using the example of DHA ethyl ester.

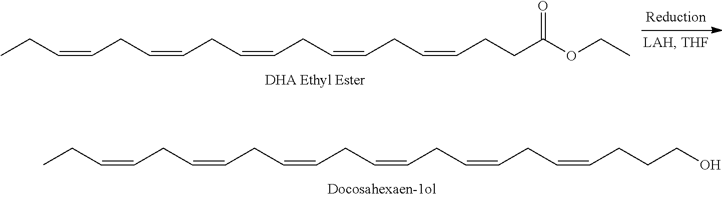

In one embodiment, the intermediate alcohol is converted to a bromide intermediate, which is then coupled with a saturated magnesium Grignard extender reagent to produce an elongated VLC-PUFA of any desired length, the length being determined by the extender reagent used in this elongation reaction. In a particular embodiment, this extension reaction is conducted at 60-65° C. in the presence of the catalyst lithium chloride-copper chloride in a mixture of N-methylpyrrolidone (NMP) and anhydrous tetrahydrofuran (THF). The resulting protected alcohol is de-protected in toluenesulfonic acid (TsOH) and methanol at room temperature, and washed with aqueous sodium bicarbonate. Optionally, the resulting alcohol may be oxidized to produce the free acid. Preferably, this is accomplished using the strong oxidant Periodic acid (H₅IO₆/CrO₃) followed by a wash of cold acetonitrile and water. This extension reaction is illustrated in the following reaction scheme using the example of docosahexaen-1-ol (i.e. the alcohol derived from DHA) and 6-tetrahydropyranyloxyhexyl magnesium chloride to form a C28 intermediate. One of skill in the art will appreciate that a different halide salt (such as, for example, 6-tetrahydropyranyloxyhexyl magnesium bromide) of the 6-tetrahydropyranyloxyhexyl can be successfully used in this synthesis reaction.

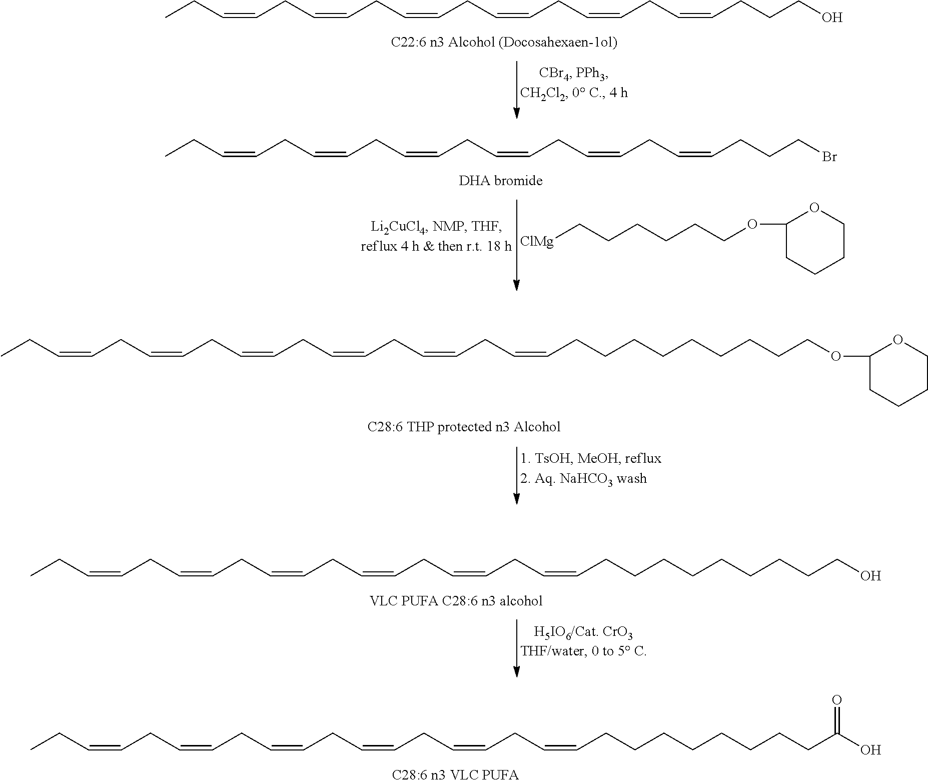

In another embodiment, the intermediate PUFA alcohols, like docosahexaen-1-ol (i.e. the alcohol derived from DHA), are oxidized to produce intermediate aldehydes. The oxidation of the alcohols to aldehydes may take place under any mild oxidizing conditions known to those of skill in the art. In a particular embodiment, the oxidation takes place at about −78° C. in the presence of dimethyl sulfoxide (DMSO), oxalyl chloride and triethylamine (Et₃N). This oxidation is shown in the following reaction scheme using the example of a DHA alcohol to form docosahexaen-1-al.

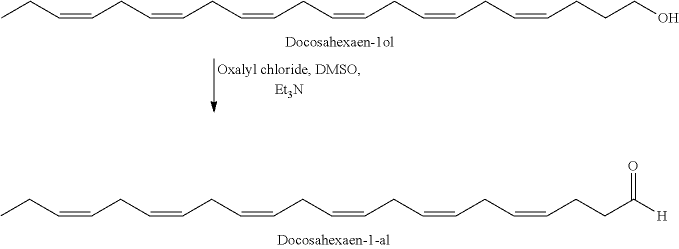 [SMILES: CC/C=C\C/C=C\C/C=C\C/C=C\C/C=C\C/C=C\CCCO ; CC/C=C\C/C=C\C/C=C\C/C=C\C/C=C\C/C=C\CCC=O]

In another embodiment, the intermediate aldehydes are coupled with a saturated zinc extender reagent to produce elongated VLC PUFAs of any desired length, the length being determined by the extender reagent used in this elongation reaction. In a particular embodiment, this extension reaction is conducted at about 0° C. in the presence of the catalyst titanium isopropoxide (Ti (i-OPr)₄) in anhydrous tetrahydrofuran (THF). This extension reaction is illustrated in the following reaction scheme using docosahexaen-1-al (i.e. the aldehyde derived from DHA) and 6-Ethoxy-6-oxohexylzinc bromide to form a C28 intermediate.

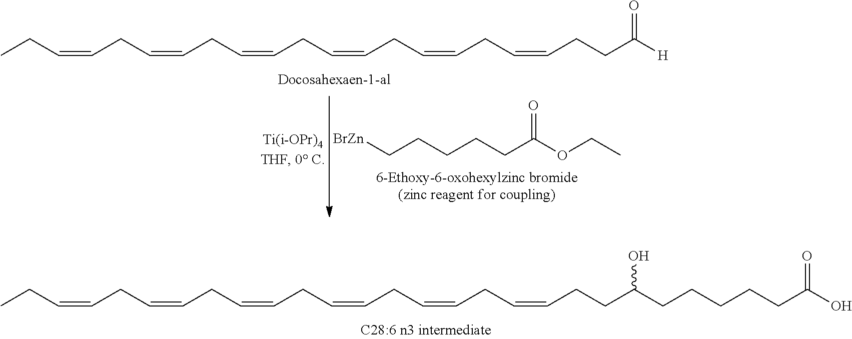

In a related embodiment, the intermediate aldehydes are coupled with a saturated zinc extender reagent in the presence of a cupurate salt, to provide the elongated VLC PUFAs of any desired length, the length being determined by the extender reagent used in this elongation reaction. In a particular embodiment, this extension reaction is conducted at 0° C. in the presence of CuCN.2 LiC, titanium isopropoxide (Ti(i-OPr)₄) in tetrahydrofuran (THF). This extension reaction is illustrated in the following reaction scheme, which, similar to the elongation scheme shown above, uses docosahexaen-1-al (i.e. the aldehyde derived from DHA) and 6-Ethoxy-6-oxohexylzinc bromide to form a C28 intermediate, but includes the use of a cupurate catalyst, resulting in an ethyl ester intermediate.

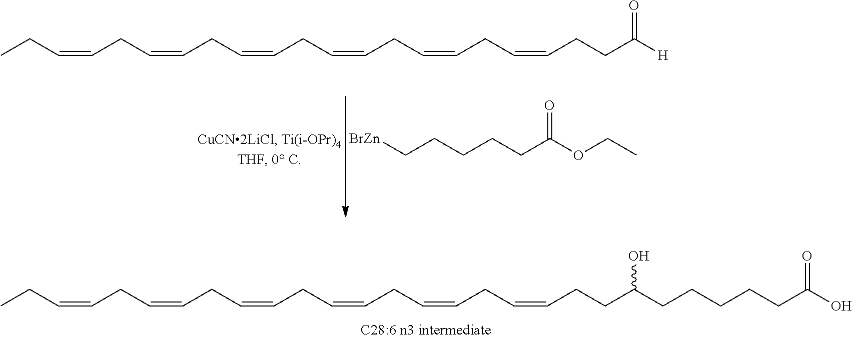

In an alternative embodiment, an extended carbon chain ester intermediate is formed via halogenation of the primary alcohol. The halogenated hydrocarbon is coupled with an aldehyde of any desired length to form the elongated ester. In one embodiment, the halogenation of the primary alcohol is conducted at about 0° C. using carbon tetrabromide, in the presence of triphenyl phosphene and methylene chloride. As described above, the polyunsaturated fatty acid ester starting materials may be obtained commercially or prepared by transesterifiction of a source of mono-, di- or triglycerides. This coupling technique is illustrated in the following reaction scheme using the DHA ethyl ester starting material to form the C32 ester intermediate.

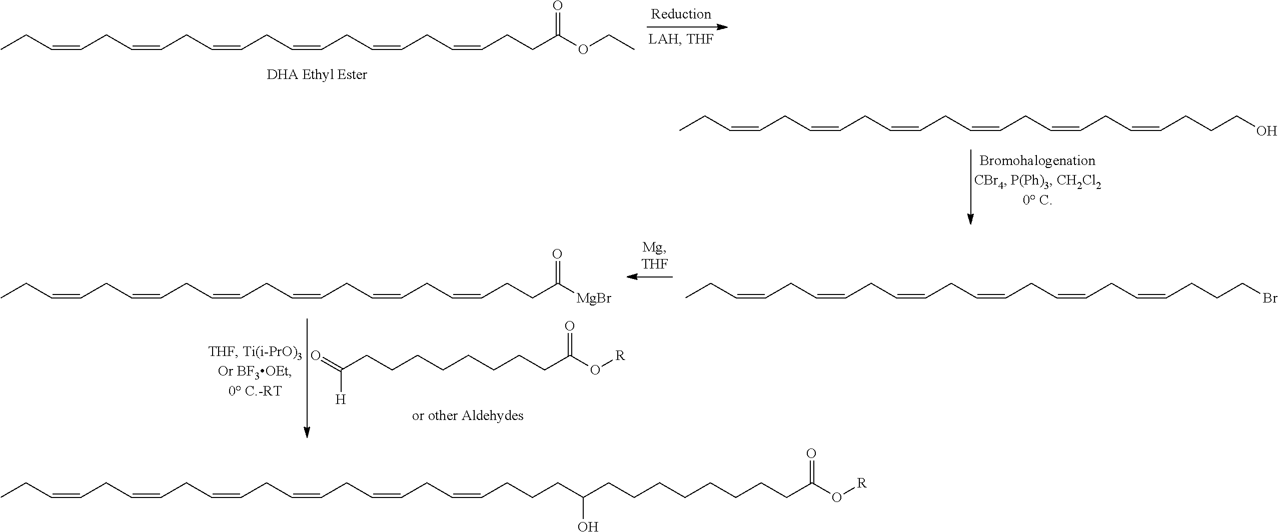

In a related embodiment, a zinc extending reagent is coupled with the halogenated hydrocarbon, which was created by halogenation of the reduced alcohol. The halogenated hydrocarbon and the zinc extending reagent are coupled in the presence of a palladium catalyst to form an ester of desired length, followed by hydrolysis of the ester to form an elongated polyunsaturated fatty acid of the desired length. This embodiment is illustrated in the following reaction scheme using DHA coupling with 6-Ethoxy-6-oxohexylzinc bromide followed by ester hydrolysis to form the C32 VLC PUFA.

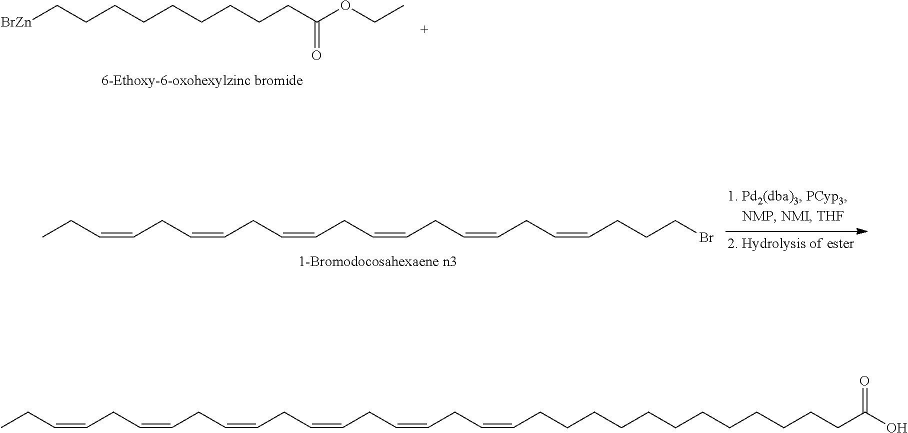

The ester intermediates of any of these embodiments may be converted to a VLC PUFA of the present invention by removing any alcohol and the ester moiety from the ester intermediate to form a fatty acid of at least 28 carbons in length. The reduction of the secondary alcohol in the presence of diphenylsilane, followed by hydrolysis is shown in the following reaction scheme.

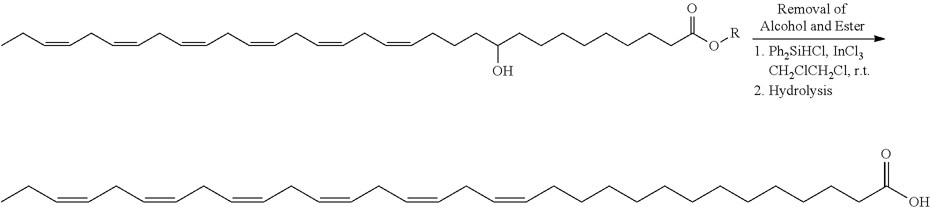

Using any of the reactions described above, VLC PUFAs of the present invention are prepared and may be further purified for use in research or for administration to mammals for therapeutic purposes in a purified form or in pharmaceutically acceptable compositions.

All 26-carbon to 42-carbon (C26-C42) n-3 and n-6 fatty acids may be prepared using the synthesis methods of the present invention. In some embodiments, the VLC PUFAs of the present invention that are prepared using the synthesis methods of the present invention include:

C28:3, 4, 5 & 6 n3 and n6 PUFAs

C32:4, 5 & 6 n3 and n6 PUFAs

C34:4, 5 & 6 n3 and n6 PUFAs

C36:4, 5 & 6 n3 and n6 PUFAs

C38:4, 5 & 6 n3 and n6 PUFAs

C40:4, 5 & 6 n3 and n6 PUFAs, and

C42:4, 5 & 6 n3 and n6 PUFAs.

### Compositions of the Invention

For the purposes of this application, very long chain polyunsaturated fatty acids (VLC-PUFAs) are defined as fatty acids of 26 and more carbon chain length, and are preferably fatty acids of 32 or more carbon chain length, and are more preferably fatty acids of 32-42 carbon chain length, containing 3 or more double bonds.

The terms “polyunsaturated fatty acid” and “PUFA” include not only the free fatty acid form, but other forms as well, such as the triacylglycerol (TAG) form, the phospholipid (PL) form, the ethyl ester form and other esterified forms.

As used herein, the term “lipid” includes phospholipids; free fatty acids; esters of fatty acids; triacylglycerols; diacylglycerides; monoacylglycerides; lysophospho lipids; soaps; phosphatides; ceramides; sphingomyelins and other glycolipids, sterols and sterol esters; carotenoids; xanthophylls (e.g., oxycarotenoids); hydrocarbons; and other lipids known to one of ordinary skill in the art.

As used herein, the term “analog” refers to a chemical compound that is structurally similar to another compound but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. For example, the reference compound can be a reference VLC-PUFA, and an analog is a substance possessing a chemical structure or chemical properties similar to those of the reference VLC-PUFA.

The terms “substituted,” “substituted derivative” and “derivative,” when used to describe a VLC-PUFA of the present invention, means that at least one hydrogen bound to the unsubstituted compound is replaced with a different atom or a chemical moiety. Examples of substituents include, but are not limited to, hydroxy, alkyl, halogen, nitro, cyano, heterocycle, aryl, heteroaryl, amino, amide, ester, ether, carboxylic acid, thiol, thioester, thioether, sulfoxide, sulfone, carbamate, peptidyl, PO₃H₂, and mixtures thereof. Although a derivative has a similar physical structure to the parent compound, the derivative may have different chemical and/or biological properties than the parent compound. Such properties can include, but are not limited to, increased or decreased activity of the parent compound, new activity as compared to the parent compound, enhanced or decreased bioavailability, enhanced or decreased efficacy, enhanced or decreased stability in vitro and/or in vivo, and/or enhanced or decreased absorption properties. Examples of derivatives include esters, aldehydes and alcohols of the VLC-PUFAs of the present invention. An example includes an ethyl ester of the VLC-PUFAs of the invention.

In general, the term “biologically active” indicates that a compound has at least one detectable activity that has an effect on the metabolic or other processes of a cell or organism, as measured or observed in vivo (i.e., in a natural physiological environment) or in vitro (i.e., under laboratory conditions). The therapeutically active VLC-PUFAs include the free, esterified, alcohol, oxygenated and aldehyde forms of these compounds.

One embodiment of the present invention relates to any compositions or formulations or products containing VLC-PUFAs, or analogs or derivatives thereof, as well as oils or other compositions or formulations or products that have been enriched for any VLC-PUFA or analogs or derivatives thereof, and particularly for any VLC-PUFA derived chemically from DHA, EPA, DPAn-6, ARA, DPAn-3 or DTAn-6, and more particularly, for any docosanoid, and even more particularly, for any PUFA derived from DHA or DPA or combinations of DHA and DPA. The present invention also relates to any oils or other compositions or formulations or products in which such VLC-PUFAs are stabilized or retained in the oils or compositions to improve the quantity, quality or stability of the PUFAs in the oil or composition, and/or to improve the absorption, bioavailability, and/or efficacy of the PUFAs contained in oils or compositions.

A variety of DHA-, DPA- and EPA-derived PUFAs having anti-inflammatory activity, anti-proliferative activity, antioxidant activity, neuroprotective or vasoregulatory activity (Ye et al, 2002) are known. Additionally, as described above, VLC-PUFAs are expected to have efficacy in the prevention and treatment of disorders of the retina, including age related maculopathies, male fertility disorders, skin disorders, disorders of neurological development and neurodegenerative diseases. Such PUFAs and VLC-PUFAs are encompassed by the present invention, particularly in embodiments where such VLC-PUFAs are enriched in oils and compositions, preferably using the synthetic methods and processing steps of the present invention, in combination with DHA-, DPA- and EPA-derived PUFAs. Such compositions can be used in any therapeutic, nutritional (including nutraceutical), cosmetic, or other application. Therefore, the present invention encompasses isolated, semi-purified and purified VLC-PUFAs, as well as sources of PUFAs including synthesized and natural sources (e.g., oils or plants and portions thereof), and includes a VLC-PUFA useful in the present invention synthesized by processing steps as described herein. The VLC-PUFAs and PUFAs in these compositions may be present as ethyl esters.

VLC-PUFAs may have either pro-inflammatory or anti-inflammatory properties. According to the present invention, pro-inflammatory properties are properties (characteristics, activities, functions) that enhance inflammation in a cell, tissue or organism, and anti-inflammatory properties are properties that inhibit such inflammation. Inflammation in cells, tissues and/or organisms can be identified by a variety of characteristics including, but not limited to, the production of “proinflammatory” cytokines (e.g., interleukin-1a (IL-1α), IL-1β, tumor necrosis factor-α (TNFα), IL-6, IL-8, IL-12, macrophage inflammatory protein-1α (MIP-1α), macrophage chemotactic protein-1 (MCP-1; also known as macrophage/monocyte chemotactic and activating factor or monocyte chemoattractant protein-1) and interferon-γ (IFN-γ)), eicosanoid production, histamine production, bradykinin production, prostaglandin production, leukotriene production, fever, edema or other swelling, and accumulation of cellular mediators (e.g., neutrophils, macrophages, lymphocytes, etc.) at the site of inflammation.

Therefore, one embodiment is a method to prevent or reduce at least one symptom of inflammation or neurodegeneration or retinal degeneration or macular degeneration or male infertility in an individual in need of such therapy, comprising administering to an individual at risk of, diagnosed with, or suspected of having inflammation or neurodegeneration or a retinal degeneration or macular degeneration or male infertility or a condition or disease related thereto, a pharmaceutical composition comprising a VLC-PUFA. The isolated VLC-PUFA of this method may be one of C28:4, 5 & 6 n3 and n6 PUFAs, C32:4, 5 & 6 n3 and n6 PUFAs, C34:4, 5 & 6 n3 and n6 PUFAs; and, C36:4, 5 & 6 n3 and n6 PUFAs. The isolated VLC-PUFA may be provided in one of the following forms: as triglyceride containing the long chain fatty acid, as a phospholipid or glycolipid containing the long chain fatty acid, as a free fatty acid, or as an ethyl or methyl ester of the VLC-PUFA.

Another embodiment is a method of the dietary management of the levels of a VLC-PUFA in an individual in need of such dietary management. The VLC-PUFA of this method may be one of C28:4, 5 & 6 n3 and n6 PUFAs, C32:4, 5 & 6 n3 and n6 PUFAs, C34:4, 5 & 6 n3 and n6 PUFAs; and, C36:4, 5 & 6 n3 and n6 PUFAs. The isolated VLC-PUFA used to modify the levels of a VLC-PUFA in the individual may be provided in one of the following forms: as triglyceride containing the long chain fatty acid, as a phospholipid or glycolipid containing the long chain fatty acid, as a free fatty acid, or as an ethyl or methyl ester of the VLC-PUFA.-161

Another embodiment is a method of treating a neurodegenerative disease in an individual comprising administering an effective amount of a VLCPUFA to an individual in need thereof. In this embodiment, the neurodegenerative disease may be one of Alzheimer's disease, mild cognitive impairment, age related cognitive decline, Huntington's disease, Parkinson's disease, and Pick's disease.

Another embodiment is a method of treating an individual suffering from or at risk of traumatic brain injury comprising administering an effective amount of a VLCPFU to the individual in need thereof.

Another embodiment is a method of treating an individual suffering from spinal cord injury comprising administering an effective amount of a VLCPUFA to the individual in need thereof.

Another embodiment is a method of treating stroke in an individual comprising administering an effective amount of a VLC-PUFA to the individual in need thereof.

Another embodiment is a method of treating macular degeneration degenerative eye condition selected from the group consisting of macular degeneration, and Stargardt's disease in an individual comprising administering an effective amount of a VLC-PUFA to the individual in need thereof.

Another embodiment is a method of treating infertility related to a disorder in sperm motility or disorders of the testes in an individual comprising administering an effective amount of a VLC-PUFA to the individual in need thereof.

Another embodiment is a method of the dietary management of a neurodegenerative disease selected from the group consisting of Alzheimer's disease, mild cognitive impairment, age related cognitive decline, Huntington's disease, Parkinson's disease, and Pick's disease in an individual comprising administering an effective amount of a VLC-PUFA to the individual in need thereof.

Another embodiment is a method of clinical dietary management of a neurodegenerative disease selected from the group consisting of Alzheimer's disease, mild cognitive impairment, age related cognitive decline, Huntington's disease, Parkinson's disease, and Pick's disease in an individual comprising administering an effective amount of a VLC-PUFA to the individual in need thereof.

Another embodiment is a method of clinical dietary management of the metabolic processes associated with a neurodegenerative disease selected from the group consisting of Alzheimer's disease, mild cognitive impairment, age related cognitive decline, Huntington's disease, Parkinson's disease, Pick's disease in an individual comprising administering an effective amount of a VLC-PUFA to the individual in need thereof.

Another embodiment is a method of the dietary management of traumatic brain injury or spinal cord injury in an individual comprising administering an effective amount of a VLC-PUFA to the individual in need thereof.

Another embodiment is a method of clinical dietary management of traumatic brain injury or spinal cord injury in an individual comprising administering an effective amount of a VLC-PUFA to the individual in need thereof.

Another embodiment is a method of clinical dietary management of the metabolic processes associated with traumatic brain injury or spinal cord injury in an individual comprising administering an effective amount of a VLC-PUFA to the individual in need thereof.

Another embodiment is a method of the clinical dietary management of stroke in an individual comprising administering an effective amount of a VLC-PFUA to the individual in need thereof.

Another embodiment is a method of clinical dietary management of stroke in an individual comprising administering an effective amount of a VLC-PFUA to the individual in need thereof.

Another embodiment is a method of clinical dietary management of the metabolic processes associated with stroke comprising administering an effective amount of a VLC-PFUA to the individual in need thereof.

Another embodiment is a method of the dietary management of spinal cord injury in an individual comprising administering an effective amount of a VLC-PUFA to the individual in need thereof.

Another embodiment is a method of clinical dietary management of spinal cord injury in an individual comprising administering an effective amount of a VLC-PUFA to the individual in need thereof.

Another embodiment is a method of clinical dietary management of the metabolic processes associated with spinal cord injury comprising administering an effective amount of a VLC-PUFA to the individual in need thereof.

Another embodiment is a method of the dietary management of retinal health in an individual comprising administering an effective amount of a VLC-PUFA to the individual in need thereof.

Another embodiment is a method of clinical dietary management of retinal health in an individual comprising administering an effective amount of a VLC-PUFA to the individual in need thereof.

Another embodiment is a method of clinical dietary management of the metabolic processes associated with retinal health comprising administering an effective amount of a VLC-PUFA to the individual in need thereof.

Another embodiment is a method of the dietary management of a cardiovascular disorder or disease, or condition associated with cardiovascular disease in an individual comprising administering an effective amount of a VLC-PUFA to the individual in need thereof.

Another embodiment is a method of clinical dietary management of a cardiovascular disorder or disease, or condition associated with cardiovascular disease in an individual comprising administering an effective amount of a VLC-PUFA to the individual in need thereof.

Another embodiment is a method of clinical dietary management of the metabolic processes associated with a cardiovascular disorder or disease, or condition associated with cardiovascular disease in an individual comprising administering an effective amount of a VLC-PUFA to the individual in need thereof.

Another embodiment is a method of the dietary management of inflammation or a disease or condition associated with inflammation in an individual comprising administering an effective amount of a VLC-PUFA to the individual in need thereof.

Another embodiment is a method of clinical dietary management of inflammation or a disease or condition associated with inflammation in an individual comprising administering an effective amount of a VLC-PUFA to the individual in need thereof.

Another embodiment is a method of clinical dietary management of the metabolic processes associated with inflammation or a disease or condition associated with inflammation in an individual comprising administering an effective amount of a VLC-PUFA to the individual in need thereof.

Another embodiment is a method of the dietary management of degenerative eye condition selected from the group consisting of macular degeneration, and Stargardt's disease in an individual comprising administering an effective amount of a VLC-PUFA to the individual in need thereof.

Another embodiment is a method of clinical dietary management of degenerative eye condition selected from the group consisting of macular degeneration, and Stargardt's disease in an individual comprising administering an effective amount of a VLC-PUFA to the individual in need thereof.

Another embodiment is a method of clinical dietary management of the metabolic processes associated with degenerative eye condition selected from the group consisting of macular degeneration, and Stargardt's disease in an individual comprising administering an effective amount of a VLC-PUFA to the individual in need thereof.

Another embodiment is a method of the dietary management of fertility related to a disorder in sperm motility or disorders of the testes in an individual comprising administering an effective amount of a VLC-PUFA to the individual in need thereof.

Another embodiment is a method of clinical dietary management of fertility related to a disorder in sperm motility or disorders of the testes in an individual comprising administering an effective amount of a VLC-PUFA to the individual in need thereof.

Another embodiment is a method of clinical dietary management of the metabolic processes associated with fertility related to a disorder in sperm motility or disorders of the testes in an individual comprising administering an effective amount of a VLC-PUFA to the individual in need thereof.

In another embodiment an effective amount of a VLC-PUFA is administered to an individual to promote, improve, and/or develop brain, eye, and/or fertility health and or function.

In another embodiment an effective amount of a VLC-PUFA is administered to an individual to improve cognition.

Another embodiment is a method of preventing excitotoxicity to a nerve cell caused by excessive stimulation by the neurotransmitter glutamate comprising administering an effective amount of a VLC-PUFA to an individual in need of such pretreatment.

Another embodiment is a method of preventing hypoxic injury to a nerve cell comprising administering an effective amount of a VLC-PUFA to an individual in need of such pretreatment.

Another embodiment is a method of preventing stroke in an individual comprising administering an effective amount of a VLC-PUFA to an individual in need of such pretreatment.

Another embodiment is a method of promoting mitochondrial health in an individual comprising administering an effective amount of a VLC-PUFA to an individual in need of such pretreatment.

In these embodiments, the VLC-PUFA administered may be one of C34:6n3, C34:5n3, C:34:4n6, C28:6n3, C28:5n3 and 28:4n6. In a specific embodiment, the VLC-PUFA comprises C34:6n3. In another specific embodiment, the VLC-PUFA comprises C34:6n3. In another specific embodiment, the VLC-PUFA comprises C34:6n3. In another specific embodiment, the VLC-PUFA comprises C34:6n6. In another specific embodiment, the VLC-PUFA comprises C28:6n3. In another specific embodiment, the VLC-PUFA comprises C28:5n3. In another specific embodiment, the VLC-PUFA comprises C28:4n6.

It will be appreciated by those skilled in the art that VLC-PUFA compounds, analogs or derivatives of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-inflammatory activity, for example, using standard tests described herein, or using other similar tests which are well known in the art.

Prodrugs of any of the VLC-PUFAs described herein may be identified using routine techniques known in the art. Various forms of prodrugs are known in the art. For examples of such prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8, 1-38 (1992); d) H. Bundgaard, et al., Journal of Pharmaceutical Sciences, 77:285 (1988); and e) N. Kakeya, et al., Chem. Pharm. Bull., 32: 692 (1984), each of which is specifically incorporated herein by reference.

In addition, the invention also includes solvates, metabolites, and salts (preferably pharmaceutically acceptable salts) of compounds of any of the VLC-PUFAs described herein. The term “solvate” refers to an aggregate of a molecule with one or more solvent molecules. A “metabolite” is a pharmacologically active product produced through in vivo metabolism in the body or organism of a specified compound or salt thereof. Such products may result for example from the oxidation, reduction, hydrolysis, amidation, deamidation, esterification, deesterification, enzymatic cleavage, and the like, of the administered or produced compound. Accordingly, the invention includes metabolites of any of the VLC-PUFAs or analogs or derivatives described herein, including compounds produced by a process comprising contacting a compound of this invention with an organism for a period of time sufficient to yield a metabolic product thereof.

A “pharmaceutically acceptable salt” or “salt” as used herein, includes salts that retain the biological effectiveness of the free acids and bases of the specified compound and that are not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable sale. Examples of pharmaceutically acceptable salts include those salts prepared by reaction of the compounds of the present invention with a mineral or organic acid or an inorganic base, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitromenzoates, hydroxybenzoates, methoxybenzoates, phthalates, sulfonates, xylenesulfonates, pheylacetates, phenylpropionates, phenylbutyrates, citrates, lactates, .gamma.-hydroxybutyrates, glycollates, tartrates, methanesulfonates, propanesulfonates, naphthalene-1-sulfonates, naphthalene-2-sulfonates, and mandelates. Since a single compound of the present invention may include more than one acidic or basic moieties, the compounds of the present invention may include mono, di or tri-salts in a single compound.

If the VLC-PUFA compound is a base, the desired pharmaceutically acceptable salt may be prepared by any suitable method available in the art, for example, treatment of the free base with an acidic compound, particularly an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alphahydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the VLC-PUFA compound is an acid, the desired pharmaceutically acceptable salt may be prepared by any suitable method, for example, treatment of the free acid with an inorganic or organic base. Particular inorganic salts are those formed with alkali and alkaline earth metals such as lithium, sodium, potassium, barium and calcium. Particular organic base salts include, for example, ammonium, dibenzylammonium, benzylammonium, 2-hydroxyethylammonium, bis(2-hydroxyethyl)ammonium, phenylethylbenzylamine, dibenzylethylenediamine, and the like salts. Other salts of acidic moieties may include, for example, those salts formed with procaine, quinine and N-methylglusoamine, plus salts formed with basic amino acids such as glycine, ornithine, histidine, phenylglycine, lysine and arginine.

Oils containing VLC-PUFAs described herein may be formed as oils in their free and/or esterifed forms. In the esterified form, the VLC-PUFAs can be present in the triglyceride, diglyceride, monoglyceride, phospholipid, sterol ester and/or wax ester forms. Since the VLC-PUFAs have only been described previously in the free fatty acid form, the esterified forms represent forms of VLC-PUFAs, the presence of which can be enhanced, stabilized or retained in oils or compositions of the present invention. Preferably, the VLC-PUFAs present in these oils are esterified, and more preferably, the VLC-PUFAs in these oils are present as ethyl esters.

The oils and compositions according to the present invention may have concentrations of VLC-PUFAs, and analogs and derivatives thereof, that are at least 2×, at least 3×, at least 4×, at least 5×, at least 10×, at least 20×, at least 50×, at least 100×, at least 200×, at least 400×, at least 1,000×, or at least 5,000× higher (including any other increment of 1×, e.g., 20×, 21×, 22×, etc.) than the trace concentrations normally found in VLC-PUFA tissue sources that have been separated through the standard refining, bleaching, and deodorization process commonly used for edible oils. VLC-PUFA oils and compositions produced by the processes of the present invention will preferably contain at least 1 μg, at least 5 μg, at least 10 μg, at least 15 μg, at least 20 μg, at least 30 μg, at least 50 μg, at least 100 μg, at least 200 μg, at least 500 μg, at least 1,000 μg, at least 2,000 μg, at least 5,000 μg, at least 10,000 μg, or at least 50,000 μg or more of at least one or more ethyl esters of a VLC-PUFA per gram of oil (including any other increment in 0.1 μg increments). It is noted that through processing and purification of oils or compositions, the VLC-PUFA concentrations could actually be much higher (e.g., approaching 100%) during the production phase, although the oils and compositions would typically be diluted or titrated to the amounts described above, prior to being used in a nutritional, therapeutic, cosmetic or other process. Preferably the oils and compositions of the present invention are formulated to contain a concentration of VLC-PUFAs or analogs or derivatives thereof that enables easy measurement and administration of a therapeutically-effective amount of these compounds.

Preferably, the oils produced from the present invention are enriched with ethyl ester forms of VLC-PUFAs and may also include esters of DHA, and/or EPA and/or DPAn-3 and/or DPAn-6, and/or DTAn-6.

Oils, compositions, or pharmaceutical formulations (or any products) useful in the present invention preferably include VLC-PUFAs in an amount that is at least about 2 weight percent, or at least about 5 weight percent, or at least about 10 weight percent, or at least about 15 weight percent, or at least about 20 weight percent, or at least about 25 weight percent, or at least about 30 weight percent, or at least about 35 weight percent, or at least about 40 weight percent, or at least about 45 weight percent, or at least about 8050 weight percent, and so on, in increments of 1 weight percent (i.e., 2, 3, 4, 5, . . . ) up to or at least about 90 weight percent or more, or up to at least about 95 weight percent or higher of the total lipids in the oil, composition of formulation.

In another embodiment, the oil, composition, formulation or other product comprises a therapeutically-effective amount of a VLC-PUFA or analog or derivative in combination with DPAn-6 and/or DHA. The amount of the VLC-PUFA or analog or derivative may represent about 30 weight percent or more, about 35 weight percent or more, about 40 weight percent or more, about 45 weight percent or more, about 50 weight percent or more, about 55 weight percent or more, about 60 weight percent or more, about 65 weight percent or more, about 70 weight percent or more, about 75 weight percent or more, or about 80 weight percent or more, or about 85 weight percent or more, or about 90 weight percent or more, or about 95 weight percent or more of the composition of the VLC-PUFA with DPAn-6 and/or DHA. Preferably, the ratio of VLC-PUFA to DHA or DPA (n-6) in the oil, composition, formulation or other product is between about 1:10 to about 10:1, or any ratio between 1:10 and 10:1.

In accordance with the present invention, the VLC-PUFAs and/or derivatives thereof, that are used in oils, supplements, cosmetics, infant formulas, therapeutic and pharmaceutical compositions, and other formulations or products described herein, may be provided in a variety of forms. For example, such forms include, but are not limited to, esters of the VLC-PUFAs; free fatty acids; alcohols; aldehydes; conjugates of the VLC-PUFAs with another bioactive molecule; and combinations thereof. Preferably, the VLC-PUFAs present in these formulations or products are present as ethyl esters. VLC-PUFAs can be blended with long chain fatty acids in amounts and/or ratios that are different from the amounts or ratios that occur in natural sources of the fatty acids, such as by blending, purification, or enrichment. These forms allow flexibility in the formulation of foods with high sensory quality, dietary or nutritional supplements, and pharmaceutical agents.

Any biologically acceptable dosage forms, and combinations thereof, are within the compositions of the invention. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, foods, nutritive foods, functional foods and combinations thereof. Particular dosage formulations include hard or soft gelatin capsules encapsulating an oil having a therapeutically-effective amount of one or more VLC-PUFAs.

The preparations of the above dosage forms are well known to persons of ordinary skill in the art. Preferably, a food (food product) that is enriched with the desired VLC-PUFA and/or VLC-PUFA derivatives thereof, is selected from the group including, but not limited to: baked goods and mixes; chewing gum; cereals; cheese products; nuts and nut-based products; gelatins, pudding, and fillings; frozen dairy products; milk products; dairy product analogs; hard or soft candy; soups and soup mixes; snack foods; processed fruit juice; processed vegetable juice; fats and oils; fish products; plant protein products; poultry products; and meat products.

More particularly, oils containing VLC-PUFAs, and derivatives thereof, will be useful as dietary supplements or therapeutically-effective pharmaceutical compositions in the form of oil-filled capsules or through fortification of foods, beverages or infant formula to enhance the anti-inflammatory benefits of these products. For example, the VLC-PUFAs are provided for delivery in a single dietary supplement. In another application, foods and beverages, including, but not limited to, dairy products and dairy analogs, bakery products and confectionaries, processed meats and meat analogs, grain products and cereals, liquid and powered beverages, including juices and juice drinks, carbonated and processed beverage products or infant formulas may be fortified with VLC-PUFA oils. In another embodiment, VLC-PUFA oils may be microencapsulated prior to fortification of the foods, beverages or formulas to reduce oxidation/degradation of the VLC-PUFAs and to improve organoleptic properties and shelf-life of the fortified food/beverage or infant formula products. In another example, VLC-PUFAs may be formulated into a cream or emulsion for topical applications for preventing or treating skin disorders, including the reduction of inflammation. For example, the VLC-PUFAs may be formulated into sun screens or cosmetics, such as face or hand creams, moisturizers, foundations, eye gels or shaving creams, to reduce skin irritation or redness, allergic reactions or edema.

In another example, more highly enriched or purified forms of the VLC-PUFAs (and in particular VLC-PUFA-rich oils are used in pharmaceutical formulations to prevent or reduce symptoms of conditions or diseases of the eye, and especially the retina, testis or brain. In one embodiment the VLC-PUFAs are administered to a mammal in need of such compounds in the form of a liquid, suspension, emulsion, oil or ointment applied to the eye or eye lids in order to target delivery of these VLC-PUFAs, or analogs or derivatives thereof, to the eye(s) of the mammal.

In one embodiment of the present invention, the formulations containing VLC-PUFAs and/or VLC-PUFA derivatives thereof, including any oils or compositions or formulations containing such LCPUFAs or PUFA derivatives thereof, can be provided with one or more additional components that may be useful in a therapeutic treatment method of the invention. Such additional components include, but are not limited to, any additional anti-inflammatory agent, nutritional supplement (e.g., vitamins, minerals and other nutritional agents, including nutraceutical agents), a therapeutic agent, or a pharmaceutical or a nutritional carrier (e.g., any excipient, diluent, delivery vehicle or carrier compounds and formulations that can be used in conjunction with pharmaceutical (including therapeutic) compositions or nutritional compositions).

One embodiment of the present invention is based on the use of VLC-PUFAs and/or the VLC-PUFA analogs or derivatives, and/or oils or pharmaceutical compositions that have been enriched for VLC-PUFAs to provide cardiovascular, anti-inflammatory, neuroprotective, fertility-enhancing and/or disease prevention effects in humans and other animals. Such effects are useful for enhancing the general health of an individual, as well as in treating or preventing a variety of diseases and conditions in an individual.

Accordingly, one embodiment of the present invention relates to the use of: (1) a VLC-PUFA or VLC-PUFA derivative alone or in combination with other PUFAs and/or other PUFA derivatives; and/or (2) an oil or product produced using such oil, wherein the oil has been enriched in quantity, quality and/or stability of the VLC-LCPUFAs contained therein. The use of these compositions is typically provided by an oil or product using such oil, a nutritional supplement, cosmetic formulation or pharmaceutical composition (medicament or medicine).

Such oils, supplements, compositions and formulations can be used to reduce cardiovascular risk factors in a patient that has or is at risk of developing a cardiovascular disorder or disease, or condition associated with cardiovascular disease. Cardiovascular risk factors may include high blood pressure, elevated serum cholesterol, cigarette smoking, obesity, glucose intolerance, diabetes, left ventricular hypertrophy, low HDL cholesterol, lack of exercise, excessive alcohol intake, and/or high fibrinogen levels.

Such oils, supplements, compositions and formulations can be used for the reduction of inflammation in a patient that has or is at risk of developing inflammation or a disease or condition associated with inflammation. Symptoms of inflammation include both physiological and biological symptoms including, but are not limited to, cytokine production, eicosanoid production, histamine production, bradykinin production, prostaglandin production, leukotriene production, fever, edema or other swelling, pain (e.g., headaches, muscle aches, cramps, joint aches), chills, fatigue/loss of energy, loss of appetite, muscle or joint stiffness, redness of tissues, fluid retention, and accumulation of cellular mediators (e.g., neutrophils, macrophages, lymphocytes, etc.) at the site of inflammation. Diseases associated with inflammation include, but are not limited to, conditions associated with infection by infectious agents (e.g., bacteria, viruses), shock, ischemia, cardiopulmonary diseases, autoimmune diseases, neurodegenerative conditions, and allergic inflammatory conditions, and various other diseases detailed herein.

Such oils, supplements, compositions and formulations can also be used for the reduction of any symptoms related to neurodegeneration or a disease associated with neurodegeneration in a patient that has or is at risk of developing a neurodegenerative condition or disease. Symptoms associated with neurodegeneration include both physiological and biological symptoms including, but not limited to: neurodegeneration, intellectual decline, behavioral disorders, sleep disorders, common medical complications, dementia, psychosis, anxiety, depression, inflammation, pain, and dysphagia.

Such oils, supplements, compositions and formulations can also be used for the reduction of any symptoms related to macular degeneration or a disease associated with macular degeneration in a patient that has or is at risk of developing a degenerative eye condition or disease including Stargardt's disease. Symptoms associated with macular degeneration may include blurred vision, a gradual loss of central vision or a rapid onset of vision loss, central scotomas (shadows or missing areas of vision), distorted vision (i.e. metamorphopsia), trouble discerning colors, slow recovery of visual function after exposure to bright light, and loss in contrast sensitivity.

Such oils, supplements, compositions and formulations can also be used to enhance fertility or treat a disease associated with a fertility disorder, particularly regarding sperm motility or disorders of the testes, in a patient that has or is at risk of developing a fertility disorder or condition or disease, such that increasing the VLC-PUFA content of sperm cells leads to the enhanced production of viable, mature sperm cells. Symptoms associated with disorders of fertility may include decreased or aberrant sperm motility, viability or cold sensitivity within fresh or stored ejaculates.

The compositions and method of the present invention preferably protect the patient from inflammation, neurodegeneration, macular or retinal degeneration, fertility disorders or a condition or disease associated with inflammation. As used herein, the phrase “protected from a disease” (or symptom or condition) refers to reducing the symptoms of the disease; reducing the occurrence of the disease, and/or reducing the severity of the disease. Protecting a patient can refer to the ability of a nutritional or therapeutic composition of the present invention, when administered to the patient, to prevent inflammation from occurring and/or to cure or to alleviate inflammation and/or disease/condition symptoms, signs or causes. As such, to protect a patient from a disease or condition includes both preventing occurrence of the disease or condition (prophylactic treatment) and treating a patient that has a disease or condition or that is experiencing initial symptoms of a disease or condition (therapeutic treatment). The term, “disease” or “condition” refers to any deviation from the normal health of an animal and includes a state when disease symptoms are present, as well as conditions in which a deviation (e.g., infection, gene mutation, genetic defect, etc.) has occurred, but symptoms are not yet manifested.

According to the present invention, the VLC-PUFAs (or analogs or derivatives thereof), compositions comprising such VLC-PUFAs, and methods of the invention, are suitable for use in any individual (subject) that is a member of the Vertebrate class, Mammalia, including, without limitation, primates, livestock and domestic pets (e.g., a companion animal). Most typically, an individual will be a human. According to the present invention, the terms “patient”, “individual” and “subject” can be used interchangeably, and do not necessarily refer to an animal or person who is ill or sick (i.e., the terms can reference a healthy individual or an individual who is not experiencing any symptoms of a disease or condition). In one embodiment, an individual to which an PUFA(s) or composition or formulation or oil of the present invention can be administered includes an individual who is at risk of, diagnosed with, or suspected of having inflammation, neurodegeneration, retinal or macular degeneration or a condition or disease related thereto. Individuals can also be healthy individuals, wherein VLC-PUFAs or compositions of the invention are used to enhance, maintain or stabilize the health of the individual.

The amount of a VLC-PUFA or VLC-PUFA derivative to be administered to an individual can be any amount suitable to provide the desired result of reducing at least one symptom of inflammation, neurodegeneration, or eye disorder or protecting the individual from a condition or disease associated with such disorders. In one embodiment, a VLC-PUFA is administered in a dosage of from about 0.1 mg of the VLC-PUFA per kg body weight of the individual to about 500 mg of the PUFA per kg body weight of the individual, although dosages are not limited to these amounts. A VLC-PUFA derivative or mixture of such derivatives is administered in a dosage of from about 0.2 μg of the PUFA per kg body weight of the individual to about 100 mg of the derivative(s) per kg body weight of the individual, although dosages are not limited to these amounts. The VLC-PUFAs formulated as pharmaceutical compositions are effective over a wide dosage range and are administered in a therapeutically-effective amount. The dosage and manner of administration will be defined by the application of VLC-PUFA and can be determined by routine methods of clinical testing to find the optimum dose. In some embodiments, a therapeutically-effective amount administered to a human comprises about 3 mg/day to about 7.5 g/day of the VLC-PUFA, more particularly about 3 mg/day to about 4 g/day and more particularly 100 mg/day to about 4 g/day. It will be understood, however, that the amount of the pharmaceutical compositions and formulations actually administered will be determined by a physician, in the light of the relevant circumstances, including the condition to be treated, the chosen route of administration, the actual compound administered, the age, weight, and response of the individual patient, the severity of the patient's symptoms, and the like.

Although compositions and formulations of the invention can be administered topically or as an injectable, the oral or ocular routes of administration are also useful. The compositions and formulations used herein may be administered to subjects in the form of nutritional supplements and/or foods (including food products) and/or pharmaceutical formulations.

As discussed above, a variety of additional agents can be included in the compositions when administered or provided to the subject, such as other anti-inflammatory agents, vitamins, minerals, carriers, excipients, and other therapeutic agents. A particular additional agent is aspirin, or another suitable anti-inflammatory agent.

A food product that is enriched with the desired VLC-PUFAs and/or PUFA derivatives thereof may include, but is not limited to: baked goods and mixes; chewing gum; breakfast cereals; cheese products; nuts and nut-based products; gelatins, pudding, and fillings; frozen dairy products; milk products; dairy product analogs; hard or soft candy; soups and soup mixes; snack foods; processed fruit juice; processed vegetable juice; fats and oils; fish products; plant protein products; poultry products; and meat products.

More particularly, oils containing VLC-PUFAs and VLC-PUFA derivatives may be useful as dietary supplements in the form of oil-filled capsules or through fortification of foods, beverages or infant formula.

The pharmaceutical compositions of the present invention contain, as the active ingredient, one or more of the VLC-PUFAs, associated with pharmaceutically acceptable formulations. Preferably, the VLC-PUFAs are present in these pharmaceutical compositions as esters. More preferably, the VLC-PUFAs are present in these pharmaceutical compositions as ethyl esters.

In making the pharmaceutical formulations of this invention, the active ingredient is usually mixed with an excipient, diluted by an excipient or enclosed within a carrier which can be in the form of a capsule, paper or other container. An excipient is usually an inert substance that forms a vehicle for a drug. When the excipient serves as a diluent, it can be a solid, semi-solid, or liquid material, which acts as a vehicle, carrier or medium for the active ingredient. Thus, the compositions can be in the form of solutions, syrups, suspensions, ointments, creams, soft and hard gelatin capsules, suppositories, and injectable solutions, such as parenteral nutrition preparations for injection.

Some examples of suitable excipients include lactose, dextrose, sucrose, sorbitol, mannitol, starches, gum acacia, gum Arabic, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above.

Formulations of the invention suitable for oral administration may be in the form of capsules, pills, tablets, or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a VLC-PUFA of the present invention as an active ingredient.

In solid dosage forms of the invention for oral administration (capsules, tablets, pills, dragees, powders, granules and the like), the active ingredient is mixed with one or more pharmaceutically acceptable carriers, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also include buffering agents. Solid compositions of a similar type may be employed as fillers in soft and hard-filled gelatin capsules using such excipients as lactose or milk sugars, as well as high molecular weight polyethylene glycols and the like.

Liquid dosage forms for oral administration of the VLC-PUFAs of the invention include pharmaceutically-acceptable emulsions, microemulsions, solutions, suspensions, syrups and elixirs. In addition to the active VLC-PUFA ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral formulations may also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the VLC-PUFAs of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of the VLC-PUFAs of this invention include powders, sprays, ointments, pastes, creams, lotions, gels, solutions, patches, drops and inhalants. The active VLC-PUFA ingredient may be mixed under sterile conditions with a pharmaceutically-acceptable carrier, and with any buffers, or propellants which may be required. The ointments, pastes, creams and gels may contain, in addition to an active VLC-PUFA ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane. Transdermal patches have the added advantage of providing controlled delivery of VLC-PUFAs to the skin or body. Such dosage forms can be made by dissolving, dispersing or otherwise incorporating one or more VLC-PUFA compounds in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel. The invention also provides pharmaceutical products suitable for treatment of the eye. Such pharmaceutical products include pharmaceutical compositions, devices and implants (which may be compositions or devices).

Pharmaceutical formulations (compositions) for intraocular injection of a compound or compounds of the invention into the eyeball include solutions, emulsions, suspensions, particles, capsules, microspheres, liposomes, matrices, etc. See, e.g., U.S. Pat. No. 6,060,463, U.S. Patent Application Publication No. 2005/0101582, and PCT application WO 2004/043480, the complete disclosures of which are incorporated herein by reference. For instance, a pharmaceutical formulation for intraocular injection may comprise one or more compounds of the invention in combination with one or more pharmaceutically-acceptable sterile isotonic aqueous or non-aqueous solutions, suspensions or emulsions, which may contain antioxidants, buffers, suspending agents, thickening agents or viscosity-enhancing agents (such as a hyaluronic acid polymer). Examples of suitable aqueous and nonaqueous carriers include water, saline (preferably 0.9%), dextrose in water (preferably 5%), buffers, dimethylsulfoxide, alcohols and polyols (such as glycerol, propylene glycol, polyethylene glycol, and the like). These compositions may also contain adjuvants such as wetting agents and emulsifying agents and dispersing agents. In addition, prolonged absorption of the injectable pharmaceutical form may be brought about by the inclusion of agents which delay absorption such as polymers and gelatin. Injectable depot forms can be made by incorporating the drug into microcapsules or microspheres made of biodegradable polymers such as polylactide-polyglycolide. Examples of other biodegradable polymers include poly(orthoesters), poly(glycolic) acid, poly(lactic) acid, polycaprolactone and poly(anhydrides). Depot injectable formulations are also prepared by entrapping the drug in liposomes (composed of the usual ingredients, such as dipalmitoyl phosphatidylcholine) or microemulsions which are compatible with eye tissue. Depending on the ratio of drug to polymer or lipid, the nature of the particular polymer or lipid components, the type of liposome employed, and whether the microcapsules or microspheres are coated or uncoated, the rate of drug release from microcapsules, microspheres and liposomes can be controlled.

The compounds of the invention can also be administered surgically as an ocular implant. For instance, a reservoir container having a diffusible wall of polyvinyl alcohol or polyvinyl acetate and containing a compound or compounds of the invention can be implanted in or on the sclera. As another example, a compound or compounds of the invention can be incorporated into a polymeric matrix made of a polymer, such as polycaprolactone, poly(glycolic) acid, poly(lactic) acid, poly(anhydride), or a lipid, such as sebacic acid, and may be implanted on the sclera or in the eye. This is usually accomplished with the animal receiving a topical or local anaesthetic and using a small incision made behind the cornea. The matrix is then inserted through the incision and sutured to the sclera.

The compounds of the invention can also be administered topically to the eye, and an embodiment of the invention is a topical pharmaceutical composition suitable for application to the eye. Topical pharmaceutical compositions suitable for application to the eye include solutions, suspensions, dispersions, drops, gels, hydrogels and ointments. See, e.g., U.S. Pat. No. 5,407,926 and PCT applications WO 2004/058289, WO 01/30337 and WO 01/68053, the complete disclosures of all of which are incorporated herein by reference.

Topical formulations suitable for application to the eye comprise one or more compounds of the invention in an aqueous or nonaqueous base. The topical formulations can also include absorption enhancers, permeation enhancers, thickening agents, viscosity enhancers, agents for adjusting and/or maintaining the pH, agents to adjust the osmotic pressure, preservatives, surfactants, buffers, salts (preferably sodium chloride), suspending agents, dispersing agents, solubilizing agents, stabilizers and/or tonicity agents. Topical formulations suitable for application to the eye will preferably comprise an absorption or permeation enhancer to promote absorption or permeation of the compound or compounds of the invention into the eye and/or a thickening agent or viscosity enhancer that is capable of increasing the residence time of a compound or compounds of the invention in the eye. See PCT applications WO 2004/058289, WO 01/30337 and WO 01/68053. Exemplary absorption/permeation enhancers include methysulfonylmethane, alone or in combination with dimethylsulfoxide, carboxylic acids and surfactants. Exemplary thickening agents and viscosity enhancers include dextrans, polyethylene glycols, polyvinylpyrrolidone, polysaccharide gels, Gelrite®, cellulosic polymers (such as hydroxypropyl methylcellulose), carboxyl-containing polymers (such as polymers or copolymers of acrylic acid), polyvinyl alcohol and hyaluronic acid or a salt thereof.

Liquid dosage forms (e.g., solutions, suspensions, dispersions and drops) suitable for treatment of the eye can be prepared, for example, by dissolving, dispersing, suspending, etc. a compound or compounds of the invention in a vehicle, such as, for example, water, saline, aqueous dextrose, glycerol, ethanol and the like, to form a solution, dispersion or suspension. If desired, the pharmaceutical formulation may also contain minor amounts of non-toxic auxillary substances, such as wetting or emulsifying agents, pH buffering agents and the like, for example sodium acetate, sorbitan monolaurate, triethanolamine sodium acetate, triethanolamine oleate, etc.

Aqueous solutions and suspensions suitable for treatment of the eye can include, in addition to a compound or compounds of the invention, preservatives, surfactants, buffers, salts (preferably sodium chloride), tonicity agents and water. If suspensions are used, the particle sizes should be less than 10 μm to minimize eye irritation. If solutions or suspensions are used, the amount delivered to the eye should not exceed 50 μl to avoid excessive spillage from the eye.

Colloidal suspensions suitable for treatment of the eye are generally formed from microparticles (i.e., microspheres, nanospheres, microcapsules or nanocapsules, where microspheres and nanospheres are generally monolithic particles of a polymer matrix in which the formulation is trapped, adsorbed, or otherwise contained, while with microcapsules and nanocapsules the formulation is actually encapsulated). The upper limit for the size of these microparticles is about 5μ, to about 10μ.

Ophthalmic ointments suitable for treatment of the eye include a compound or compounds of the invention in an appropriate base, such as mineral oil, liquid lanolin, white petrolatum, a combination of two or all three of the foregoing, or polyethylene-mineral oil gel. A preservative may optionally be included.

Ophthalmic gels suitable for treatment of the eye include a compound or compounds of the invention suspended in a hydrophilic base, such as Carpobol-940 or a combination of ethanol, water and propylene glycol (e.g., in a ratio of 40:40:20). A gelling agent, such as hydroxylethylcellulose, hydroxypropylcellulose, hydroxypropylmethylcellulose or ammoniated glycyrrhizinate, is used. A preservative and/or a tonicity agent may optionally be included.

Hydrogels suitable for treatment of the eye are formed by incorporation of a swellable, gel-forming polymer, such as those listed above as thickening agents or viscosity enhancers, except that a formulation referred to in the art as a “hydrogel” typically has a higher viscosity than a formulation referred to as a “thickened” solution or suspension. In contrast to such preformed hydrogels, a formulation may also be prepared so to form a hydrogel in situ following application to the eye. Such gels are liquid at room temperature but gel at higher temperatures (and thus are termed “thermoreversible” hydrogels), such as when placed in contact with body fluids. Biocompatible polymers that impart this property include acrylic acid polymers and copolymers, N-isopropylacrylamide derivatives and ABA block copolymers of ethylene oxide and propylene oxide (conventionally referred to as “poloxamers” and available under the Pluronic® tradename from BASF-Wayndotte).

Dispersions may be liposomal, in which case the formulation is enclosed within liposomes (microscopic vesicles composed of alternating aqueous compartments and lipid bilayers).

Eye drops can be formulated with an aqueous or nonaqueous base also comprising one or more dispersing agents, solubilizing agents or suspending agents. Drops can be delivered by means of a simple eye dropper-capped bottle or by means of a plastic bottle adapted to deliver liquid contents dropwise by means of a specially shaped closure.

The compounds of the invention can also be applied topically by means of drug-impregnated solid carrier that is inserted into the eye. Drug release is generally effected by dissolution or bioerosion of the polymer, osmosis, or combinations thereof. Several matrix-type delivery systems can be used. Such systems include hydrophilic soft contact lenses impregnated or soaked with the desired compound of the invention, as well as biodegradable or soluble devices that need not be removed after placement in the eye. These soluble ocular inserts can be composed of any degradable substance that can be tolerated by the eye and that is compatible with the compound of the invention that is to be administered. Such substances include, but are not limited to, poly(vinyl alcohol), polymers and copolymers of polyacrylamide, ethylacrylate and vinylpyrrolidone, as well as cross-linked polypeptides or polysaccharides, such as chitin.

Pharmaceutical formulations include those suitable for ocular administration or for administration to the upper (nasal) or lower respiratory tract. For ocular administration, drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

Pharmaceutical compositions of this invention suitable for parenteral administrations comprise one or more VLC-PUFA compounds in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

Injectable depot forms are made by forming microencapsule matrices of the VLC-PUFAs in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

The pharmaceutical formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

Suitable antioxidant excipients may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

The dosage formulations provided by this invention may contain VLC-PUFAs either alone or in combination with other therapeutically active ingredients, and pharmaceutically acceptable inert excipients. The term ‘pharmaceutically acceptable inert excipients’ includes at least one of diluents, binders, lubricants/glidants, coloring agents and release modifying polymers.

The dosage form may include one or more diluents such as lactose, sugar, cornstarch, modified cornstarch, mannitol, sorbitol, and/or cellulose derivatives such as wood cellulose and microcrystalline cellulose, typically in an amount within the range of from about 20% to about 80%, by weight.

The dosage form may include one or more binders in an amount of up to about 60% w/w. Examples of suitable binders include methyl cellulose, hydroxypropyl cellulose, hydroxypropylmethyl cellulose, polyvinyl pyrrolidone, eudragits, ethyl cellulose, gelatin, gum arabic, polyvinyl alcohol, pullulan, carbomer, pregelatinized starch, agar, tragacanth, sodium alginate, microcrystalline cellulose and the like.

Examples of suitable disintegrants include sodium starch glycolate, croscarmellose sodium, crospovidone, low substituted hydroxypropyl cellulose, and the like. The concentration may vary from 0.1% to 15%, by weight, of the dosage form.

Examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like. The concentration may vary from 0.1% to 15%, by weight, of the dosage form.

Coloring agents may be selected from the FDA approved colorants including, for example, Iron oxide, Lake of Tartrazine, Allura red, Lake of Quinoline yellow, Lake of Erythrosine.

Another embodiment of the invention relates to the use of any of the VLC-PUFA or compositions or formulations described herein in the preparation of a medicament for the treatment of inflammatory conditions, neurodegenerative disorders and macular or retinal disorders.

The invention now being generally described will be more readily understood by reference to the following example, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Synthesis Procedures

### Preparation of DHA Alcohol:

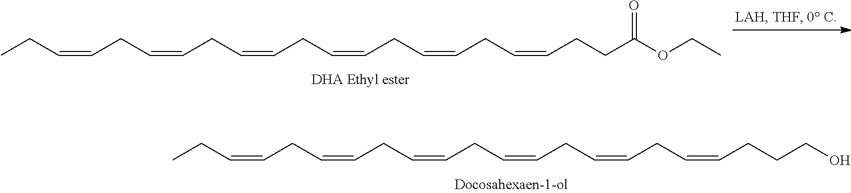

An oven-dried 1 L round bottomed flask was charged with Lithium aluminum hydride (LAH) (4.17 g, 110 mmol) in anhydrous THF (60 mL). The flask was then cooled to 0° C. To this was added drop wise, a solution of DHA ethyl ester (35.6 g, 100 mmol) in THF (40 mL) via addition funnel. After the addition was complete the reaction was allowed to stir for 2 h at 0° C. The reaction was monitored by TLC. After the reaction was complete, it was quenched at 0° C. by slow drop wise addition of saturated aqueous solution of sodium sulfate. The mixture was then allowed to stir for 10-15 min and then filtered through Buchner funnel. The residue was washed with THF. The filtrate and washings were combined and concentrated under reduced pressure to obtain the product. The identity of the alcohol was confirmed by IR and GC-MS

The alcohol was used without further purification.

### Preparation of DHA Bromide:

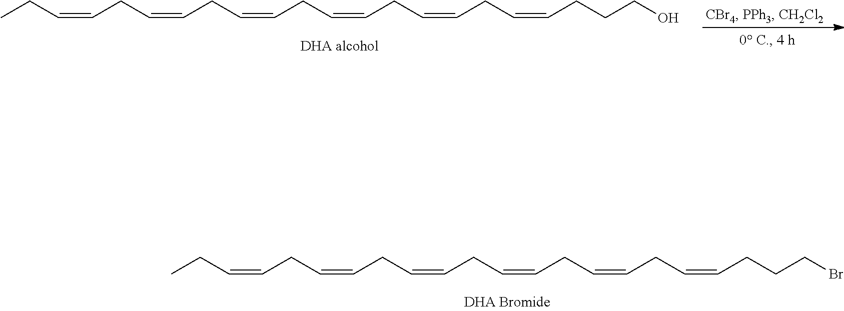 [SMILES: CC/C=C\C/C=C\C/C=C\C/C=C\C/C=C\C/C=C\CCCO ; CC/C=C\C/C=C\C/C=C\C/C=C\C/C=C\C/C=C\CCCBr]

An oven-dried three-neck round bottomed flask was charged with DHA alcohol (5.0 g, 15.9 mmol) and carbon tetrabromide (5.27 g, 15.9 mmol) in anhydrous CH₂Cl₂ (20 mL). The flask was cooled to 0° C. (ice-water bath). To this mixture was added triphenylphosphine (4.17 g, 15.9 mmol) in 4 portions with an interval of 15 min between in each portion. The resulting mixture was allowed to stir for 4 h at 0° C. The reaction mixture was concentrated under reduced pressure. Hexane was added and the mixture was cooled and filtered to remove triphenylphosphine oxide. This process was repeated 2 more times to remove the majority of the byproduct. The crude product was then purified by flash column chromatography using hexanes to 8% ether in hexanes to obtain pure DHA bromide.

### Preparation of 6-tetrahydropyranyloxyhexyl magnesium bromide

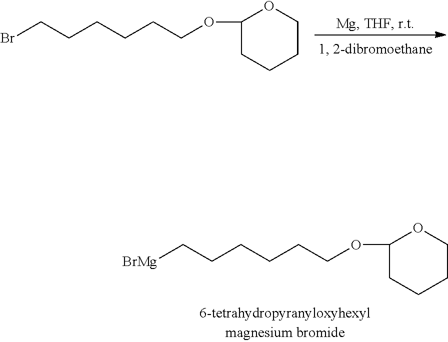

An oven-dried 25 mL round bottomed flask was charged with magnesium turnings (725 mg, 30.2 mmol) in anhydrous THF (10 mL). To this was added a solution of 1-Bromo-6-tetrahydropyranyloxyhexane 2.0 g, 7.55 mmol) in anhydrous THF (5 mL) via a syringe at r.t. After 0.5 mL solution was added, the reaction flask was gently warmed with heat gun. 1,2-dibromoethane (0.2 mL, 2.31 mmol). The remaining solution of the bromo compound was then added and the mixture was allowed to stir for 18 h to obtain 6-tetrahydropynyloxyhexyl magnesium bromide.

### Coupling Reaction of DHA bromide with 6-tetrahydropyranyloxyhexyl magnesium bromide catalyzed by Li₂CuCl₄

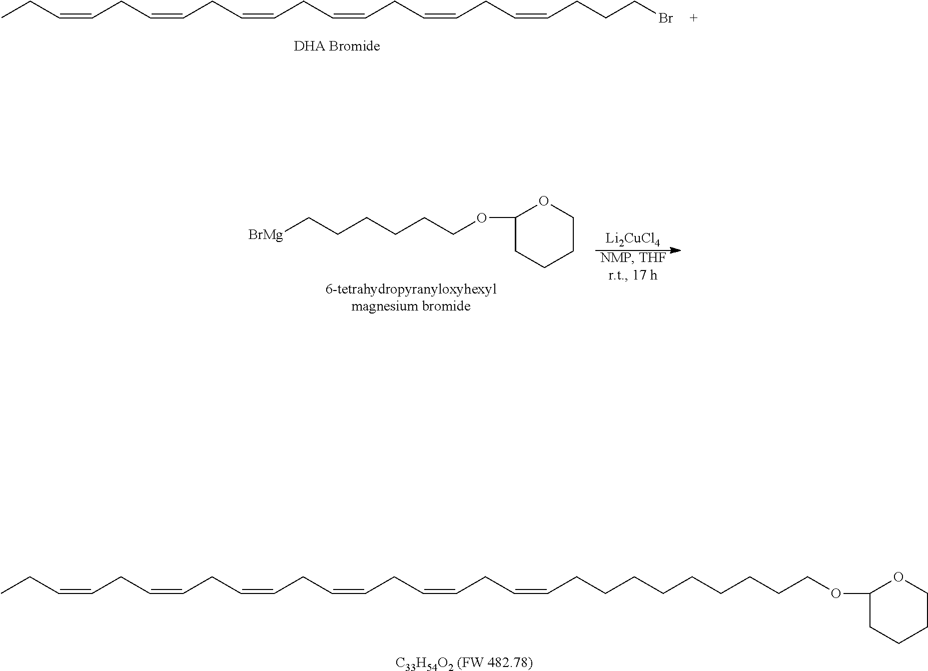

A 25-mL round bottomed flask was charged with DHA bromide (200 mg, 0.53 mmol). The flask was evacuated and filled with N₂ Anhydrous THF (2 mL) was added via syringe followed by a solution of Li₂CuCl₄ (0.2 mL of 0.1 M solution in THF, 0.02 mmol) and N-methyl-2-pyrrolidone (0.2 mL, 2.12 mmol). To this mixture was added a solution of 6-tetrahydropyranyloxyhexyl magnesium bromide (1.84 mL of 0.5 M solution in THF, 0.91 mmol) via syringe at room temperature. The resulting reaction mixture was allowed to stir at room temperature for 17 h. The reaction was then quenched by addition of sat. solution of NH₄Cl (10 mL). It was extracted with CH₂Cl₂ (25 mL×3). The combined organic phase was dried (Na₂SO₄), filtered and concentrated under reduced pressure to obtain the crude product. The product was purified by flash column chromatography using a gradient of hexanes to 5% of ethyl acetate in hexanes to obtain 142 mg of the desired product. LC-MS analysis of the reaction mixture showed a peak at 505 corresponding to the molecular ion [M+Na].

### Deprotection of THP Protected C28:6 Alcohol:

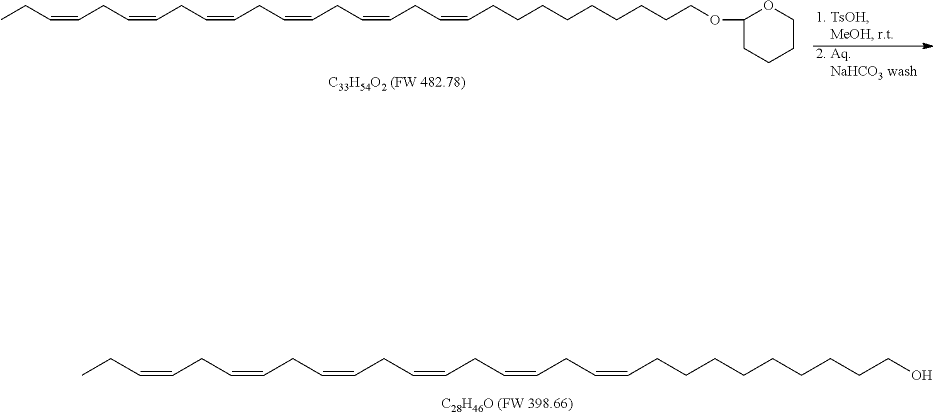

THP protected C28:6 alcohol (730 mg, 1.51 mmol) was dissolved in methanol. p-toluenesulphonic acid monohydrate was added and the mixture was allowed to stir for 6 h at room temperature, NaHCO₃ was added and stirring was continued for 1 h. The mixture was concentrated under reduced pressure, hexane and washed with water. The organic phase was dried (Na₂SO₄), filtered and concentrated under reduced pressure to obtain the crude product. It was purified by flash column chromatography using a gradient of hexanes to 5% ethyl acetate in hexanes to obtain 390 mg of the desired product. LC-MS analysis of the reaction mixture showed a peak at 399 corresponding to the molecular ion [M+H].

### Synthesis of C28:6 VLC PUFA

Oxidation of the C28:6 Alcohol with H₅IO₆/CrO₃:

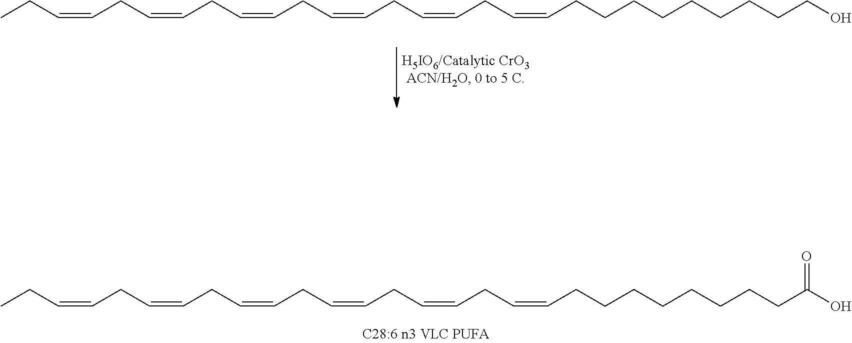 [SMILES: CC/C=C\C/C=C\C/C=C\C/C=C\C/C=C\C/C=C\CCCCCCCCCO ; CC/C=C\C/C=C\C/C=C\C/C=C\C/C=C\C/C=C\CCCCCCCCC(=O)O]

A stock solution of H₅IO₆/CrO₃ was prepared by dissolving H5IO6 (5.7 g, 25 mmol) and CrO₃ (12 mg, 1.2 mol %) in wet MeCN (0.75 v % water) to a volume of 55.7 mL (complete dissolution in about 1.5 hours). The H₅IO₆/CrO₃ solution (1.2 mL) was then added to a solution of the C28:6 alcohol (42 mg, 0.15 mmol) in wet acetonitrile (1.0 mL, 0.75 v % water) slowly while maintaining the reaction temperature at 0-5° C. The mixture was allowed to stir at 0° C. for 0.5 h and the completion of the reaction was confirmed by TLC assay. The reaction was quenched by adding 1 mL aqueous solution of Na₂HPO₄ (0.60 g in 10 mL H₂O). The reaction mixture was extracted twice with 5 mL using ethyl acetate. The ethyl acetate layer was then washed with sodium bisulfate solution and dried over anhydrous MgSO₄ and then concentrated to give 39 mg desired carboxylic acid C28:6 n3 VLC PUFA.

### Synthesis of C34:6 VLC PUFA

### Bromination of C28:6 Alcohol:

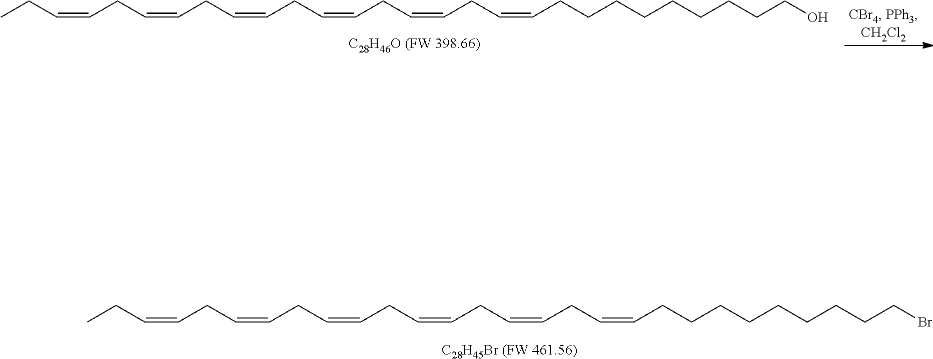 [SMILES: CC/C=C\C/C=C\C/C=C\C/C=C\C/C=C\C/C=C\CCCCCCCCCO ; CC/C=C\C/C=C\C/C=C\C/C=C\C/C=C\C/C=C\CCCCCCCCCBr]

An oven-dried 25 mL round bottomed flask was charged with alcohol (465 mg, 1.17 mmol) in anhydrous CH₂Cl₂ (10 mL) and carbon tetrabromide (583 mg, 1.76 mmol). This mixture was cooled to 0° C. (ice-water bath). To this mixture was added triphenylphosphine (461 mg, 1.76 mmol) in one portion. The reaction mixture was allowed to stir at 0 to 10° C. for 18 h. The reaction mixture was then concentrated under reduced pressure and purified by flash column chromatography to obtain 265 mg of desired product.

### Coupling Reaction of C28:6 bromide with 6-tetrahydropyranyloxyhexyl magnesium bromide catalyzed by Li₂CuCl₄ to Protected C34:6 alcohol

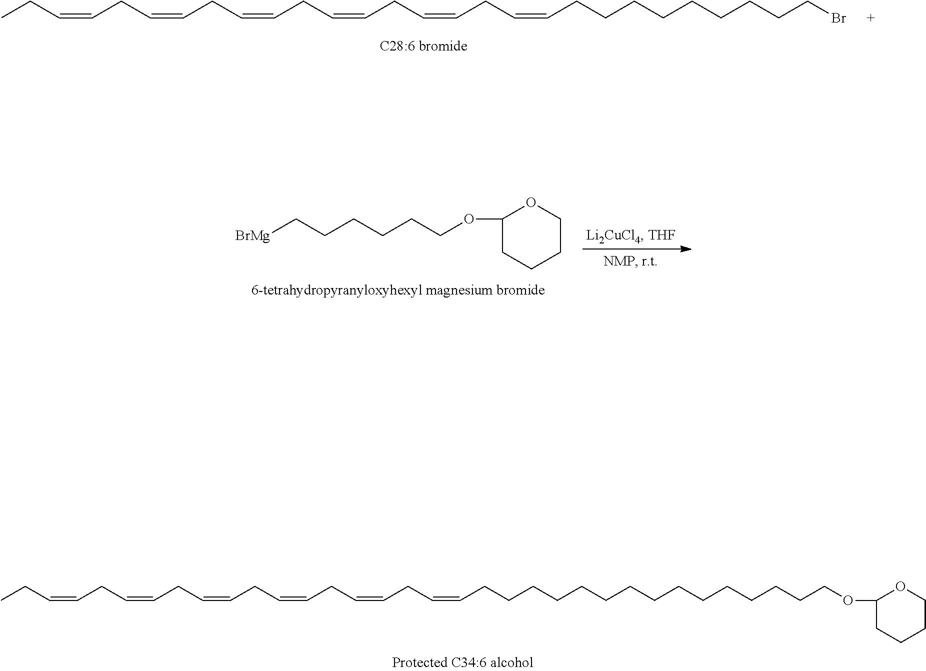

A 25-mL round bottomed flask was charged with C28:6 bromide (210 mg, 0.46 mmol). The flask was evacuated and filled with N₂ Anhydrous THF (2 mL) was added via syringe followed by a solution of Li₂CuCl₄ (0.14 mL of 0.1 M solution in THF, 0.014 mmol) and N-methyl-2-pyrrolidone (0.18 mL, 1.84 mmol). To this mixture was added a solution of 6-tetrahydropyranyloxyhexyl magnesium bromide (1.56 mL of 0.5 M solution in THF, 0.78 mmol) via syringe at room temperature. The resulting reaction mixture was allowed to stir at room temperature for 17 h. The reaction was then quenched by addition of saturated solution of NH₄Cl (10 mL). It was extracted with CH₂Cl₂ (25 mL×3). The combined organic phase was dried (Na₂SO₄), filtered and concentrated under reduced pressure to obtain the desired product.

C34:6 VLC PUFA from Protected C34:6 Alcohol

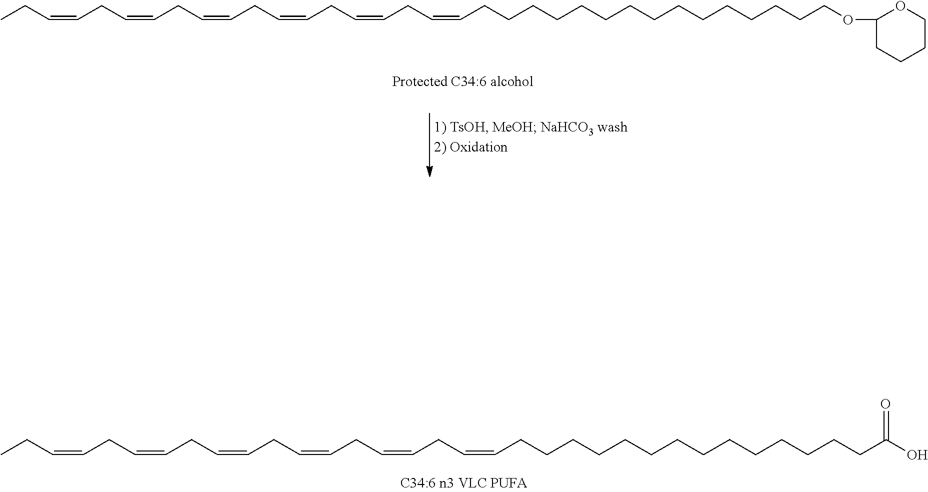 [SMILES: CC/C=C\C/C=C\C/C=C\C/C=C\C/C=C\C/C=C\CCCCCCCCCCCCCCCOC1CCCCO1 ; CC/C=C\C/C=C\C/C=C\C/C=C\C/C=C\C/C=C\CCCCCCCCCCCCCCC(=O)O]

The C34:6 VLC PUFA will be synthesized as shown above by following the process for the synthesis of C28:6 VLC PUFA described earlier.

Alternatively C34:6 VLC PUFA could Also be Obtained as Shown Below.

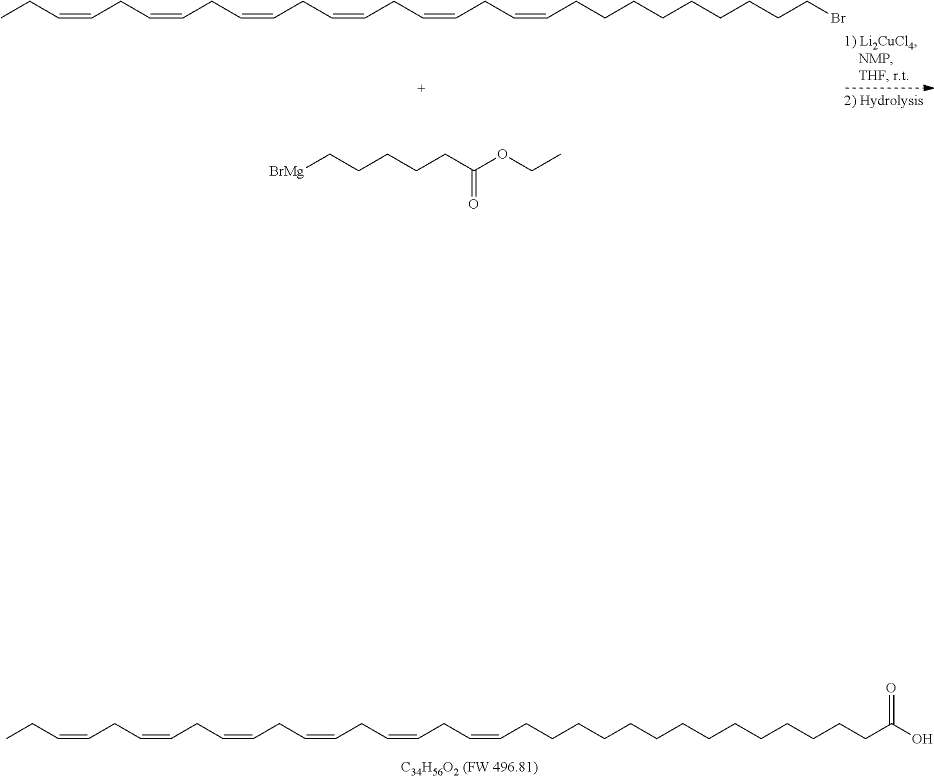

### Preparation of DHA Aldehyde:

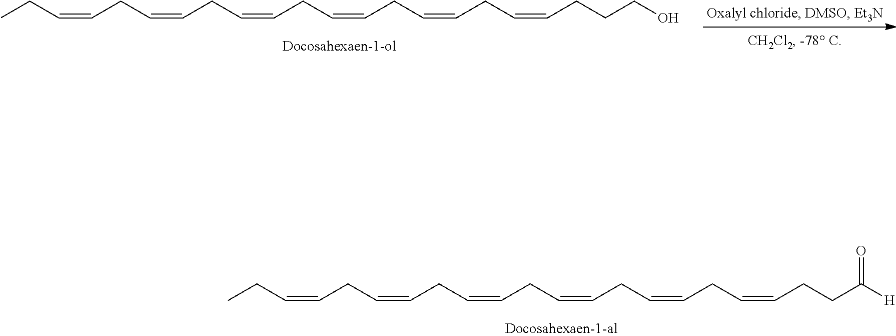 [SMILES: CC/C=C\C/C=C\C/C=C\C/C=C\C/C=C\C/C=C\CCCO ; CC/C=C\C/C=C\C/C=C\C/C=C\C/C=C\C/C=C\CCC=O]

An oven-dried 100 mL round bottomed flask was charged with oxalyl chloride (1.65 mL, 19.1 mmol) in anhydrous CH₂Cl₂ (5 mL) and cooled to −78° C. Dimethyl sulfoxide (2.72 mL, 38.2 mmol) was added to this flask, drop wise, and after 10 minutes, a solution of DHA alcohol (3.0 g, 9.6 mmol) in anhydrous CH₂Cl₂ (5 mL) was added drop wise with a syringe. The resulting mixture was continuously stirred at −78° C. for 50 minutes. Et₃N (5.33 mL, 38.2 mmol) was added drop wise and the mixture was allowed to stir for another 25 minutes. Water was added and the reaction mixture was warmed to room temperature and extracted with CH₂Cl₂ (50 mL×3). The combined organic layer was dried (Na₂SO₄), filtered and concentrated under reduced pressure to obtain the product. The aldehyde was purified by silica-gel column chromatography using hexane-ethyl acetate eluents.

### Coupling Reaction: Procedure-1

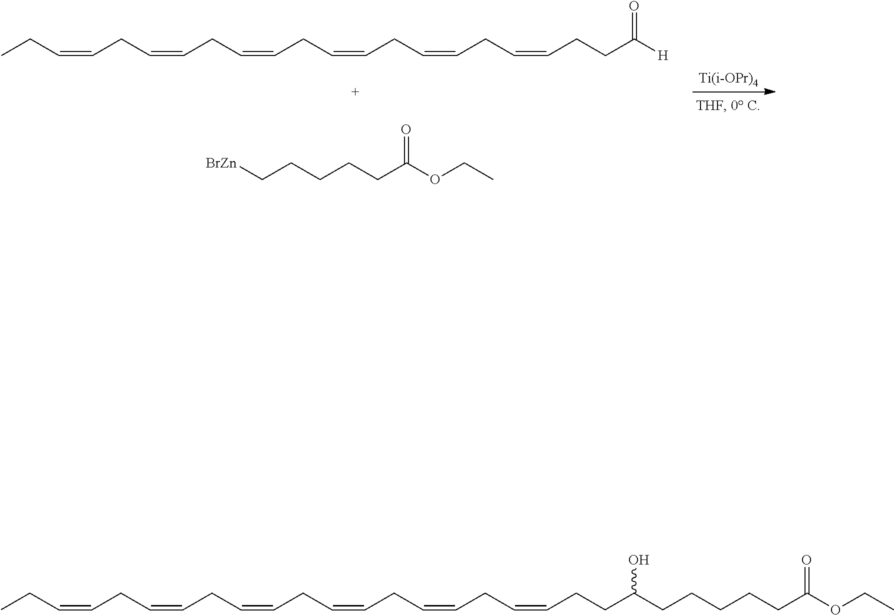

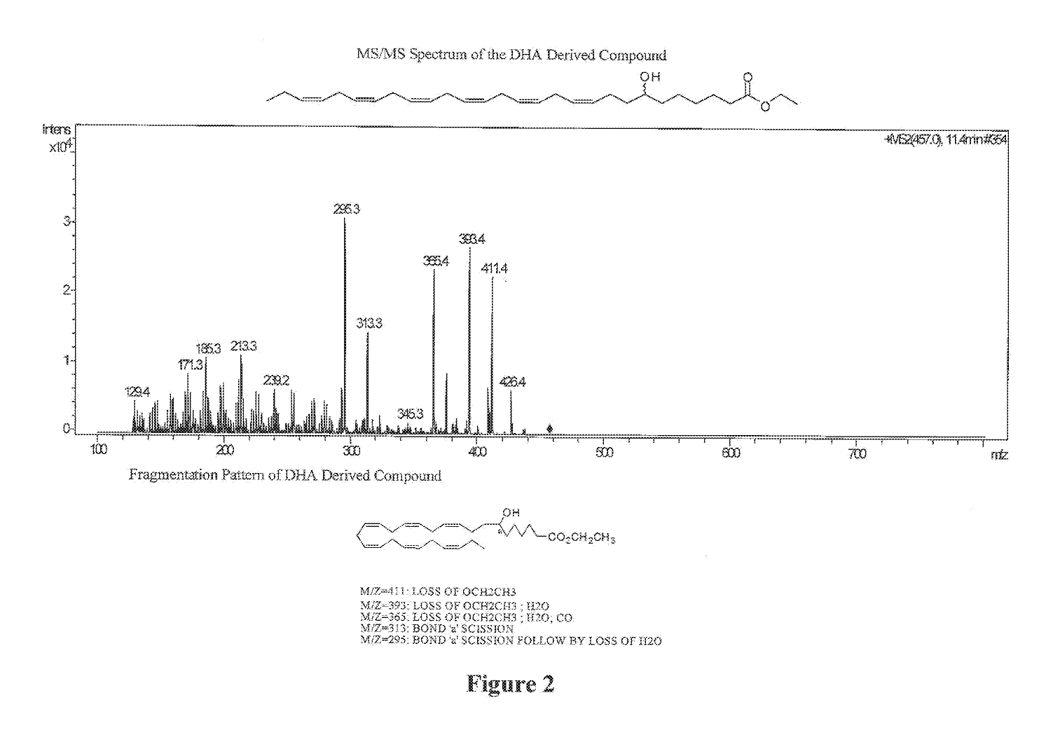

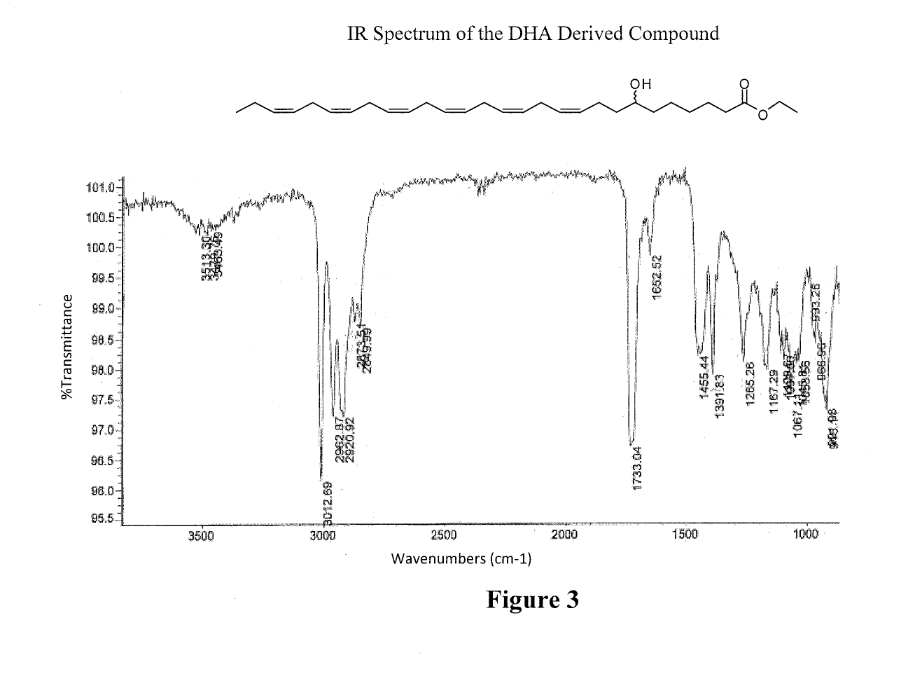

An oven-dried 10 mL round bottomed flask was charged with DHA aldehyde (100 mg, 0.321 mmol) and Ti (i-OPr)₄ (29 μL, 0.10 mmol) in anhydrous THF (1 mL). It was cooled to 0° C. (ice-water bath). To this was added drop wise 6-Ethoxy-6-oxohexylzinc bromide (0.84 mL of 0.5 M solution in THF, 0.42 mmol). The resulting reaction mixture was allowed to stir at 0° C. for 9 h. The reaction was monitored by TLC. After 9 h at 0° C., the reaction was quenched by addition of aqueous HCl. The aqueous layer was extracted with CH₂Cl₂ (20 mL×3). The combined organic layer was dried (Na₂SO₄), filtered and concentrated under reduced pressure to obtain the product. The product was isolated by preparative TLC using hexane-ethyl acetate solvent system. The structure was established by IR, LC-MS and LC-MS/MS, as shown in FIGS. 2 and 3.

### Coupling Reaction: Procedure-2

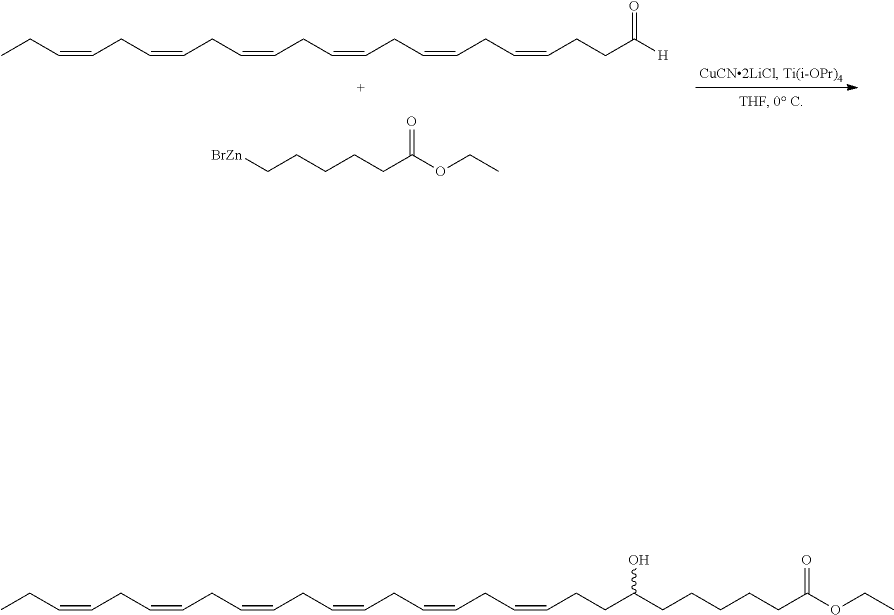

An oven-dried 10 mL round bottomed flask was charged with CuCN (43 mg, 0.48 mmol) and LiCl (43 mg, 1.01 mmol) in anhydrous THF (1 mL). This mixture was allowed to stir at room temperature for 10-15 min. It was then cooled to −20° C. At this temperature was added drop wise 6-Ethoxy-6-oxohexylzinc bromide (0.97 mL of 0.5 M solution in THF, 0.48 mmol). After stirring for 10-15 min. at −20° C., it was allowed to stir at 0° C. for another 10 min. Then at 0° C., a solution of DHA aldehyde (100 mg, 0.321 mmol) in anhydrous THF (1 mL) was added drop wise followed by Ti (i-OPr)₄ (29 μL, 0.10 mmol). The resulting reaction mixture was allowed to stir at 0° C. for 7 h. The reaction was monitored by TLC. After 7 h at 0° C., the reaction was quenched by addition of aqueous HCl. The aqueous layer was extracted with CH₂Cl₂ (20 mL×3). The combined organic layer was dried (Na₂SO₄), filtered and concentrated under reduced pressure to obtain the product.

### Reduction of Secondary Alcohol and Hydrolysis:

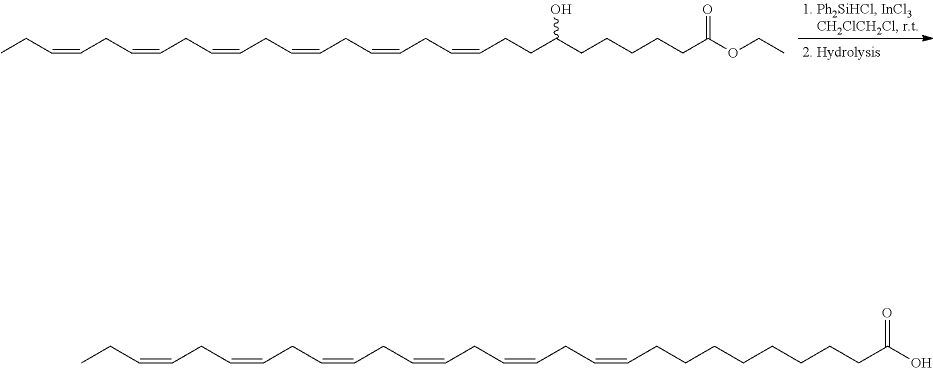

### Palladium Catalyzed Coupling Reaction:

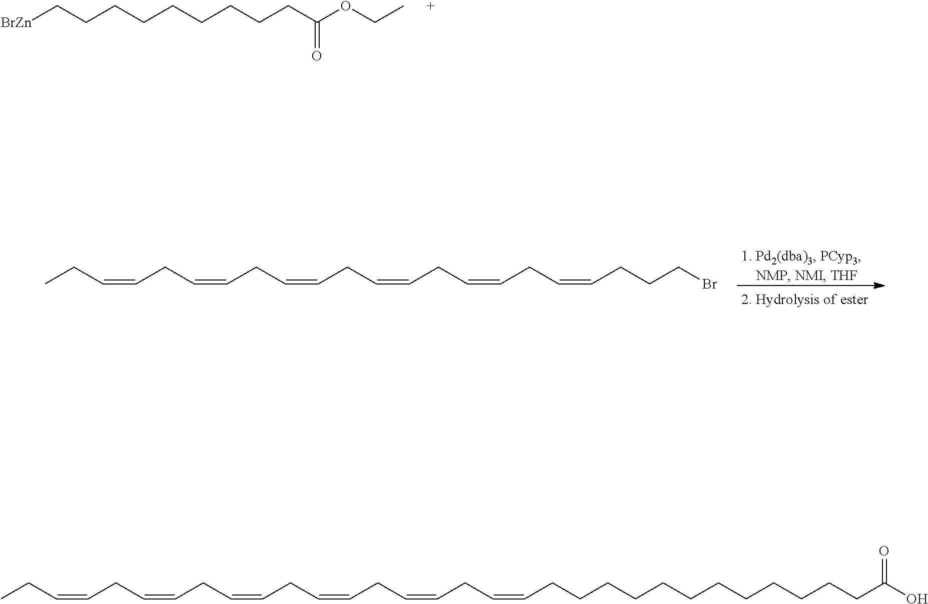

### Example 2

### Synthetic Procedures

DHA bromide was prepared as described in Example 1. 6-tetrahydropyranyloxyhexyl magnesium chloride was prepared as follows:

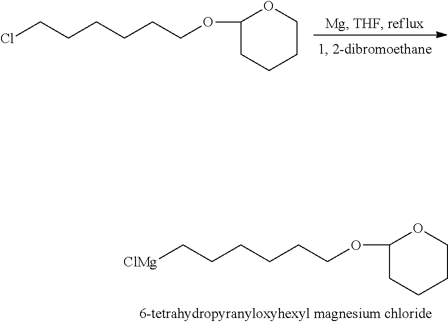

An oven dried 3 necked 500 mL round bottomed flask, attached with a thermometer, addition funnel and a water condenser was charged with magnesium turnings (11.5 g, 480 mmol). The flask was evacuated and filled with N₂ Anhydrous THF (185 mL) was added. The addition was filled with a solution of 1-chloro-6-tetrahydropyranyloxyhexane (26.5 g, 120 mmol) in anhydrous THF (55 mL). 20 mL of this solution was added drop wise to the reaction flask containing Mg turnings in THF at reflux. 1,2-dibromoethane (1.2 mL, 13.2 mmol) was added via syringe. After 5 min, the remaining solution from the addition funnel was added dropwise over a period of 30 min. The reaction mixture was allowed to reflux for 90 min to obtain 6-tetrahydropynyloxyhexyl magnesium chloride.

### Coupling Reaction of DHA bromide with 6-tetrahydropyranyloxyhexyl magnesium chloride catalyzed by Li₂CuCl₄

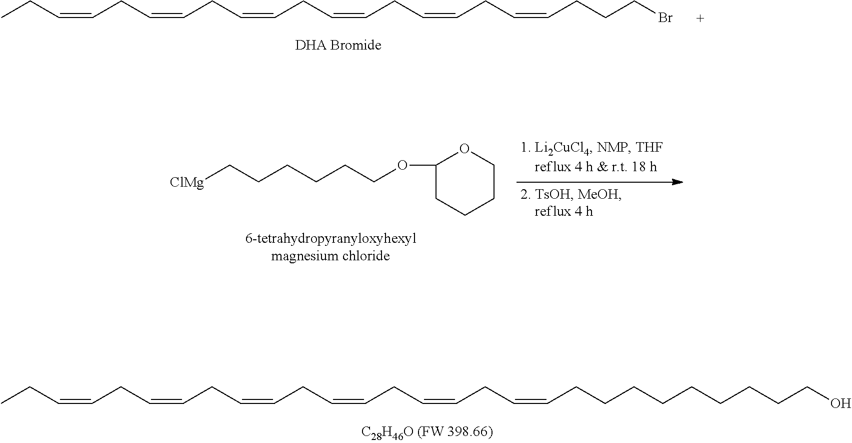

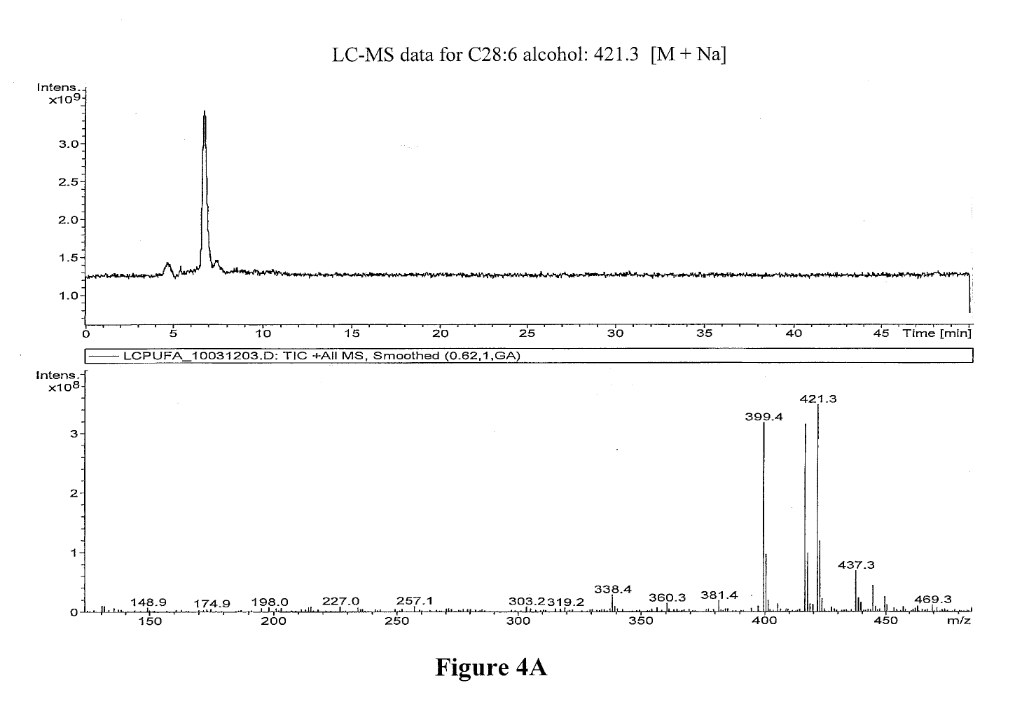

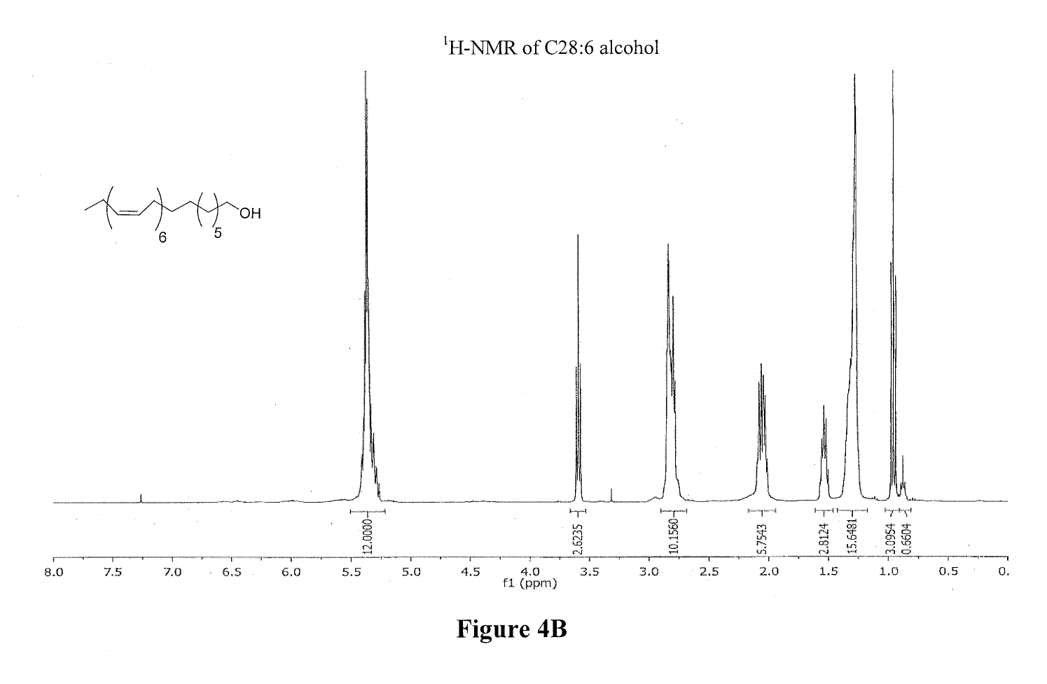

An oven dried 3 necked 1 L round bottomed flask, attached with a thermometer, addition funnel and a water condenser was charged with was charged with DHA bromide (22.6 g, 60 mmol). The flask was evacuated and filled with N₂ Anhydrous THF (88 mL) was added and the mixture was heated to 55 to 60° C. A solution of Li₂CuCl₄ (18 mL of 0.1 M solution in THF, 1.8 mmol) was added dropwise via a syringe. After 10 min N-methyl-2-pyrrolidone (23.2 mL, 240 mmol) was added dropwise via a syringe. This mixture was allowed to stir at 60° C. for 10 min and then was added a solution of 6-tetrahydropyranyloxyhexyl magnesium chloride (240 mL of 0.5 M solution in THF, 120 mmol) via addition funnel. The resulting reaction mixture was allowed to reflux for 4 h and then at room temperature for 18 h. The reaction was then quenched by addition of a saturated solution of NH₄Cl (150 mL). Most of the THF from the reaction mixture was then removed by rotary evaporator. The aqueous layer was extracted with CH₂Cl₂ (150 mL×3). The combined organic phase was dried (Na₂SO₄), filtered and concentrated under reduced pressure to obtain the crude product. This crude THP protected C28:6 alcohol was then transferred to an oven dried 3 necked 500 mL round bottomed flask. Methanol (150 mL) was then added followed by p-toluenesulphonic acid monohydrate (8 g, 42.1 mmol). The mixture was allowed to reflux for 4 h. The reaction mixture was then concentrated under reduced pressure and CH₂Cl₂ (200 mL) was added. It was then washed with saturated solution of NaHCO₃ (150 mL×3) and brine (100 mL). The organic layer was dried (Na₂SO₄), filtered and concentrated under reduced pressure to obtain the crude product. It was purified by flash column chromatography using a gradient of hexanes to 10% ethyl acetate in hexanes to obtain 17 g of the desired product (71% yield). The NMR and LC-MS spectra for the product are shown in FIG. 4.

Oxidation of the C28:6 Alcohol:

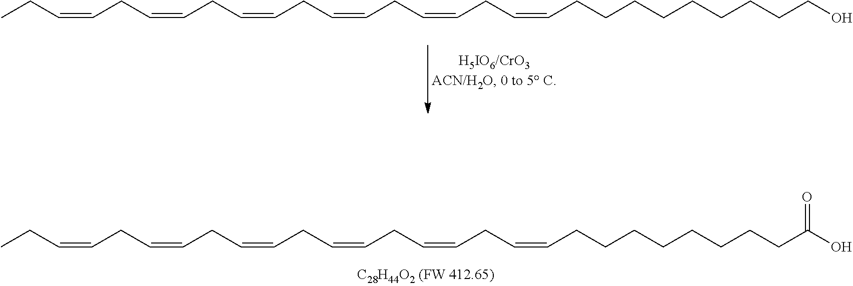 [SMILES: CC/C=C\C/C=C\C/C=C\C/C=C\C/C=C\C/C=C\CCCCCCCCCO ; CC/C=C\C/C=C\C/C=C\C/C=C\C/C=C\C/C=C\CCCCCCCCC(=O)O]

Preparation of H₅IO₆/CrO₃ Stock Solution:

H₅IO₆ (11.4 g) and CrO₃ (23 mg) was dissolved in wet ACN (0.75 v % H₂O).

25 mL 3 necked round bottomed flask attached with a thermometer and an addition funnel was charged with C28:5 alcohol (500 mg, 1.25 mmol). 0.75 v % wet THF (10 mL) was added via a syringe. The mixture was cooled to 0° C. (ice-water bath). To this mixture was added H₅IO₆/CrO₃ stock solution (35 mL) dropwise at such a rate that the temperature does not rise above 5° C. The resulting reaction mixture was allowed to stir at 0 to 5° C. for 3 h. The reaction was then quenched by the addition of an aqueous solution of Na₂HPO₄ (30 mL of 0.6 g/10 mL of H₂O). Ethyl acetate (40 mL) was added. The organic layer was washed with brine (50 mL×2) and aq. solution of sodium bisulfite (60 mL×2 of 2.2 g/50 mL of H₂O). The organic layer was dried (Na₂SO₄), filtered and concentrated under reduced pressure to obtain the crude product. It was purified by flash column chromatography using a gradient of hexanes to 10% ethyl acetate in hexanes to obtain 260 mg of the desired product.

Bromination of C28:6 Alcohol:

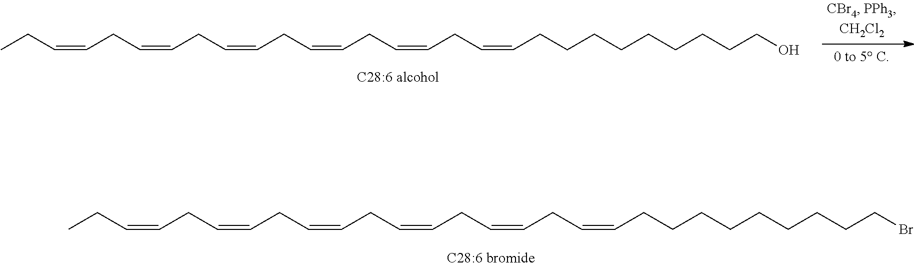 [SMILES: CC/C=C\C/C=C\C/C=C\C/C=C\C/C=C\C/C=C\CCCCCCCCCO ; CC/C=C\C/C=C\C/C=C\C/C=C\C/C=C\C/C=C\CCCCCCCCCBr]

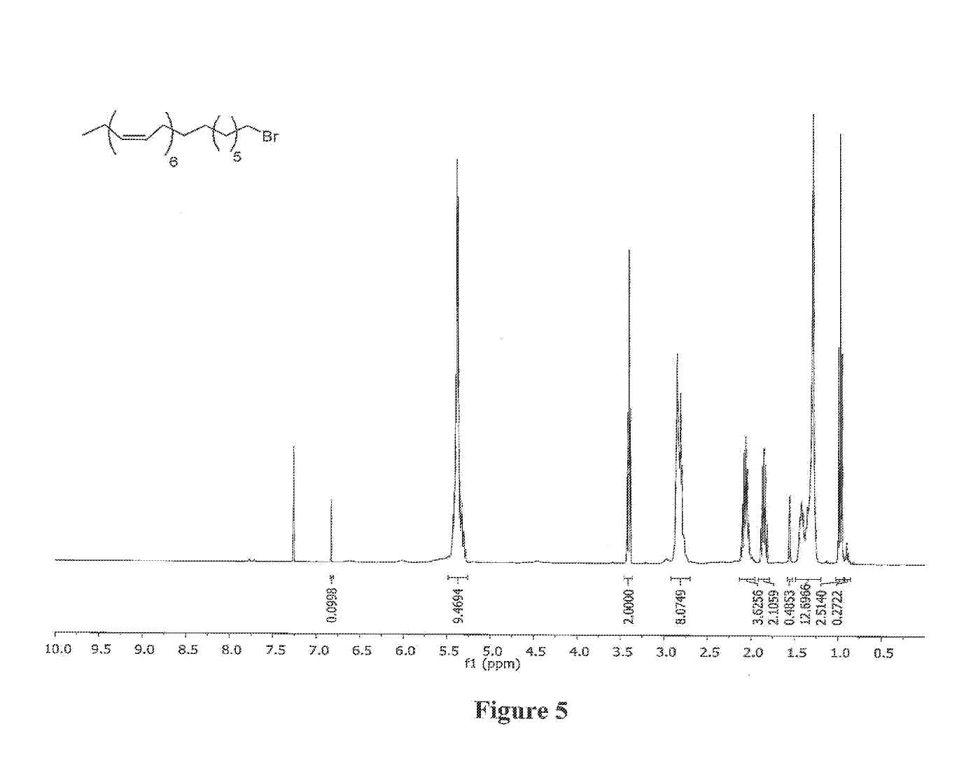

An oven dried 3 necked 250 mL round bottomed flask attached with a thermometer was charged with C28:6 alcohol (18.2 g, 45.8 mmol) Anhydrous CH₂Cl₂ (50 mL) was added via a syringe and the mixture was cooled to 0° C. (ice-water bath). Carbon tetrabromide (18.3 g, 55 mmol) was added in one portion. To this mixture was added triphenylphosphine (14.4 g, 55 mmol) in 4 portions with the interval of 15 min between each portion. The reaction mixture was allowed to stir at 0 to 5° C. for 2 h. The reaction mixture was then concentrated under reduced pressure. Hexane (100 mL) was added and the mixture was filtered to remove triphenylphosphine oxide byproduct. This step was repeated to remove most of the byproduct. The crude product obtained was purified by flash column chromatography using a gradient of hexanes to 3% ethyl acetate in hexanes to obtain 20.2 g of the desired product (96% yield). The NMR spectrum is shown in FIG. 5.

Coupling Reaction of C28:6 bromide with 6-tetrahydropyranyloxyhexyl magnesium chloride catalyzed by Li₂CuCl₄:

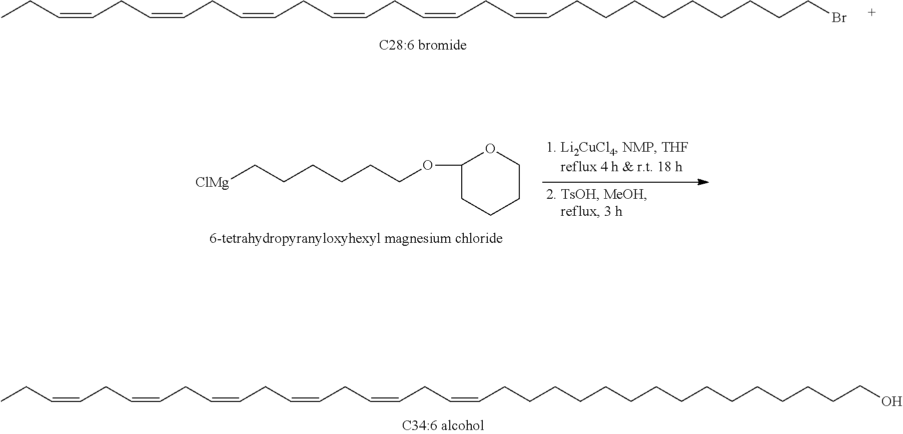

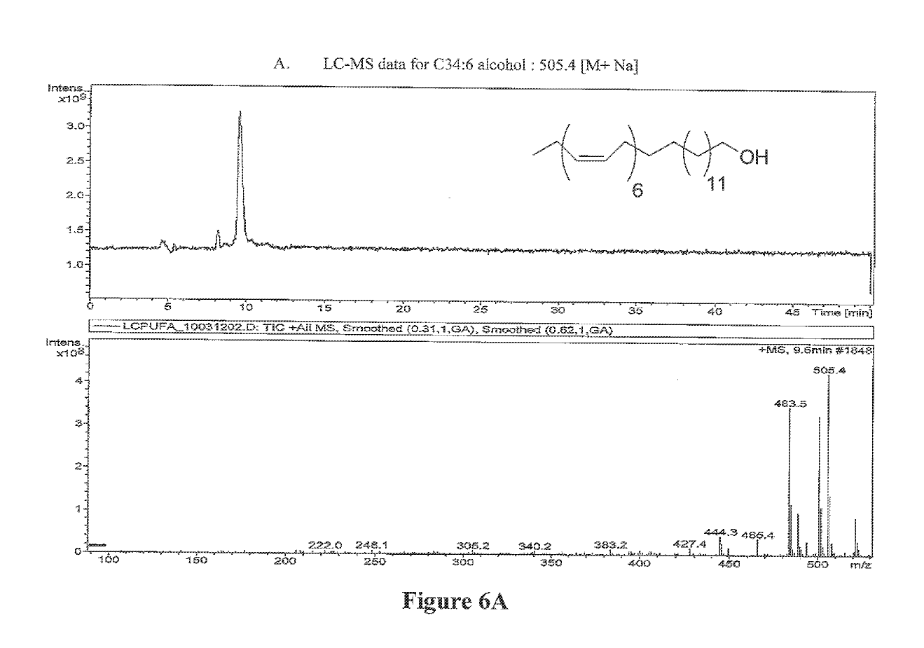

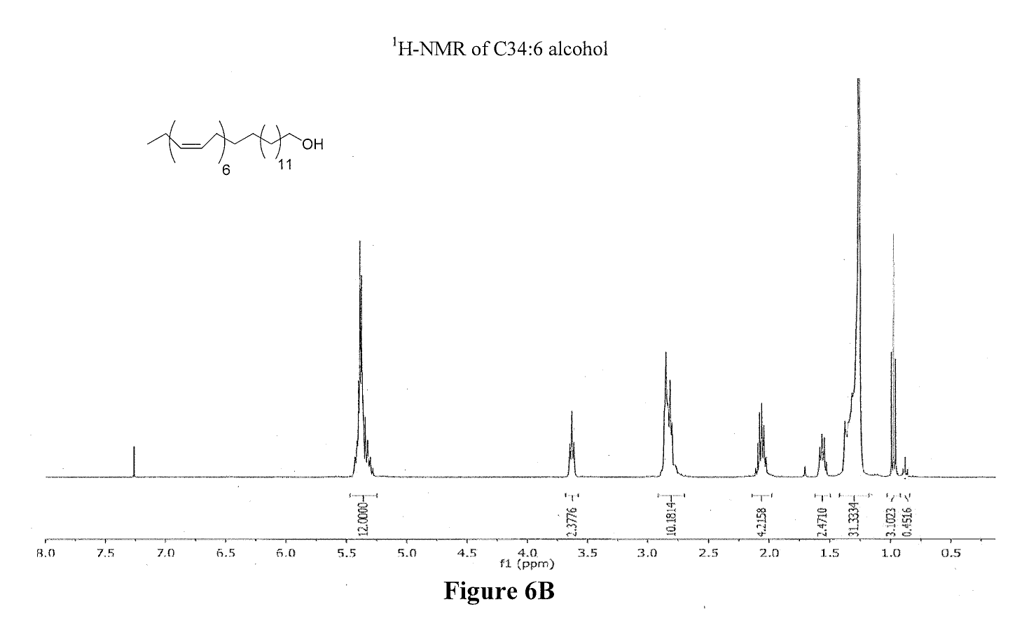

An oven dried 3-necked 250 mL round bottomed flask attached with a thermometer, a water condenser and an addition funnel was charged with C28:6 bromide (7.50 g, 16.3 mmol). The flask was evacuated and filled with N₂ Anhydrous THF (30 mL) was added via a syringe and the mixture was heated to 60° C. A solution of Li₂CuCl₄ (4.9 mL of 0.1 M solution in THF, 0.49 mmol) was added dropwise via a syringe. After 10 min N-methyl-2-pyrrolidone (6.3 mL, 65.2 mmol) was added and the mixture was allowed to stir at reflux temperature. After 10 min was added a solution of 6-tetrahydropyranyloxyhexyl magnesium chloride (82 mL of 0.5 M solution in THF, 41 mmol) via the addition funnel. The resulting reaction mixture was allowed to reflux for 4 h and then stir at room temperature for 18 h. The reaction was then quenched by addition of saturated solution of NH₄Cl (100 mL). It was extracted with CH₂Cl₂ (100 mL×3). The combined organic layer was dried (Na₂SO₄), filtered and concentrated under reduced pressure to obtain the crude product. This crude THP protected C34:6 alcohol was then transferred to an oven dried 3 necked 250 mL round bottomed flask. Methanol (60 mL) was then added followed by p-toluenesulphonic acid monohydrate (3 g, 15.8 mmol). The mixture was allowed to reflux for 3 h. The reaction mixture was then concentrated under reduced pressure and CH₂Cl₂ (200 mL) was added. It was then washed with saturated solution of NaHCO₃ (100 mL×3) and brine (100 mL). The organic layer was dried (Na₂SO₄), filtered and concentrated under reduced pressure to obtain the crude product. It was purified by flash column chromatography using a gradient of hexanes to 10% ethyl acetate in hexanes to obtain 4.7 g of the desired product (60% yield). The LC-MS and NMR data for the C34:6 alcohol product are shown in FIG. 6.

Oxidation of C34:6 alcohol:

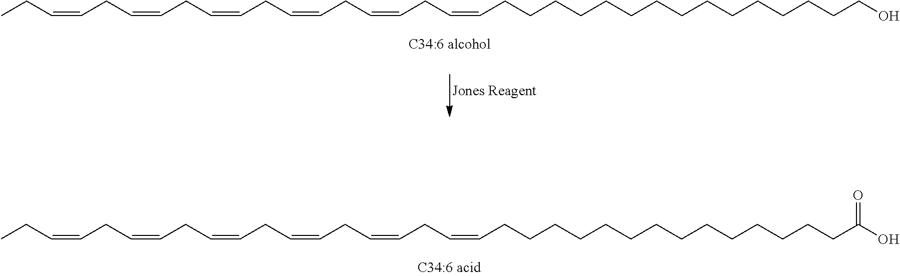 [SMILES: CC/C=C\C/C=C\C/C=C\C/C=C\C/C=C\C/C=C\CCCCCCCCCCCCCCCO ; CC/C=C\C/C=C\C/C=C\C/C=C\C/C=C\C/C=C\CCCCCCCCCCCCCCC(=O)O]

Preparation of Jones Reagent:

CrO₃ (10 g) was dissolved in concentrated H₂SO₄ (10 mL) and cooled to 0° C. (ice-water bath). Distilled water (30 mL) was added dropwise via addition funnel.

Procedure for Oxidation Using Jones Reagent:

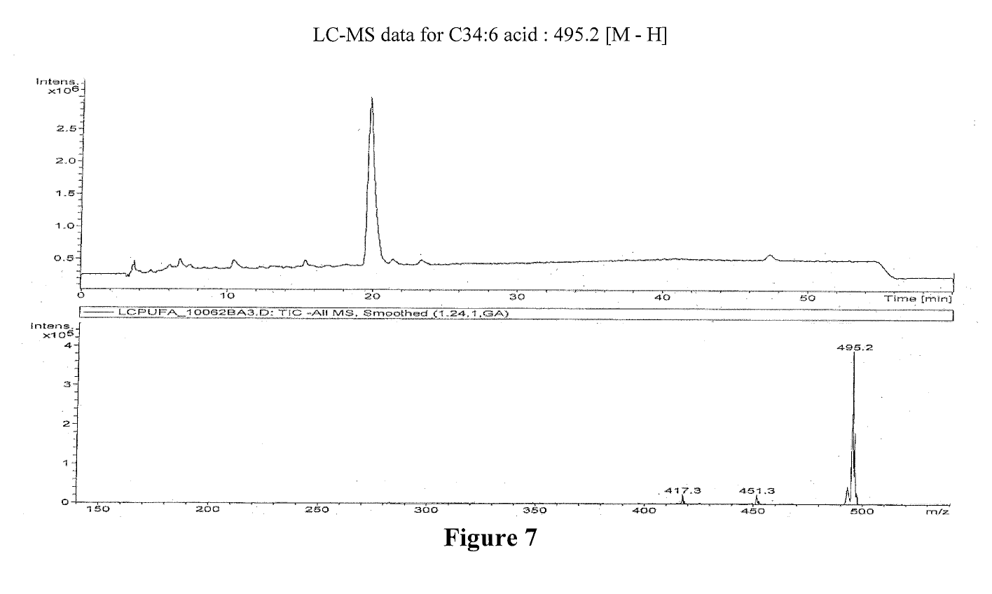

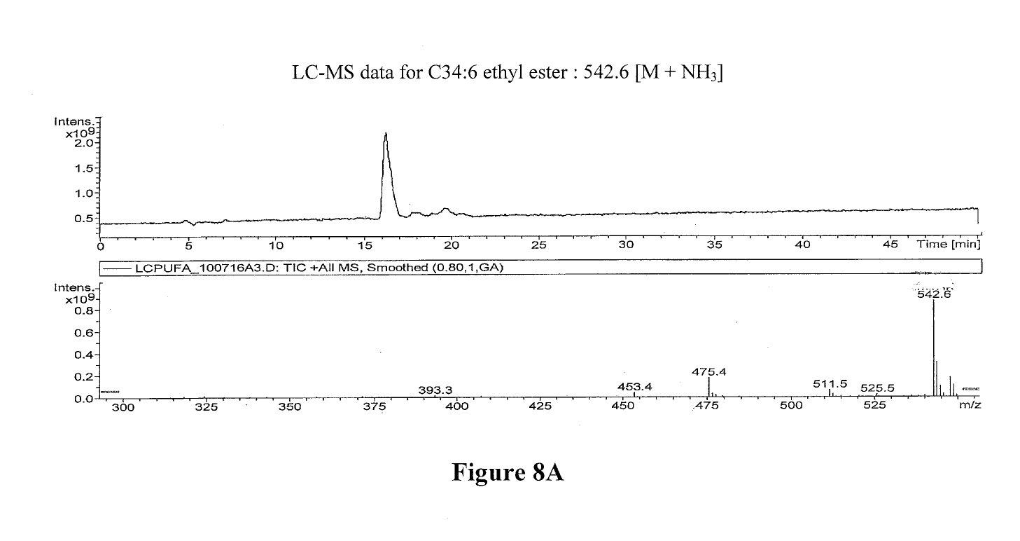

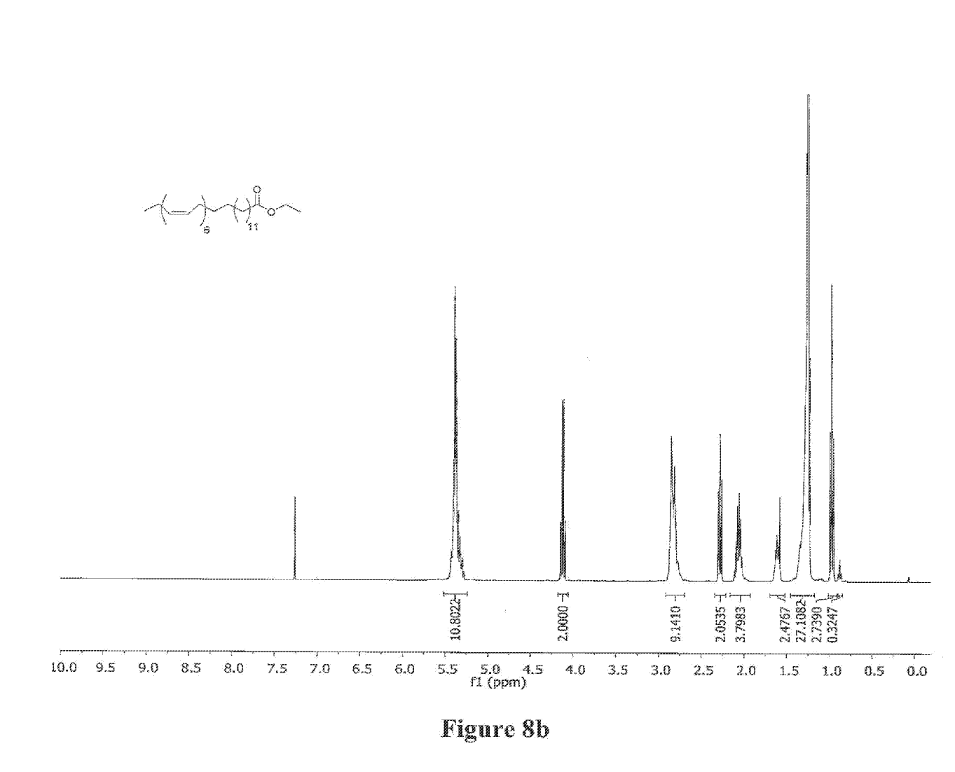

An oven dried 3-necked 100 mL round bottomed flask attached with a thermometer and an addition funnel was charged with C34:6 alcohol (2.8 g, 5.8 mmol). Acetone (25 mL) was added via a syringe. The mixture was cooled to 0° C. (ice-water bath). To this cold reaction mixture was added dropwise Jones reagent (8 mL). Common salt was added to the ice-water bath to maintain the temperature of the reaction mixture below 5° C. during the addition. The reaction mixture was allowed to stir for 2 h. The reaction was quenched by addition of water. Acetone was removed from the reaction mixture by rotary evaporator. The aqueous layer was extracted with ethyl acetate (70 mL×6). The combined organic layer was then dried (Na₂SO₄), filtered and concentrated under reduced pressure to obtain the product. The LC-MS data for C34:6 acid is shown in FIG. 7.

Esterification of C34:6 Acid:

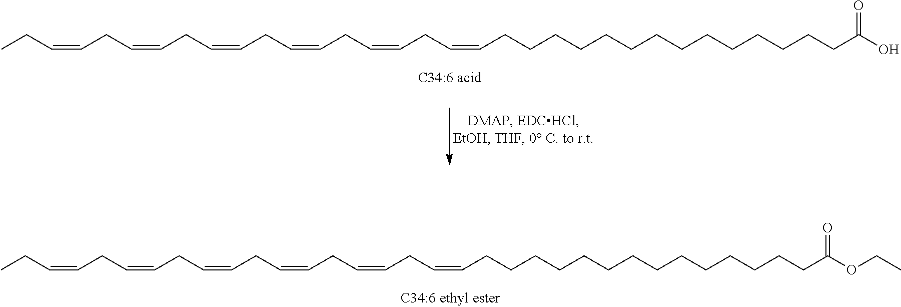 [SMILES: CC/C=C\C/C=C\C/C=C\C/C=C\C/C=C\C/C=C\CCCCCCCCCCCCCCC(=O)O ; CC/C=C\C/C=C\C/C=C\C/C=C\C/C=C\C/C=C\CCCCCCCCCCCCCCC(=O)OCC]

An oven dried 3-necked 100 mL round bottomed flask attached with a thermometer was charged with C34:6 acid (3.0 g, 6.05 mmol) Anhydrous THF (30 mL) was added and the mixture was allowed to cool to ° C. (ice-water bath). N,N-dimethylamino pyridine (0.81 g, 6.7 mmol) was added in one portion. After 10 min absolute ethanol (3.5 mL, 60.5 mmol) was added via a syringe. After 10 min EDC.HCl (1.7 g, 9.1 mmol) was added in one portion and the mixture was allowed to stir at 0 to 5° C. for 45 min. The cold bath was removed and the mixture was allowed to warm to room temperature and stir at that temperature for 4 h. The reaction was then quenched by addition of water and THF was removed by rotary evaporator. The aqueous layer was extracted with ethyl acetate (100 mL×6). The combined organic layer was then dried (Na₂SO₄), filtered and concentrated under reduced pressure to obtain the crude product. This crude product was purified by flash column chromatography using a gradient of hexanes to 3% ethyl acetate in hexanes to obtain 1.8 g of the product (56% yield).

¹H NMR (CDCl₃, 400 MHz, δ): 5.43-5.32 (m, 12H), 4.12 (q, J=6.8 Hz, 2H), 2.86-2.80 (m, 10H), 2.28 (t, J=7.6 Hz, 3H), 2.09-2.04 (m, 4H), 1.63-1.58 (m, 2H), 1.34-1.23 (m, 24H), 0.97 (t, 3H).

### Example 3

### Synthesis Procedures for EPA-Derived VLC PUFA

### Preparation of EPA Alcohol:

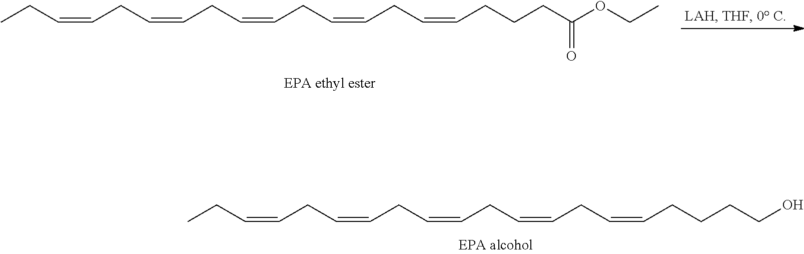

An oven dried 1 L round bottomed flask was charged with Lithium aluminum hydride (LAH) (42 g, 1100 mmol) in anhydrous THF (1 L). The flask was then cooled to 0° C. To this was added drop wise, a solution of EPA ethyl ester (332 g, 1000 mmol) in THF (1 L) via addition funnel. After the addition was complete the reaction was allowed to stir for X h at 0° C. The reaction was monitored by TLC. After the reaction was complete, it was quenched at 0° C. by slow drop wise addition of saturated aqueous solution of sodium sulfate. The mixture was then allowed to stir for 10-15 min and then filtered through Buchner funnel. The residue was washed with THF. The filtrate and washings were combined and concentrated under reduced pressure to obtain 291 g of the crude product (100% yield).

### Preparation of EPA Bromide:

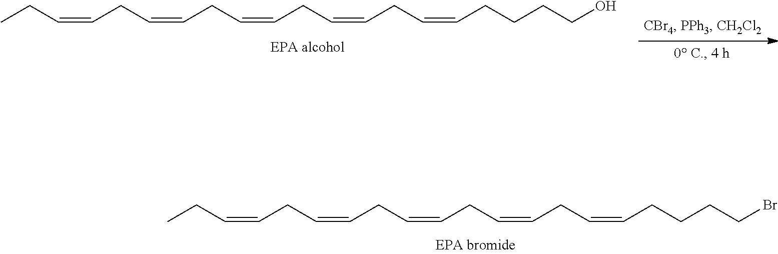 [SMILES: CC/C=C\C/C=C\C/C=C\C/C=C\C/C=C\CCCCO ; CC/C=C\C/C=C\C/C=C\C/C=C\C/C=C\CCCCBr]

An oven dried three-neck round bottomed flask was charged with the crude EPA alcohol (144 g, 500 mmol) and carbon tetrabromide (183 g, 550 mmol) in anhydrous CH₂Cl₂ (1000 mL). The flask was cooled to 0° C. (ice-water bath). To this mixture was added triphenylphosphine (144 g, 550 mmol) in 4 portions with an interval of 15 min between in each portion. The resulting mixture was allowed to stir at 0° C. The reaction was monitored by TLC. After 4 h the reaction mixture was concentrated under reduced pressure. Hexane was added and the mixture was cooled and filtered to remove triphenylphosphine oxide. This process was repeated 2 more times to remove majority of the byproduct. The crude product was then purified by flash column chromatography using hexanes to obtain 157 g of the product (90% yield).

### Preparation of 2-((8-chlorooctyl)oxy)tetrahydro-2H-pyran

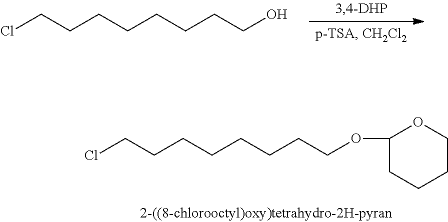 [SMILES: OCCCCCCCCCl ; ClCCCCCCCCOC1CCCCO1]

An oven dried 500 mL was charged with 8-chloro-1-octanol (48 g, 291 mmol). Anhydrous CH₂Cl₂ (400 mL) and the flask was cooled to 0° C. (ice-water bath). 3,4-dihydropyran (78 mL, 873 mmol) was added followed by p-toluenesulfonic acid monohydrate (5.6 g, 29.1 mmol). The reaction was monitored by TLC. After 4 h the reaction mixture was washed with saturated solution of NaHCO₃ (100 mL×3). The organic layer was again washed with H₂O (150 mL) and brine solution (150 mL). It was then dried (Na₂SO₄), filtered and concentrated under reduced pressure to obtain the crude product. This crude product was purified by flash column chromatography using a gradient of hexanes to 5% ethyl acetate in hexanes to obtain 65 g of the product (90% yield).

### Preparation of 8-tetrahydropyranyloxyoctyl magnesium chloride

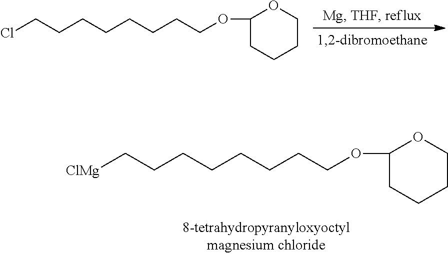

An oven dried 3 necked 500 mL round bottomed flask, attached with a thermometer, addition funnel and a water condenser was charged with magnesium turnings (10.9 g, 456 mmol). The flask was evacuated and filled with N₂. Anhydrous THF (172 mL) was added. The addition funnel was filled with a solution of 2-((8-chlorooctyl)oxy)tetrahydro-2H-pyran (28.4 g, 114 mmol) in anhydrous THF (56 mL). 20 mL of this solution was added dropwise to the reaction flask containing Mg turnings in THF at reflux. 1,2-dibromoethane (2.1 mL, 22.8 mmol) was added via a syringe. After 10 min the remaining solution from the addition funnel was added dropwise over a period of 30 min. The reaction mixture was allowed to reflux for 90 min to obtain 8-tetrahydropyranyloxyoctyll magnesium chloride.

### Coupling Reaction of EPA bromide with 8-tetrahydropyranyloxyoctyl magnesium chloride catalyzed by Li₂CuCl₄

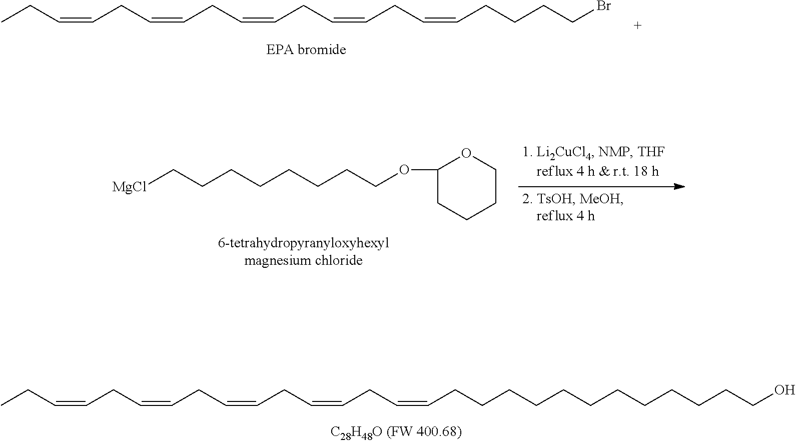

An oven dried 3 necked 1 L round bottomed flask, attached with a thermometer, addition funnel and a water condenser was charged with was charged with EPA bromide (20 g, 57 mmol). The flask was evacuated and filled with N₂ Anhydrous THF (80 mL) was added and the mixture was heated to 55 to 60° C. A solution of Li₂CuCl₄ (17 mL of 0.1 M solution in THF, 1.71 mmol) was added dropwise via a syringe. After 10 min N-methyl-2-pyrrolidone (22 mL, 228 mmol) was added dropwise via a syringe. This mixture was allowed to stir at 60° C. for 10 min and then was added a solution of 8-tetrahydropyranyloxyoctyl magnesium chloride (228 mL of 0.5 M solution in THF, 114 mmol) via addition funnel. The resulting reaction mixture was allowed to reflux for 4 h and then at r.t. for 16 h. The reaction was then quenched by addition of sat. solution of NH₄Cl (150 mL). Most of the THF from the reaction mixture was then removed by rotary evaporator. The aqueous layer was extracted with CH₂Cl₂ (150 mL×3). The combined organic phase was dried (Na₂SO₄), filtered and concentrated under reduced pressure to obtain the crude product. This crude THP protected C28:5 alcohol was then transferred to an oven dried 3 necked 500 mL round bottomed flask. Methanol (100 mL) was then added followed by p-toluenesulphonic acid monohydrate (6 g, 31.6 mmol). The mixture was allowed to reflux for 4 h. The reaction mixture was then concentrated under reduced pressure and CH₂Cl₂ (200 mL) was added. It was then washed with saturated solution of NaHCO₃ (150 mL×3) and brine (100 mL). The organic layer was dried (Na₂SO₄), filtered and concentrated under reduced pressure to obtain the crude product. It was purified by flash column chromatography using a gradient of hexanes to 10% ethyl acetate in hexanes to obtain 13 g of the desired product (57% yield).

### Bromination of C28:5 Alcohol:

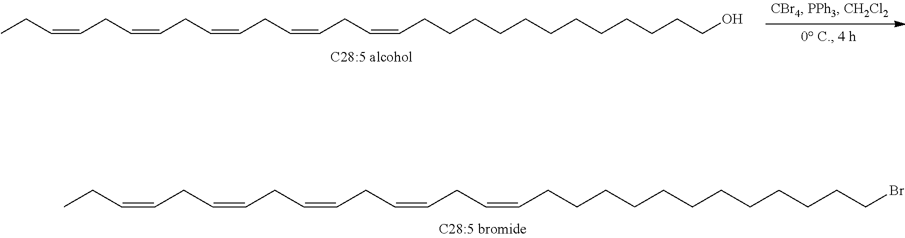 [SMILES: CC/C=C\C/C=C\C/C=C\C/C=C\C/C=C\CCCCCCCCCCCCO ; CC/C=C\C/C=C\C/C=C\C/C=C\C/C=C\CCCCCCCCCCCCBr]

An oven dried 3 necked 250 mL round bottomed flask attached with a thermometer was charged with C28:5 alcohol (13 g, 32.5 mmol) Anhydrous CH₂Cl₂ (40 mL) was added via a syringe and the mixture was cooled to 0° C. (ice-water bath). Carbon tetrabromide (13 g, 39 mmol) was added in one portion. To this mixture was added triphenylphosphine (10.2 g, 39 mmol) in 4 portions with the interval of 15 min between each portion. The reaction mixture was allowed to stir at 0 to 5° C. for 2 h. The reaction mixture was then concentrated under reduced pressure. Hexane (100 mL) was added and the mixture was filtered to remove triphenylphosphine oxide byproduct. This step was repeated to remove most of the byproduct. The crude product obtained was purified by flash column chromatography using hexanes to obtain 15 g of the desired product (100%).

### Coupling Reaction of C28:5 bromide with 6-tetrahydropyranyloxyhexyl magnesium chloride catalyzed by Li₂CuCl₄

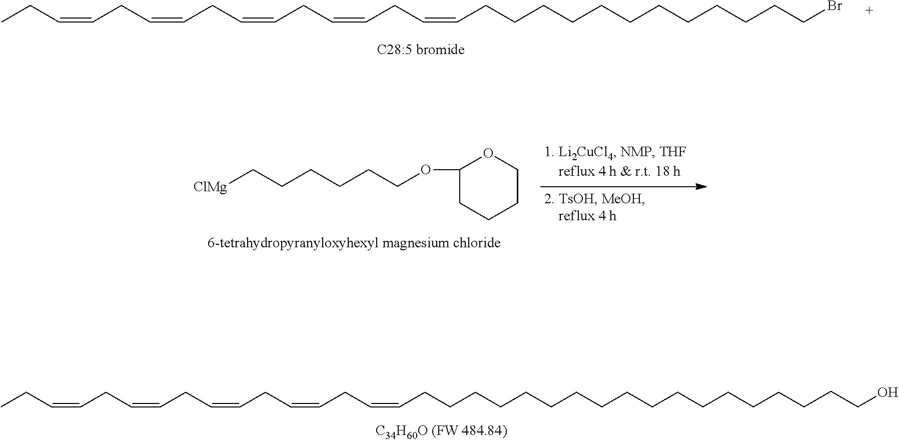

An oven dried 3 necked 1 L round bottomed flask, attached with a thermometer, addition funnel and a water condenser was charged with was charged with C28:5 bromide (15 g, 32.4 mmol). The flask was evacuated and filled with N₂ Anhydrous THF (50 mL) was added and the mixture was heated to 55 to 60° C. A solution of Li₂CuCl₄ (9.7 mL of 0.1 M solution in THF, 0.97 mmol) was added dropwise via a syringe. After 10 min N-methyl-2-pyrrolidone (12.5 mL, 130 mmol) was added dropwise via a syringe. This mixture was allowed to stir at 60° C. for 10 min and then was added a solution of 6-tetrahydropyranyloxyhexyl magnesium chloride (130 mL of 0.5 M solution in THF, 65 mmol) via addition funnel. The resulting reaction mixture was allowed to reflux for 4 h and then at r.t. for 18 h. The reaction was then quenched by addition of sat. solution of NH₄Cl (150 mL). Most of the THF from the reaction mixture was then removed by rotary evaporator. The aqueous layer was extracted with CH₂Cl₂ (150 mL×3). The combined organic phase was dried (Na₂SO₄), filtered and concentrated under reduced pressure to obtain the crude product. This crude THP protected C34:5 alcohol was then transferred to an oven dried 3 necked 500 mL round bottomed flask. Methanol (100 mL) was then added followed by p-toluenesulphonic acid monohydrate (6 g, 31.6 mmol). The mixture was allowed to reflux for 4 h. The reaction mixture was then concentrated under reduced pressure and CH₂Cl₂ (200 mL) was added. It was then washed with saturated solution of NaHCO₃ (150 mL×3) and brine (100 mL). The organic layer was dried (Na₂SO₄), filtered and concentrated under reduced pressure to obtain the crude product. It was purified by flash column chromatography using a gradient of hexanes to 10% ethyl acetate in hexanes to obtain 8.9 g of the desired product (57% yield).

### Oxidation of C34:5 Alcohol:

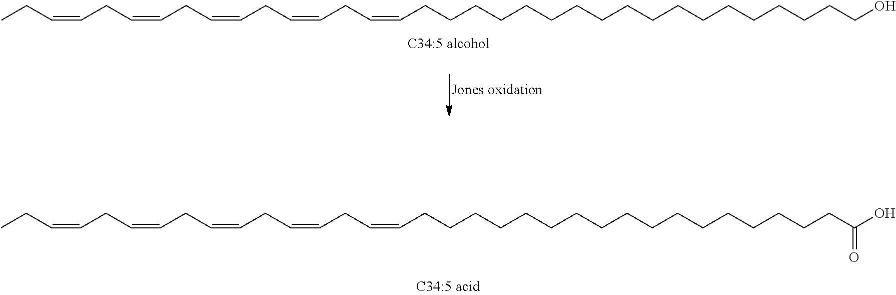 [SMILES: CC/C=C\C/C=C\C/C=C\C/C=C\C/C=C\CCCCCCCCCCCCCCCCCCO ; CC/C=C\C/C=C\C/C=C\C/C=C\C/C=C\CCCCCCCCCCCCCCCCCC(=O)O]

### Preparation of Jones Reagent:

CrO₃ (10 g) was dissolved in concentrated H₂SO₄ (10 mL) and cooled to 0° C. (ice-water bath). Distilled water (30 mL) was added dropwise via addition funnel.

### Procedure for Oxidation Using Jones Reagent:

An oven dried 3-necked 100 mL round bottomed flask attached with a thermometer and an addition funnel was charged with C34:5 alcohol (8.9 g, 18.4 mmol). Acetone (300 mL) was added via a syringe. The mixture was cooled to 0° C. (ice-water bath). To this cold reaction mixture was added dropwise Jones reagent (30 mL). Common salt was added to the ice-water bath to maintain the temperature of the reaction mixture below 5° C. during the addition. The reaction mixture was allowed to stir for 4 h. The reaction was quenched by addition of water. Acetone was removed from the reaction mixture by rotary evaporator. The aqueous layer was extracted with ethyl acetate (100 mL×6). The combined organic phase was then dried (Na₂SO₄), filtered and concentrated under reduced pressure to obtain the product.

¹H NMR (CDCl₃, 400 MHz, δ): 5.43-5.33 (m, 10H), 2.86-2.36 (m, 8H), 2.35 (t, J=7.6 Hz, 2H), 2.08-2.03 (m, 4H), 1.63 (quin, 3H), 1.31 (m, 28H), 0.97 (t, 3H).

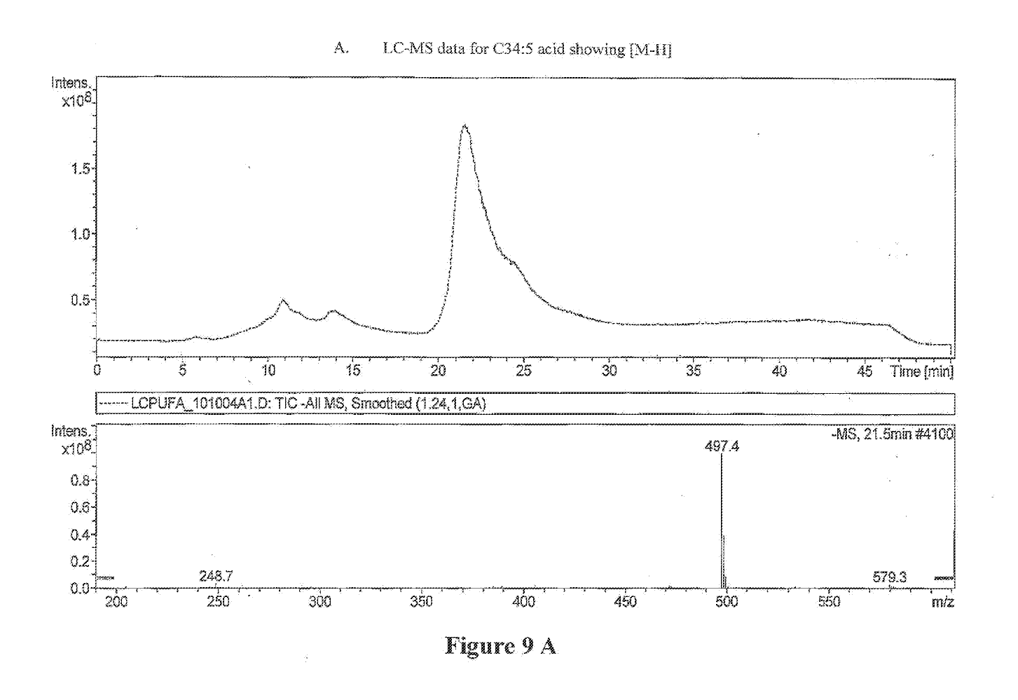

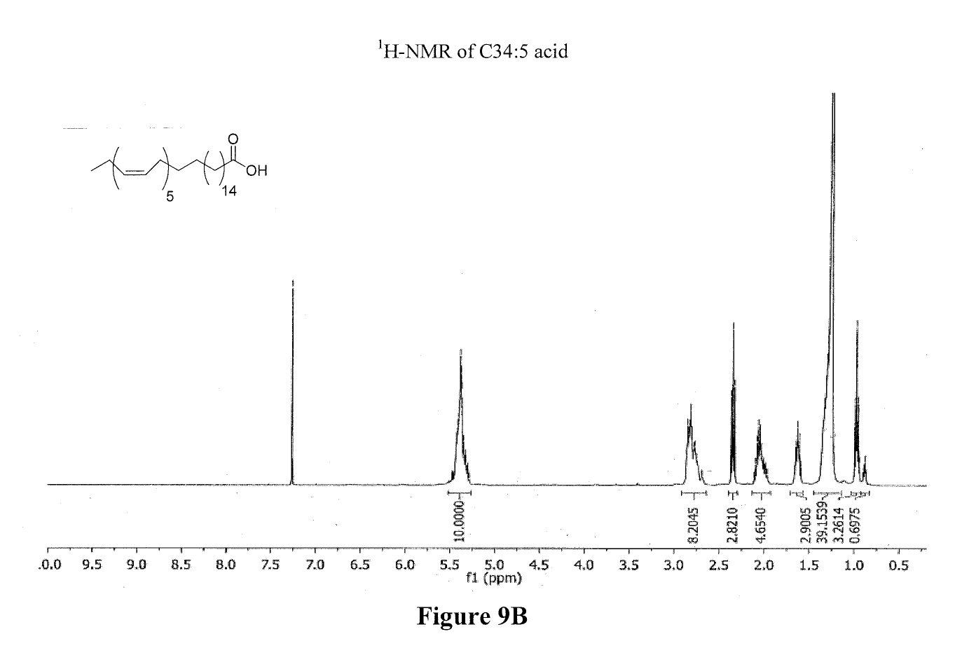

The NMR and LC-MS spectra for the C34:5 compound are shown in FIG. 9.

### Esterification of C34:5 Acid:

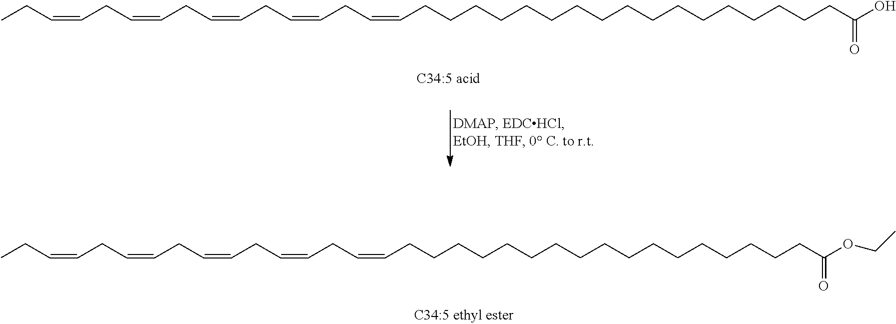 [SMILES: CC/C=C\C/C=C\C/C=C\C/C=C\C/C=C\CCCCCCCCCCCCCCCCCC(=O)O ; CC/C=C\C/C=C\C/C=C\C/C=C\C/C=C\CCCCCCCCCCCCCCCCCC(=O)OCC]

An oven dried 3-necked 100 mL round bottomed flask attached with a thermometer was charged with C34:5 acid (3.2 g, 6.4 mmol) Anhydrous THF (50 mL) was added and the mixture was allowed to cool to ° C. (ice-water bath). N,N-dimethylamino pyridine (0.86 g, 7.0 mmol) was added in one portion. After 10 min absolute ethanol (3.7 mL, 63.5 mmol) was added via a syringe. After 10 min EDC.HCl (1.83 g, 9.53 mmol) was added in one portion and the mixture was allowed to stir at 0 to 5° C. for 45 min. The cold bath was removed and the mixture was allowed to warm to r.t. and stir at that temperature for 4 h. The reaction was then quenched by addition of water and THF was removed by rotary evaporator. The aqueous layer was extracted with ethyl acetate (100 mL×6). The combined organic phase was then dried (Na₂SO₄), filtered and concentrated under reduced pressure to obtain the crude product. This crude product was purified by flash column chromatography using a gradient of hexanes to 3% ethyl acetate in hexanes to obtain 1.8 g of the product (53%).

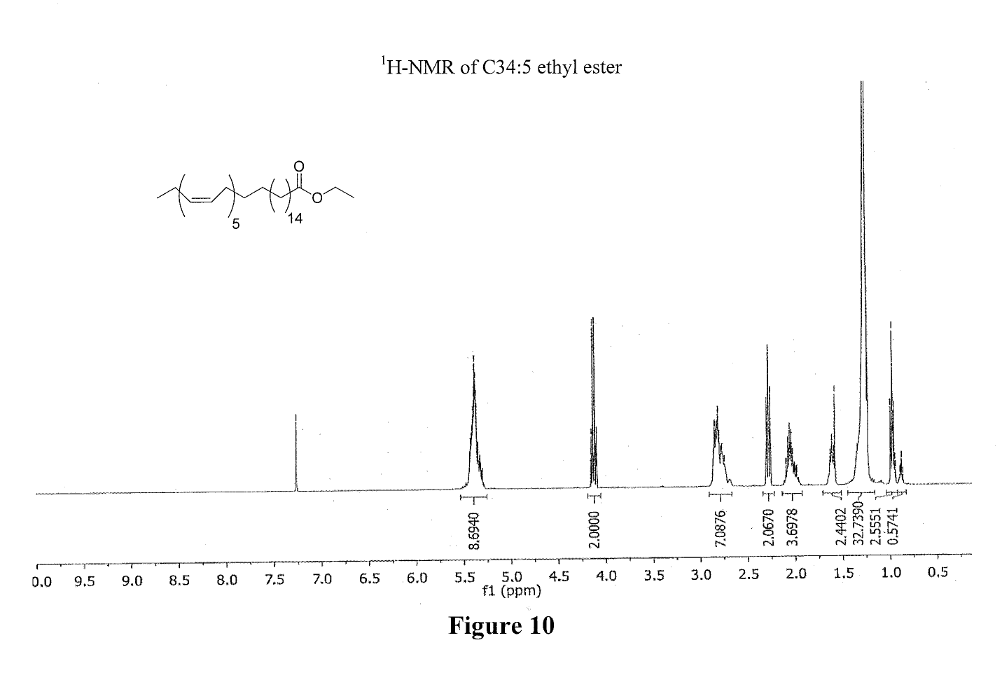

¹H NMR (CDCl₃, 400 MHz, δ): 5.42-5.34 (m, 10H), 4.12 (q, J=7.2 Hz, 14.4 Hz, 2H), 2.86-2.77 (m, 8H), 2.28 (t, J=7.6 Hz, 2H), 2.08-2.03 (m, 4H), 1.61 (t, J=7.6 Hz, 3H), 1.34-1.23 (m, 32H), 0.97 (t, 3H). FIG. 10 shows the NMR spectrum for the C34:5 ethyl ester.

### Example 4

### VLC-PUFA Tissue Accumulation

The accumulation of VLC-PUFAs in different tissues was assessed in animals fed ethyl esters of VLC-PUFAs. Ten Lewis male rats, 7-8 weeks old, were treated for ten days per os with C34:5 or C34:6 ethyl ester (see protocols MPK-MR1, for details). Blood samples were collected daily at 8 hrs after treatment.

Each animal tissue tested (Brain, Heart, Liver, Testis, Eyes) was dissected for further analysis after euthanasia. The tissues were frozen and weighed and then homogenized. The organic phase was extracted with methanol/chloroform, dried by evaporation, resuspended in acetonitrile/HCl and hexane. The lipophilic hexane layer was recovered, dried by evaporation and resuspended in acetone. The acetone solution, containing the biological extract, was derivatized with 3-Carbinol Pyridinium and 3 Bromo Pyridinium (see Yang et. al Anal. Chem. 79:5150-5157, 2007). The samples were derivatized with N-Methyl, 3-Carbinol Pyridinium salts, which result in the formation of N-Methyl, 3 pyridinium esters of the fatty acids. The introduction of a “fixed charged” (i.e., a Pyridinium ion) in the analytical moiety under investigation substantially improves the limits of detection for the LC-MS analysis

To perform a semi-quantitive determination of the analytes under evaluation and to improve the selectivity of the analysis, the samples were added with a deuterated reference standard, obtained via derivatization of standard reference VLC-PUFA 34:5 and 34:6 with N-CD3-3-carbinol pyridinium. The standards, 34:5 and 34:6 PUFAs, were desolved in acetone (0.01 mg/ml) and derivatized using the deuterated reagents (D3-Carbinol Pyridinium and D3-Br Pyridinium).

Both of the derivatives solutions were dried and resuspended in the mobile phase standard solution B. Each sample was analyzed in duplicate by HPLC-MS (Agilent MSD, equipped with an Electrospray Ionization source, and a single quadrupole analyzer). Experimental conditions for the HPLC-MS analysis were: Column: Agilent XBD, 150×2.1 mm, 5 micM particle size, Mobile phase: (A) HCOONH4 50 mM/acetonitrile, 1-1. (B) Acetonitrile, both solution contain 0.2% Formic acid, Flow rate: 0.4 ml/min, 25 C, Gradient: t0 85% B, t15 min 100% B, t18 min 100% B, t20 min 85% B, t25 min 85% B. Data were acquired in SIM mode monitoring m/z 602/605 (34:6 and 34:6 deuterated standard) and 604/607 (34:5 and correspondent duterated standard). Retention times were: 7.5 min for 34:6 and 10.3 min for 34:5.

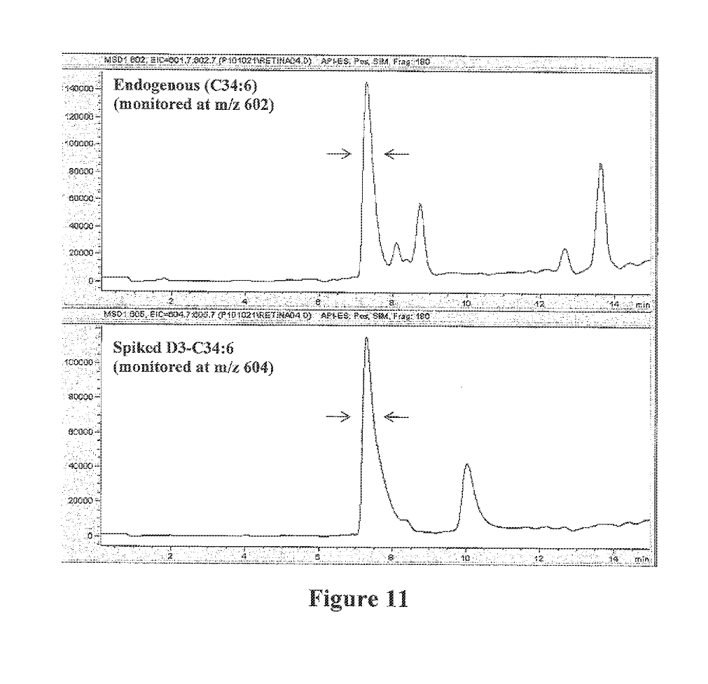

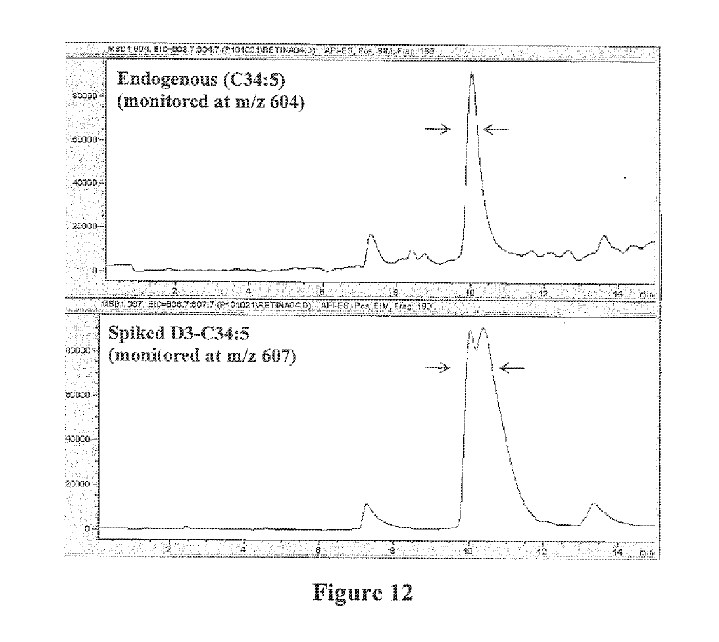

To obtain the analytical results, the VLC-PUFAs 34:5 and 34:6 and their corresponding deuterated standards were measured within the same 15 minutes chromatographic runs. The retention time for 34:6 (detected at m/z 602, standard deuterated at m/z 605) is 7.6 min, while the 34:5 (detected at m/z 604, standard deuterated at m/z 607) is 10.3 minutes. The semi-quantitative assessment of the VLC-PUFA is calculated from the ratio between the areas of the peaks of the 34:6 and its correspondent deuterated standard (D3-34:6) and from the area of 34:5 and its corresponding deuterated standard (D3-34:5). FIG. 11 shows the HPLC-MS analysis of 34:6 and D3-34:5 monitored selectively in channels 602 and 605, and FIG. 12 shows the HPLC-MS analysis of 34:5 and D3-34:5 monitored selectively in channels 604 and 607.

The amount of deuterated standard spiked in each sample is defined (in this case 250 ng were spiked in each sample) allowing a semi-quantitative assessment of the endogenous levels of VLC PUFA present in the sample.

A. Liver

FIG. 13 provides a visual comparison of the amount of 34:6 and 34:5 present in homogenates from liver for each animal in the three experimental groups. Basal levels (untreated animals) are close to the detection limit. However, the accumulation of the VLC-PUFA after 10 days treatment is evident.

B. Testis

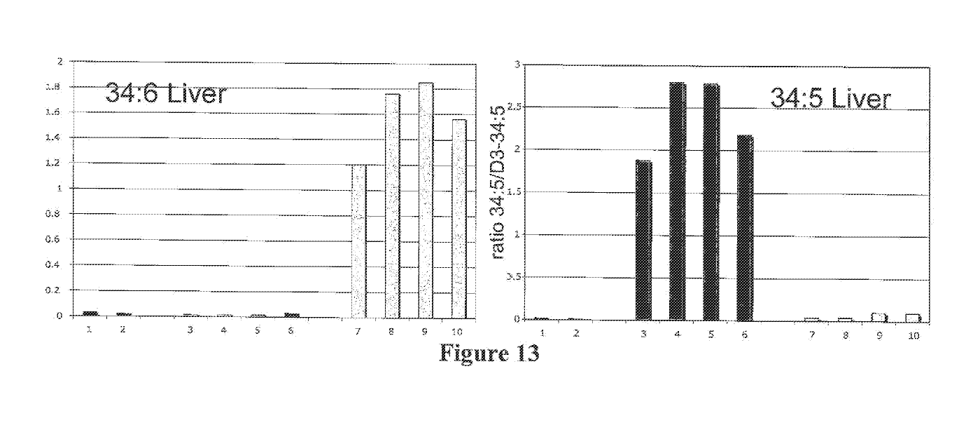

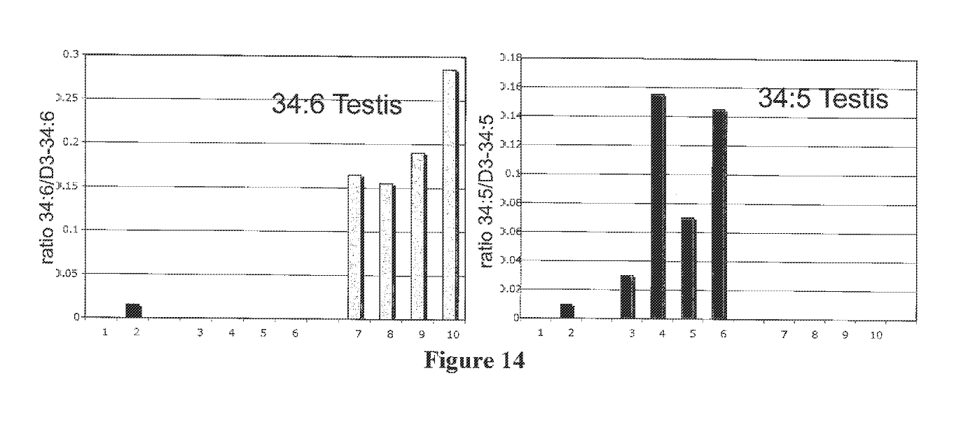

FIG. 14 provides a visual comparison of the amount of 34:6 and 34:5 present in homogenates from testis for each animal in the three experimental groups. Basal levels (untreated animals) are close to, or below the detection limit. As seen for the liver homogenates samples, the accumulation of the VLC-PUFA after 10 days treatment is evident.

C. Eye

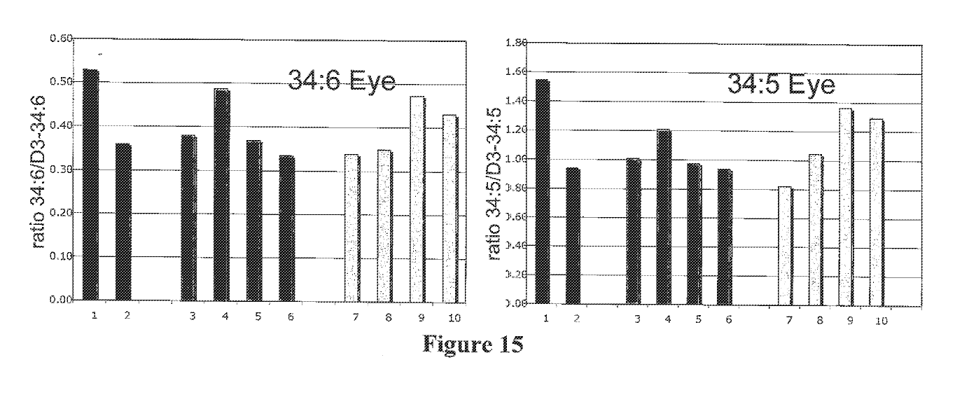

FIG. 15 provides a visual comparison of the amount of 34:6 and 34:5 present in homogenates from eyes from each animal in the three experimental groups. While the levels of these two VLC-PUFAs in the eye are well above the detection limit, accumulation after treatment is not apparent in this tissue.

D. Heart

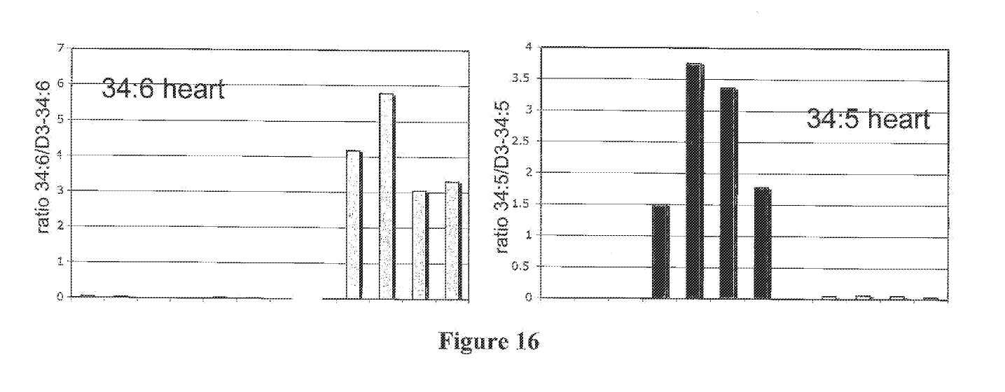

FIG. 16 provides a comparison of the amount of 34:6 and 34:5 present in homogenates from heart for each animal in the three experimental groups. Basal levels (untreated animals) are close or below detection limit. As seen for homogenate samples obtained from liver and testis, the accumulation of the administered VLC-PUFA after 10 days treatment is evident.

E. Plasma

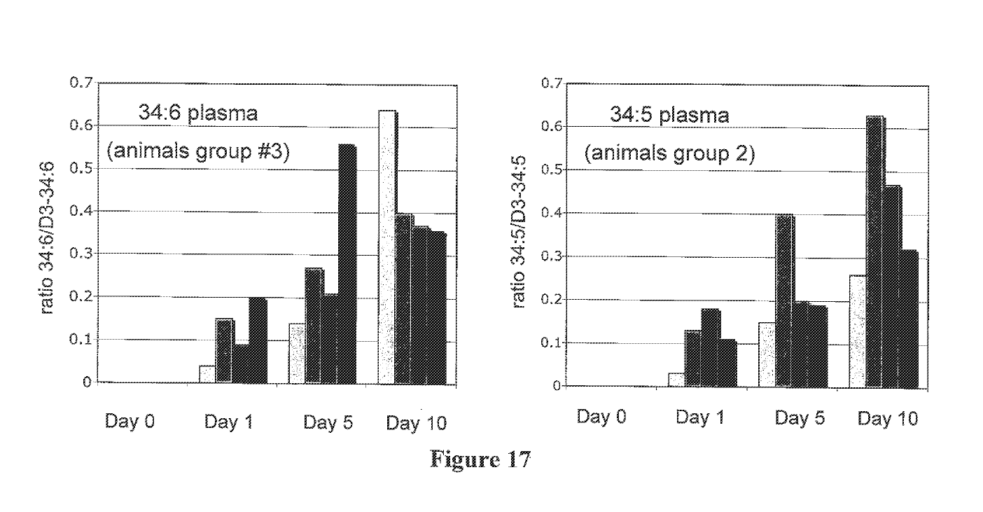

FIG. 17 shows the accumulation of the 34:6 and 34:5 VLC-PUFAs present in the plasma of treated animals. Accumulation of the VLC-PUFAs is evident only in the treated animals. (Group #2 received the 34:5 ester while Group#3 received the 34:6 ester)

These results demonstrate that following administration, the VLC-PUFAs are absorbed and circulate in the plasma of the animals. Taken together, the heart, liver and testis results indicate that the VLC-PUFAs administered to the animals are taken up into these organs, and the administered VLC-PUFAs are evident in these tissues.

### Example 5

### VLC-PUFA In Vitro Neuroprotection

Protection from various neurological injuries by the pretreatment of subjects with VLC-PUFAs was evaluated in timed-pregnant female Long Evans rats obtained from Charles River Laboratories (Wilmington, Mass.), housed individually at the Boulder BioPath animal research facility, and maintained on a 12 h:12 h light schedule with ad libitum access to rat chow and water. All animal protocols were approved by the Animal Care and Use Committee at Boulder BioPath.

Primary Hippocampal Neuronal Culture:

Primary hippocampal neurons were harvested from embryonic day 17-18 rat fetuses using a modified version of previously described protocols (Banker and Cowan, 1977; Brewer et al., 1993) that preferentially select for neuronal growth over glial growth (Brewer, 1997). Pregnant dams were anesthetized via isoflurane on gestational day 18, and the rat fetuses were harvested by Cesarean section. Brains were removed and placed in ice-cold (4° C.) Hibernate EB (Brain Bits, Inc., Springfield, Il, USA) until all of the hippocampi were dissected. During the dissection, brain tissue was kept on an ice-cold block in Hibernate E solution. Dissected hippocampal tissue was washed once in room-temperature Hibernate E (without calcium, Brain Bits, Inc.) to remove divalent cations, and then incubated at 37° C. in a solution consisting of 0.17% trypsin in Hybernate E (without calcium) for 10 minutes. The tissue was subsequently washed once with ice-cold Hybernate E (without calcium) containing 15% fetal bovine serum (Atlas Biologicals, Fort Collins, Colo., USA) and then dissociated by trituration in the same media. Dispersed cell suspensions were centrifuged for 2 minutes at 200 g at 4° C., and resuspended in ice-cold Neurobasal medium (Invitrogen, Carlsbad, Calif., USA) supplemented with 1×B27 (Invitrogen), 100 μg/ml penicillin-streptomycin (Invitrogen), 0.5 mM L-glutamine (Invitrogen) and 0.025 mM L-glutamate (Sigma, St. Louis, Mo., USA). Viable cell number and concentration were determined by trypan blue exclusion and counted using an automated cell counter (Countess, Invitrogen). Cell suspensions were diluted in warm Neurobasal/B27 media and plated at a density of 30,000 cells per well in 96-well plates (Falcon Optilux plates, BD Biosciences, Bedford, Mass., USA) coated with laminin (Sigma) and poly-D-lysine (Sigma). Cultures were maintained in a temperature- (37° C.), humidity- (−100%), and gas- (5% CO₂, 9% O₂) controlled incubator. Half of the media was removed and replaced with fresh media every other day. All cultures were maintained for 3 days in vitro (DIV) in glutamate-containing media and then maintained an additional 7 DIV (glutamate neurotoxicity experiments) or 8 DIV (hypoxia) in Neurobasal/B27 media without glutamate.

Treatments:

Very long-chain polyunsaturated fatty acids (VLCPUFAs; 34:6n3, 34:5n3, 28:6n3, 28:5n3, and 28:4n6) were obtained as the free fatty acid and were stored at −20° C. until use. Each VLC-PUFA was dissolved in methanol to an initial concentration of 100 mM and then diluted serially in methanol to obtain 1000× stocks for each concentration tested. These were diluted further in media to obtain the appropriate concentration within the culture wells (final concentrations of 30 μM, 10 μM, 3 μM, 1 μM, 0.3 μM, and 0.1 μM). Controls received methanol similarly diluted in media (final concentration 0.1%). The VLC-PUFAs were added to the cultures 48 hours prior to glutamate insult or hypoxia exposure (described below), and a 50% media change occurred immediately prior to hypoxia exposure or immediately following glutamate exposure.

Glutamate Neurotoxicity:

Cultures were treated with L-glutamic acid (50 μM, pH 7.0) for 60 minutes followed by a complete media change containing the appropriate concentrations of VLC-PUFAs. Cells were assayed for viability and mitochondrial health 24 hours after the glutamate treatment.

Hypoxia:

Cultures were placed in a hypoxic chamber (C-Chamber, BioSpherix, Inc., Redfield, Il, USA) that was set to control the oxygen level to ≦0.3% and the carbon dioxide level to 5.0%. Cultures remained in the hypoxic chamber for 48 hours until assayed for cell viability and mitochondrial health.

Cell Viability and Mitochondrial Health Assay:

Following glutamate exposure or hypoxia, cultures were aspirated with a 96-well plate washer (BioTek, Winooski, Vt., USA) and incubated for 15 minutes at 37° C. with phenol-free Neurobasal media (Invitrogen) containing one of two dye mixtures: 1) Hoechst dye (1:2000 of a 10 μg/ml solution, Invitrogen) and propidium iodide (PI; 1:500 of a 1 μg/ml solution; Invitrogen), or 2) Hoechst dye and tetramethylrhodamine ethyl ester (TMRE; 100 nM; Invitrogen). These dyes identify total cells (Hoechst), dying or dead cells (PI), and functional mitochondria (TMRE). After 15 minutes, plates were aspirated and washed once (Hoechst and PI) or twice (Hoechst and TMRE) with warm Hank's balanced salt solution (HBSS) by a 96-well plate washer (final addition of 50 μL HBSS), and scanned on a fluorimeter (Fluoroskan Ascent, ThermoFisher, Waltham, Mass., USA) using the appropriate excitation/emission filter pair for each fluorescent dye.

Statistical Analysis:

Raw data were normalized to the injured vehicle control group (0 μM VLC-PUFA) within each experiment and expressed graphically as mean percentages ±the standard error of the mean (SEM). Statistical analysis was completed using SPSS 16.0 software (IBM, Armonk, N.Y., USA), and graphs were assembled with GraphPad Prism 5.4 (GraphPad Inc., La Jolla, Calif., USA). Significant differences from controls were determined by one-way analysis of variance (ANOVA) followed by Tukey's post hoc tests, and α levels (p) less than 0.05 were considered significant. Some of the data sets did not exhibit normal distribution frequencies. Therefore, these data sets were transformed with log₁₀ or square-root calculations to improve their frequency distributions prior to analysis.

### A Inhibition of Cell Death Following Glutamate Exposure

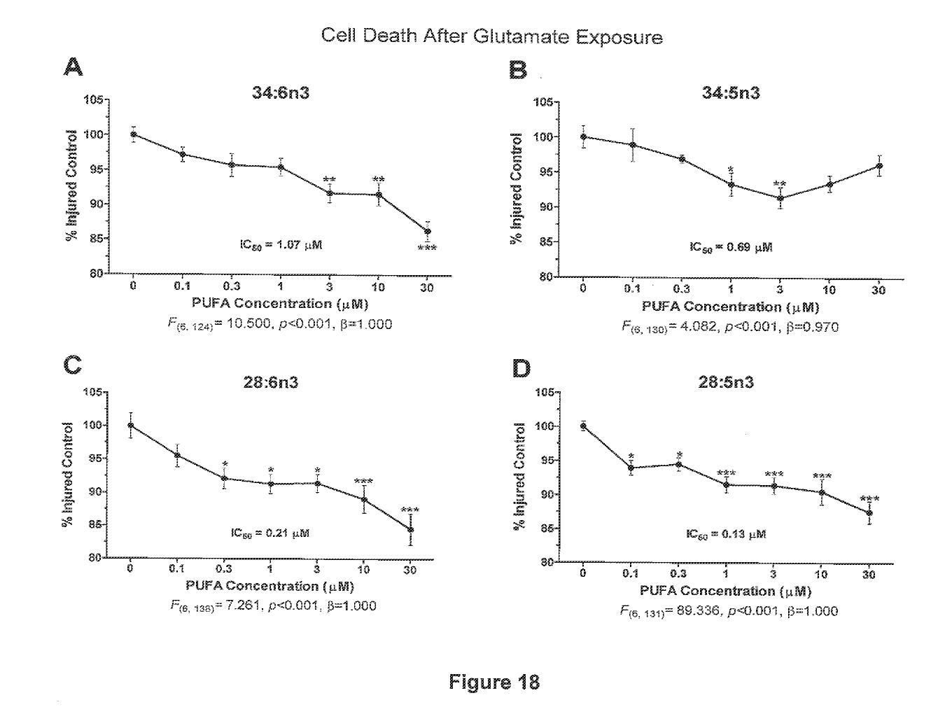

Very long-chain PUFA pretreatment inhibited cell death due to glutamate exposure in primary hippocampal cell cultures. FIG. 18A shows that the VLC-PUFA 34:6n3 significantly inhibited cell death at concentrations ≧3.0 μM and exhibited a half-maximal inhibitory concentration (IC50) of 1.07 μM. FIG. 18B shows that the VLC-PUFA 34:5n3 significantly inhibited cell death at concentrations of 1.0 μM and 3.0 μM and exhibited an IC50 of 0.69 μM. FIG. 18C shows that the VLC-PUFA 28:6n3 significantly inhibited cell death at concentrations ≧0.3 μM and exhibited an IC50 of 0.21 μM. FIG. 18D shows that the VLC-PUFA 28:5n3 significantly inhibited cell death at concentrations ≧0.1 μM and exhibited an IC50 of 0.13 μM. Referring to FIG. 18, one-way ANOVA parameters are illustrated below each graph, and the results of Tukey's post hoc pair-wise comparisons to controls are indicated by stars (*, p<0.05; **, p<0.01; ***, p<0.001). Error bars represent the SEM. N=16-20 over 2 separate culture runs.

### B. VLC-PUFA Promotes Healthy Mitochondrial Function

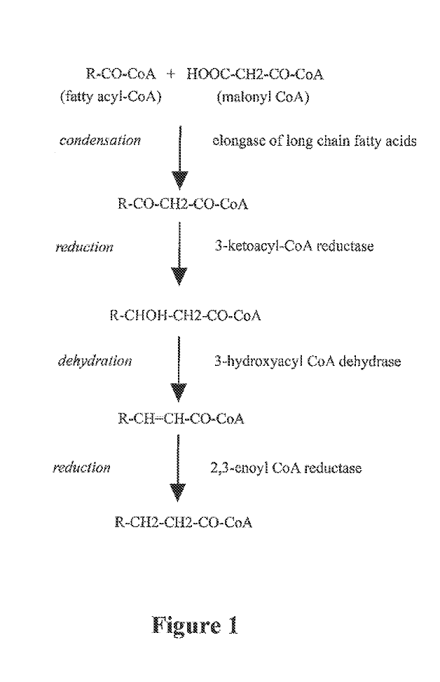

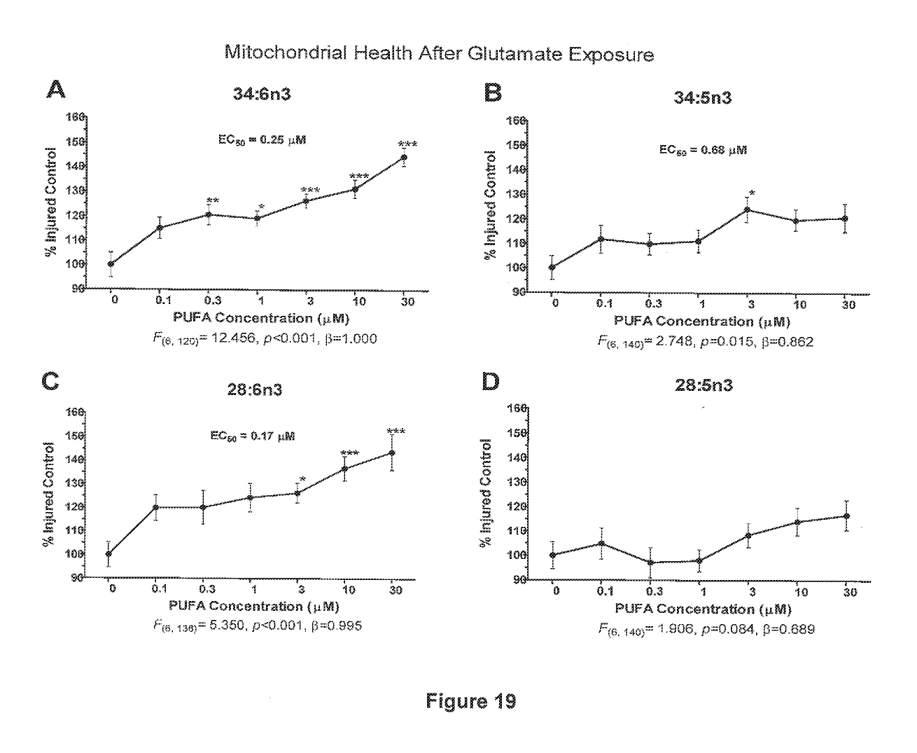

Very long-chain PUFA pretreatment promoted healthy mitochondrial function when measured after glutamate-induced neurotoxicity in primary hippocampal cell culture. FIG. 1pA shows that the VLC-PUFA 34:6n3 significantly increased mitochondrial health at concentrations ≧0.3 μM and exhibited a half-maximal effective concentration (EC50) of 0.25 μM. FIG. 19B shows that the VLC-PUFA 34:5n3 significantly increased mitochondrial health at a concentration of 3.0 μM and exhibited an EC50 of 0.68 μM. FIG. 19C shows that the VLC-PUFA 28:6n3 significantly increased mitochondrial health at concentrations ≧3.0 μM and exhibited an EC50 of 0.17 μM. FIG. 19D shows that the VLC-PUFA 28:5n3 did not significantly alter mitochondrial health after glutamate insult. Referring to FIG. 19, one-way ANOVA parameters are illustrated below each graph, and the results of Tukey's post hoc pair-wise comparisons to controls are indicated by stars (*, p<0.05; **, p<0.01; ***, p<0.001). Error bars represent the SEM. N=16-20 over 2 separate culture runs.

### C. VLC-PUFAs Inhibit Hypoxic Cell Death

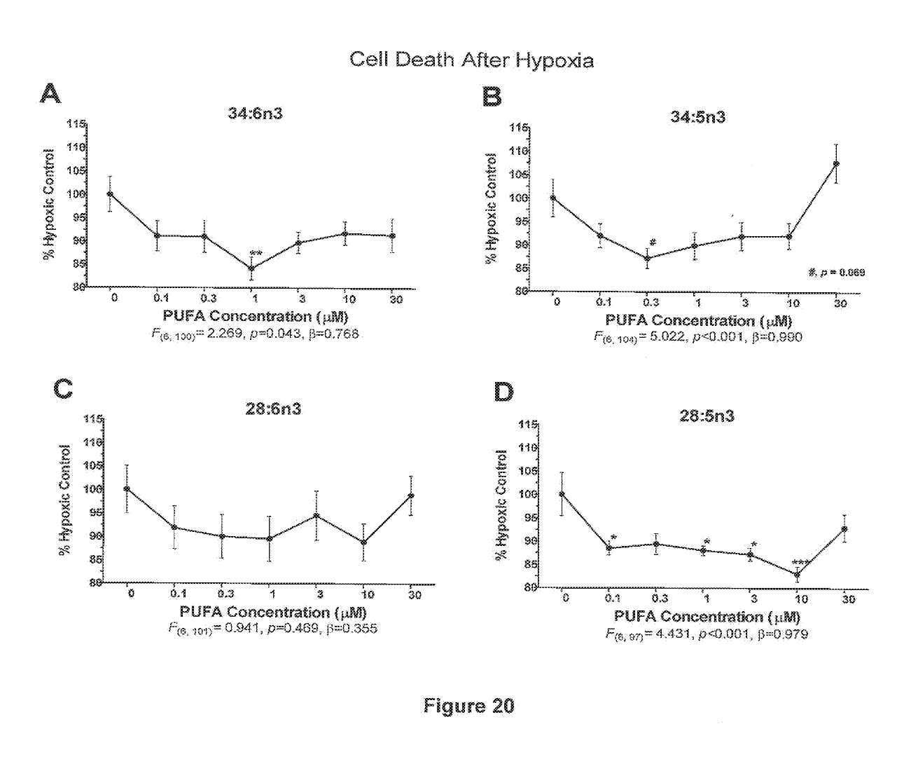

Very long-chain PUFA pretreatment generally inhibited cell death due to hypoxia in primary hippocampal cells, but at lower levels than those observed after glutamate exposure. FIG. 20A shows that the VLC-PUFA 34:6n3 significantly inhibited cell death at a concentration of 1.0 μM. FIG. 20B shows that the VLC-PUFA 34:5n3 exhibited a main effect of treatment, but only one concentration (0.3 μM) approached a significant difference when compared to controls. FIG. 20C shows that the VLC-PUFA 28:6n3 did not inhibit hypoxia-induced cell death at any concentration tested. FIG. 20D shows that the VLC-PUFA 28:5n3 significantly inhibited cell death at concentrations of 0.1 μM to 10 μM. Referring to FIG. 20, one-way ANOVA parameters are illustrated below each graph, and the results of Tukey's post hoc pair-wise comparisons to controls are indicated by stars (*, p<0.05; **, p<0.01; ***, p<0.001). Error bars represent the SEM. N=11-15 over 2 separate culture runs.

### D. VLC-PUFAs Promote Mitochondrial Health

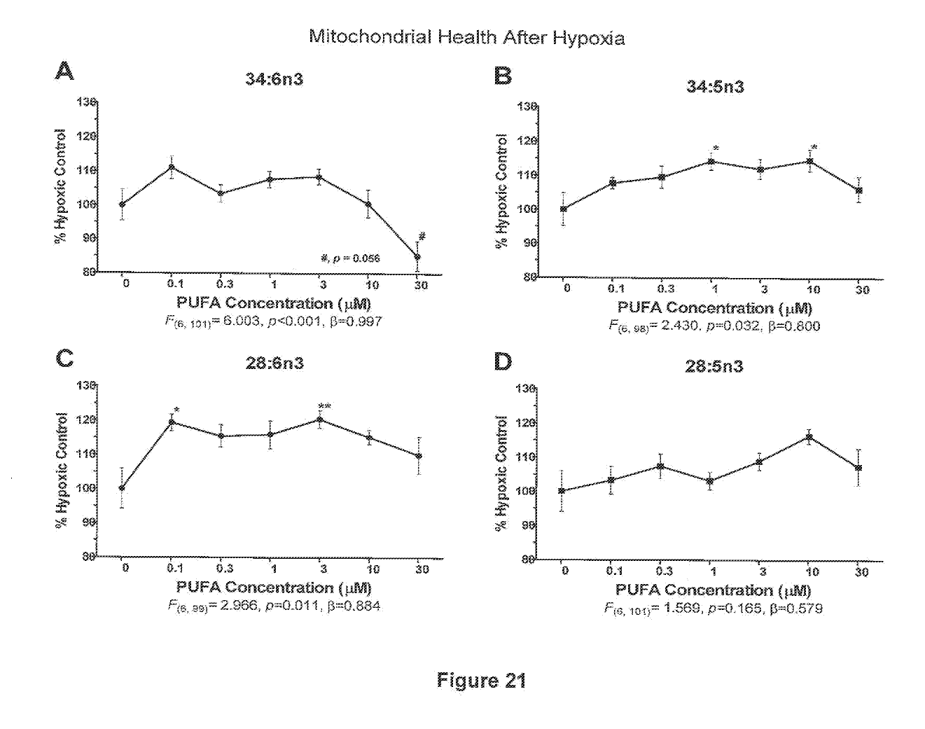

Very long-chain PUFA pretreatment generally promoted mitochondrial health after hypoxia in primary hippocampal cells, but at lower levels than those observed after glutamate exposure. FIG. 21A shows that the VLC-PUFA 34:6n3 did not significantly alter mitochondrial health at the concentrations tested. FIG. 21B shows that the VLC-PUFA 34:5n3 significantly increased mitochondrial health at concentrations of 1.0 μM and 10 μM. FIG. 21C shows that the VLC-PUFA 28:6n3 significantly increased mitochondrial health at concentrations of 0.1 μM and 3.0 μM. FIG. 21D shows that the VLC-PUFA 28:5n3 did not significantly alter mitochondrial health at the concentrations tested. Referring to FIG. 21, one-way ANOVA parameters are illustrated below each graph, and the results of Tukey's post hoc pair-wise comparisons to controls are indicated by stars (*, p<0.05; **, p<0.01). Error bars represent the SEM. N=11-15 over 2 separate culture runs.

### E. VLC-PUFAs Inhibit Neuronal Cell Death

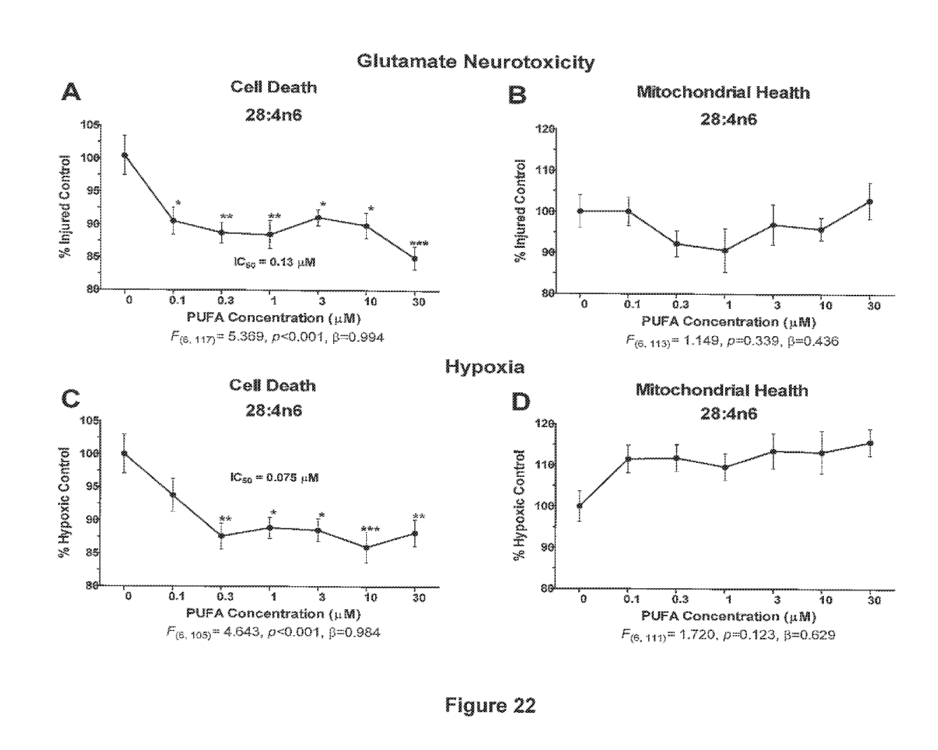

Very long-chain PUFA 28:4n6 inhibited cell death due to either glutamate exposure or hypoxia in primary hippocampal cell cultures, but did not alter mitochondrial health overall. FIG. 22A shows that the VLC-PUFA 28:4n6 significantly inhibited cell death due to glutamate exposure at concentrations ≧0.1 μM and exhibited a half-maximal inhibitory concentration (IC50) of 0.13 μM. FIG. 22B shows that the VLC-PUFA 28:4n6 did not significantly improve reductions in mitochondrial health due to glutamate exposure at the concentrations tested. FIG. 22C shows that the VLC-PUFA 28:4n6 significantly inhibited cell death due to hypoxia at concentrations ≧0.3 μM and exhibited an IC50 of 0.075 μM. FIG. 22D shows that the VLC-PUFA 28:4n6 did not significantly improve reductions in mitochondrial health due to hypoxia at the concentrations tested. Referring to FIG. 22, one-way ANOVA parameters are illustrated below each graph, and the results of Tukey's post hoc pair-wise comparisons to controls are indicated by stars (*, p<0.05; **, p<0.01; ***, p<0.001). Error bars represent the SEM. N=14-17 over 2 separate culture runs.

### REFERENCES

1. Yeh, M. C. P.; Knochel, P.; Santa, L. E. Tetrahedron Lett. 1988, 29(32), 3887-3890.
2. Quinton, P.; Le Gall, T. Tetrahedron Lett. 1991, 32(37), 4909-4912.
3. Knochel, P.; Rozema, M. J.; Tucker, C. E.; Retherford, C.; Furlong, M.; Achyutharao, S. Pure & Appl. Chem. 1992, 64(3), 361-369 and references therein.
4. Yasuda, M.; Onishi, Y.; Ueba, M.; Miyai, T.; Baba, A. J. Org. Chem. 2001, 66, 7741-7744.
5. Zhou, J.; Fu, G. J. Am. Chem. Soc. 2003, 125, 12527-12530.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method of coupling a long chain hydrocarbon to an extender hydrocarbon to form a very long chain hydrocarbon having at last 28 carbon atoms comprising:
i. reacting a long chain hydrocarbon, having a nucleophillic displacement group on one end, with an extending reagent,
ii. wherein the extending reagent comprises a nucleophillic attacking group and an extender hydrocarbon chain having a protecting functional group on one end, and
iii. wherein the coupling is done in the presence of an activating catalyst.

2. The method as recited in claim 1 wherein the protecting functional group is selected from an ester and an ether.

3. The method as recited in claim 1 where the nucleophillic displacement group is a halogen.

4. The method as recited in claim 1 wherein the extending reagent is selected from a Grignard extender reagent and a zinc extender reagent.

5-38. (canceled)

39. A method of lengthening a polyunsaturated fatty acid to form an ester comprising:
a. reducing an ester of a first polyunsaturated fatty acid to form a primary alcohol;
b. oxidizing the primary alcohol to form an aldehyde; and
c. contacting the aldehyde with an extender reagent to form an elongated ester.

40. The method of claim 39, further comprising transesterifying a glyceride to form the first polyunsaturated fatty acid.

41-43. (canceled)

44. The method of claim 40, wherein the glyceride is derived from an algae cultured in a fermentation medium.

45-61. (canceled)

62. The method of claim 39, wherein the reducing is conducted in the presence of at least one of lithium aluminum hydroxide (LAH) and tetrahydrofuran (THF).

63-64. (canceled)

65. The method of claim 39, wherein the oxidizing is conducted in the presence of at least one of dimethyl sulfoxide (DMSO), oxalyl chloride and triethylamine (Et₃N).

66-68. (canceled)

69. The method of claim 39, wherein the contacting is conducted in the presence of at least one of CuCN, LiC, titanium propoxide (Ti(i-OPr)₄) and tetrahydrofuran (THF).

70-75. (canceled)

76. The method of claim 39, further comprising hydrolyzing the elongated ester to form an elongated second polyunsaturated fatty acid.

77. The method of claim 76, further comprising removal of an alcohol moiety from the elongated ester in the presence of at least one of diphenylsilane hydrochloride (Ph₂Si HCl), indium trichloride (InCl₃), and dichloroethane (CH₂ClCH₂Cl).

78. An isolated C28-C38 PUFA, an analog thereof, a derivative thereof, or a salt thereof produced by the method of claim 39.

79. (canceled)

80. A composition comprising at least one isolated C28-C38 PUFA according to claim 78, an analog thereof, a derivative thereof, or a salt thereof, and optionally a compound selected from SDA, GLA, ARA, DPAn-6, DPAn-3, DTAn-6, DHA, and EPA.

81-84. (canceled)

85. An oil comprising at least about 10 μg, at least about 50 μg, or at least about 100 μg of at least one isolated VLC-PUFA according to claim 78 per gram of oil.

86. The oil of claim 85, wherein the isolated VLC-PUFA is selected from C28:4, 5 and 6 n3 and n6 PUFAs, C32:4, 5 and 6 n3 and n6 PUFAs, C34:4, 5 and 6 n3 and n6 PUFAs; and C36:4, 5 and 6 n3 and n6 PUFAs.

87-135. (canceled)

136. A method comprising clinical dietary management of retinal health in an individual in need thereof by administering an effective amount of a VLC-PUFA to said individual.

137. A method comprising clinical dietary management of degenerative eye condition selected from macular degeneration and Stargardt's disease in an individual in need thereof by administering an effective amount of a VLC-PUFA to said individual.

138-144. (canceled)

145. The method of claim 136, wherein the VLC-PUFA is selected from C34:6n3, C34:5n3, C34:4n6, C28:6n3, C28:5n3 and 28:4n6.

146-152. (canceled)

153. The method of claim 145, wherein the isolated VLC-PUFA is chemically synthesized de novo by a chemical synthesis of claim 1.
